(19) United States
(12) Patent Application Publication
(10) Pub. No.: US 2010/0113421 A1
(43) Pub. Date: May 6, 2010

(54) NON-NUCLEOSIDE REVERSE TRANSCRIPTASE INHIBITORS

(76) Inventors: Theresa M. Williams, Harleysville, PA (US); Xu-Fang Zhang, Dresher, PA (US); Vanessa E. Obligado, Oreland, PA (US); Rebecca A. Poehnelt, Mercerville, NJ (US)

Correspondence Address: MERCK, P O BOX 2000, RAHWAY, NJ, 07065-0907, US

(21) Appl. No.: 12/444,014

(22) PCT Filed: Oct. 2, 2007
(86) PCT No.: PCT/US07/21208

§ 371 Date: Apr. 2, 2009

Related U.S. Application Data
(60) Provisional application No. 60/849,902, filed on Oct. 6, 2006.

(51) Int. Cl. A61K 31/55; C07D 207/00; A61K 31/40; C07D 401/12; A61K 31/454; C07D 495/04

(52) U.S. Cl. 514/215; 548/537; 514/423; 546/208; 514/326; 540/593

(57) ABSTRACT

Compounds of Formula (I) are HIV reverse transcriptase inhibitors, wherein X, R¹, R², R³, R⁴ and R⁵ are defined herein. The compounds of Formula (I) and their pharmaceutically acceptable salts are useful in the inhibition of HIV reverse transcriptase, the prophylaxis and treatment of infection by HIV and in the prophylaxis, delay in the onset or progression, and treatment of AIDS. The compounds and their salts can be employed as ingredients in pharmaceutical compositions, optionally in combination with other antivirals, immunomodulators, antibiotics or vaccines.

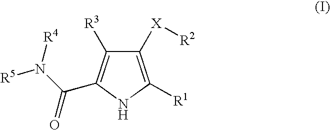

This application claims the benefit of U.S. Provisional Application No. 60/849,902, filed Oct. 6, 2006, the disclosure of which is hereby incorporated by reference in its entirety.

### FIELD OF THE INVENTION

The present invention is directed to certain pyrroles and their pharmaceutically acceptable salts and their use for the inhibition of HIV reverse transcriptase, the prophylaxis of HIV infection and HIV replication, the treatment of HIV infection and HIV replication, the prophylaxis of AIDS, the treatment of AIDS, and the delay in the onset and/or progression of AIDS.

### BACKGROUND OF THE INVENTION

The retrovirus designated human immunodeficiency virus (HIV), particularly the strains known as HIV type-1 (HIV-1) and type-2 (HIV-2) viruses, have been etiologically linked to the immunosuppressive disease known as acquired immunodeficiency syndrome (AIDS). HIV seropositive individuals are initially asymptomatic but typically develop AIDS related complex (ARC) followed by AIDS. Affected individuals exhibit severe immunosuppression which makes them highly susceptible to debilitating and ultimately fatal opportunistic infections. Replication of HIV by a host cell requires integration of the viral genome into the host cell's DNA. Since HIV is a retrovirus, the HIV replication cycle requires transcription of the viral RNA genome into DNA via an enzyme know as reverse transcriptase (RT).

Reverse transcriptase has three known enzymatic functions: The enzyme acts as an RNA-dependent DNA polymerase, as a ribonuclease, and as a DNA-dependent DNA polymerase. In its role as an RNA-dependent DNA polymerase, RT transcribes a single-stranded DNA copy of the viral RNA. As a ribonuclease, RT destroys the original viral RNA and frees the DNA just produced from the original RNA. And as a DNA-dependent DNA polymerase, RT makes a second, complementary DNA strand using the first DNA strand as a template. The two strands form double-stranded DNA, which is integrated into the host cell's genome by the integrase enzyme.

It is known that compounds that inhibit enzymatic functions of HIV RT will inhibit HIV replication in infected cells. These compounds are useful in the prophylaxis or treatment of HIV infection in humans. Among the compounds approved for use in treating HIV infection and AIDS are the RT inhibitors 3′-azido-3′-deoxythymidine (AZT), 2′,3′-dideoxyinosine (ddI), 2′,3′-dideoxycytidine (ddC), d4T, 3TC, nevirapine, delavirdine, efavirenz and abacavir.

While each of the foregoing drugs is effective in treating HIV infection and AIDS, there remains a need to develop additional HIV antiviral drugs including additional RT inhibitors. A particular problem is the development of mutant HIV strains that are resistant to the known inhibitors. The use of RT inhibitors to treat AIDS often leads to viruses that are less sensitive to the inhibitors. This resistance is typically the result of mutations that occur in the reverse transcriptase segment of the pol gene. The continued use of antiviral compounds to prevent HIV infection will inevitably result in the emergence of new resistant strains of HIV. Accordingly, there is a particular need for new RT inhibitors that are effective against mutant HIV strains.

The following references are of interest as background:

Williams et al., J. Med. Chem. 1993, vol. 36, pp. 1291-1294 discloses 5-chloro-3-(phenylsulfonyl)indole-2-carboxamide as a non-nucleoside inhibitor of HIV-1 reverse transcriptase.

Young et al., Bioorg. & Med. Chem. Letters 1995, vol. 5, pp. 491-496 discloses certain 2-heterocyclic indole-3-sulfones as inhibitors of HIV-1 reverse transcriptase.

GB 2,282,808 discloses certain 2-heterocyclic indole-3-sulfones as inhibitors of HIV reverse transcriptase and its resistant varieties.

U.S. Pat. No. 5,527,819 discloses certain 2-acyl substituted indole-3-sulfones as inhibitors of HIV reverse transcriptase.

WO 02/083216 A1 and WO 2004/014364 A1 each disclose certain substituted phenylindoles for the treatment of HIV.

### SUMMARY OF THE INVENTION

The present invention is directed to certain pyrrole-2,5-dicarboxamide compounds and their use in the inhibition of HIV reverse transcriptase, the prophylaxis of infection by HIV, the treatment of infection by HIV, and the prophylaxis, treatment, and delay in the onset or progression of AIDS and/or ARC. More particularly, the present invention includes compounds of Formula I and pharmaceutically acceptable salts thereof:

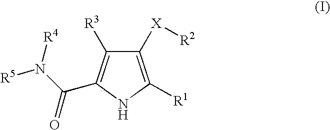

wherein:

### X is S, S(O), S(O)₂, P(O)—OT, P(S)—OT, or P(N—U)—OT;

T is H or independently has the same definition as R²;
U independently has the same definition as R^{K};

### R¹ is C(O)NR^{K}R^{L};

one of R^{K} and R^{L} is H, and the other of R^{K} and R^{L} is:
(1) H,
(2) C₁₋₆ alkyl,
(3) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(4) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(5) CycA,
(6) AryA,
(7) HetA,
(8) C₁₋₆ alkyl substituted with CycA, AryA, or HetA, or
(9) C₁₋₆ alkyl substituted with Y¹-CycA, Y¹-AryA, or Y¹-HetA;

### R² is:

(1) C₁₋₆ alkyl,
(3) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(4) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(3) CycB,
(4) AryB,
(5) HetB,
(6) C₁₋₆ alkyl substituted with CycB, AryB, or HetB,
(7) N(R^{A})R^{B},
(8) N(R^{A})—C₁₋₆ alkyl, wherein the alkyl is substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B}, with the proviso that OH, O—C₁₋₆ alkyl, or O—C₁₋₆ haloalkyl is not attached to the carbon in C₁₋₆ alkyl that is directly attached to the rest of the molecule,
(9) N(R^{A})-CycB,
(10) N(R^{A})-AryB,
(11) N(R^{A})-HetB,
(12) N(R^{A})—C₁₋₆ alkyl, wherein the alkyl is substituted with CycB, AryB, or HetB,
(13) C₂₋₆ alkenyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(14) C₂₋₆ alkenyl substituted with CycB, AryB, or HetB,
(15) C₂₋₆ alkynyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B}, or
(16) C₂₋₆ alkynyl substituted with CycB, AryB, or HetB;

### R³ is:

(1) H,
(2) halogen,
(3) C₁₋₆ alkyl,
(4) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(5) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(6) CycC,
(7) AryC,
(8) HetC,
(9) C₁₋₆ alkyl substituted with CycC, AryC, or HetC, or
(10) C₁₋₆ alkyl substituted with Y²-CycC, Y²-AryC, or Y²-HetC;

### R⁴ is:

(1) H,
(2) C₁₋₆ alkyl,
(3) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(4) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(5) CycD,
(6) AryD,
(7) HetD,
(8) C₁₋₆ alkyl substituted with CycD, AryD, or HetD, or
(9) C₁₋₆ alkyl substituted with Y³-CycD, Y³-AryD, or Y³-HetD;

### R⁵ is:

(1) C₁₋₆ alkyl,
(2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(4) CycE,
(5) AryE,
(6) HetE,
(7) C₁₋₆ alkyl substituted with CycE, AryE, or HetE, or
(8) C₁₋₆ alkyl substituted with Y⁴-CycE, Y⁴-AryE, or Y⁴-HetE;
   alternatively R⁴ and R⁵ together with the nitrogen atom to which they are both attached form:
(i) a 4- to 7-membered, saturated or unsaturated monocyclic ring optionally containing 1 or 2 heteroatoms in addition to the nitrogen attached to R⁴ and R⁵ selected from N, O, and S, where each S is optionally oxidized to S(O) or S(O)₂, or
(ii) a 7- to 12-membered bicyclic ring system wherein each ring in (ii) is independent of, fused to, or bridged with the other ring and each ring is saturated or unsaturated, and wherein the bicyclic ring system optionally contains from 1 to 3 heteroatoms in addition to the nitrogen attached to R⁴ and R⁵ selected from N, O, and S, where each S is optionally oxidized to S(O) or S(O)₂, and
wherein the monocyclic ring or the bicyclic ring system is optionally substituted with from 1 to 3 substituents each of which is independently:
   (1) C₁₋₆ alkyl,
   (2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
   (3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, or SO₂N(R^{A})R^{B},
   (4) O—C₁₋₆ alkyl,
   (5) O—C₁₋₆ haloalkyl,
   (6) OH,
   (7) oxo,
   (8) halogen,
   (9) CN,
   (10) NO₂,
   (11) N(R^{A})R^{B},
   (12) C(O)N(R^{A})R^{B},
   (13) C(O)R^{A},
   (14) C(O)—C₁₋₆ haloalkyl,
   (15) C(O)OR^{A},
   (16) OC(O)N(R^{A})R^{B},
   (17) SR^{A},
   (18) S(O)R^{A},
   (19) S(O)₂R^{A}, or
   (20) S(O)₂N(R^{A})R^{B};
      each R^{A} is independently H or C₁₋₆ alkyl;
      each R^{B} is independently H or C₁₋₆ alkyl;
      CycA is a carbocycle which is a C₃₋₈ cycloalkyl, a C₅₋₈ cycloalkenyl, or a C₇₋₁₂ bicyclic, saturated or unsaturated, non-aromatic ring system wherein one ring is fused to or bridged with the other ring; wherein the carbocycle is optionally substituted with a total of from 1 to 6 substituents, wherein:

(i) from zero to 6 substituents are each independently:
(1) halogen,
(2) CN
(3) C₁₋₆ alkyl,
(4) OH,
(5) O—C₁₋₆ alkyl,
(6) C₁₋₆ haloalkyl, or
(7) O—C₁₋₆ haloalkyl, and

(ii) from zero to 2 substituents are each independently:
(1) CycQ,
(2) AryQ,
(3) HetQ,
(4) HetR,
(4) Z-CycQ,
(5) Z-AryQ,
(6) Z-HetQ,
(7) Z-HetR, or
(7) C₁₋₆ alkyl substituted with CycQ, AryQ, HetQ, HetR, Z-CycQ, Z-AryQ, Z-HetQ, or Z-HetR;
   AryA is aryl which is optionally substituted with a total of from 1 to 8 substituents, wherein:

(i) from zero to 8 substituents are each independently:
(1) C₁₋₆ alkyl,
(2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(4) O—C₁₋₆ alkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) S(O)₂R^{A},
(19) S(O)₂N(R^{A})R^{B},
(20) N(R^{A})S(O)₂R^{B},
(21) N(R^{A})S(O)₂N(R^{A})R^{B},
(22) N(R^{A})C(O)R^{B},
(23) N(R^{A})C(O)N(R^{A})R^{B},
(24) N(R^{A})C(O)—C(O)N(R^{A})R^{B},
(25) N(R^{A})CO₂R^{B},
(26) C₂₋₆ alkenyl, or
(27) C₂₋₆ alkynyl, and

(ii) from zero to 2 substituents are each independently:
(1) CycQ,
(2) AryQ,
(3) HetQ,
(4) HetR,
(4) Z-CycQ,
(5) Z-AryQ,
(6) Z-HetQ,
(7) Z-HetR, or
(8) C₁₋₆ alkyl substituted with CycQ, AryQ, HetQ, HetR, Z-CycQ, Z-AryQ, Z-HetQ, or Z-HetR;
   HetA is a heterocycle which is optionally substituted with a total of from 1 to 8 substituents, wherein:

(i) from zero to 8 substituents are each independently:
(1) C₁₋₆ alkyl,
(2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(4) O—C₁₋₆ alkyl,
(5) O—C₁₋₆ haloalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)N(R^{A})R^{B},
(17) SR^{A},
(18) S(O)R^{A},
(19) S(O)₂R^{A},
(20) S(O)₂N(R^{A})R^{B},
(21) N(R^{A})S(O)₂R^{B},
(22) N(R^{A})S(O)₂N(R^{A})R^{B},
(23) N(R^{A})C(O)R^{B},
(24) N(R^{A})C(O)N(R^{A})R^{B},
(25) N(R^{A})C(O)—C(O)N(R^{A})R^{B}, or
(26) N(R^{A})CO₂R^{B}, and

(ii) from zero to 2 substituents are each independently:
(1) CycQ,
(2) AryQ,
(3) HetQ,
(4) HetR,
(4) Z-CycQ,
(5) Z-AryQ,
(6) Z-HetQ,
(7) Z-HetR, or
(7) C₁₋₆ alkyl substituted with CycQ, AryQ, HetQ, HetR, Z-CycQ, Z-AryQ, Z-HetQ, or Z-HetR;
   CycB, CycC, CycD and CycE each independently have the same definition as CycA;
   AryB, AryC, AryD and AryE each independently have the same definition as AryA;
   HetB, HetC, HetD and HetE each independently have the same definition as HetA;
   each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
   each heterocycle is independently (i) a 4- to 8-membered, saturated or unsaturated monocyclic ring, (ii) a 7- to 12-membered bicyclic ring system, or (iii) a 10- to 18-membered tricyclic ring system, wherein each ring in (ii) or (iii) is independent of, fused to, or bridged with the other ring or rings and each ring is saturated or unsaturated, and the monocyclic ring, bicyclic ring system, or tricyclic ring system contains from 1 to 8 heteroatoms selected from N, O and S and a balance of carbon atoms; and wherein any one or more of the nitrogen and sulfur heteroatoms is optionally oxidized, and any one or more of the nitrogen heteroatoms is optionally quaternized;
   Y¹, Y², Y³ and Y⁴ are each independently selected from the group consisting of:

(i) O,

(ii) S,

(iii) S(O),

(iv) S(O)₂,

(v) O—C₁₋₆ alkylene,

(vi) S—C₁₋₆ alkylene,

(vii) S(O)—C₁₋₆ alkylene,

(viii) S(O)₂—C₁₋₆ alkylene,

(ix) N(R^{A}),

(x) N(R^{A})—C₁₋₆ alkylene,

(xi) C(O),

(xii) C(O)—C₁₋₆ alkylene,

(xiii) C(O)—C₁₋₆ alkylene-O,

(xiv) C(O)N(R^{A}),

(xv) C(O)N(R^{A})—C₁₋₆ alkylene,

(xvi C(O)N(R^{A})—C₁₋₆ alkylene-C(O)O, and

(xvii) C(O)N(R^{A})S(O)₂;

each CycQ is independently C₃₋₈ cycloalkyl or C₅₋₈ cycloalkenyl, wherein the cycloalkyl or cycloalkenyl is optionally substituted with from 1 to 4 substituents, each of which is independently halogen, C₁₋₆ alkyl, OH, O—C₁₋₆ alkyl, C₁₋₆ haloalkyl, or O—C₁₋₆ haloalkyl; each AryQ is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B};
each HetQ is independently (i) a 5- or 6-membered heteroaromatic ring containing from 1 to 4 heteroatoms independently selected from N, O and S, wherein each N is optionally in the form of an oxide, or (ii) a 9- or 10-membered heterobicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂; and wherein the heteroaromatic ring or the heterobicyclic ring is optionally substituted with from 1 to 4 substituents each of which is independently halogen, C₁₋₆ alkyl, C₁₋₆ haloalkyl, OH, O—C₁₋₆ alkyl, O—C₁₋₆ haloalkyl, N(R^{A})R^{B}, C(O)N(R^{A})R^{B}, C(O)R^{A}, CO₂R^{A}, SO₂R^{A}, N(R^{A})C(O)N(R^{A})R^{B}, or N(R^{A})CO₂R^{B};
each HetR is independently a 4- to 7-membered, saturated or unsaturated, non-aromatic heterocyclic ring containing at least one carbon atom and from 1 to 4 heteroatoms independently selected from N, O and S, where each S is optionally oxidized to S(O) or S(O)₂, and wherein the saturated or unsaturated heterocyclic ring is optionally substituted with from 1 to 4 substituents each of which is independently halogen, CN, C₁₋₆ alkyl, OH, oxo, O—C₁₋₆ alkyl, C₁₋₆ haloalkyl, O—C₁₋₆ haloalkyl, C(O)N(R^{A})R^{B}, C(O)R^{A}, CO₂R^{A}, or SO₂R^{A}; and
each Z is independently:

(i) O,

(ii) S,

(iii) S(O),

(iv) S(O)₂,

(v) O—C₁₋₆ alkylene,

(vi) S—C₁₋₆ alkylene,

(vii) S(O)—C₁₋₆ alkylene,

(viii) S(O)₂—C₁₋₆ alkylene,

(ix) N(R^{A}), or

(x) N(R^{A})—C₁₋₆ alkylene.

Other embodiments, aspects and features of the present invention are either further described in or will be apparent from the ensuing description, examples and appended claims.

### DETAILED DESCRIPTION OF THE INVENTION

The compounds of Formula I above, and pharmaceutically acceptable salts thereof, are HIV reverse transcriptase inhibitors. The compounds are useful for inhibiting HIV reverse transcriptase and for inhibiting HIV replication in vitro and in vivo. More particularly, the compounds of Formula I inhibit the polymerase function of HIV-1 reverse transcriptase. Based upon the testing of representative compounds of the invention in the assays set forth in Examples 121 and 122 below, it is known that compounds of Formula I inhibit the RNA-dependent DNA polymerase activity of HIV-1 reverse transcriptase. Representative compounds of the present invention also exhibit activity against drug resistant forms of HIV (e.g., mutant strains of HIV in which reverse transcriptase has a mutation at lysine 103→asparagine (K103N) and/or tyrosine 181→cysteine (Y181C)), and thus can exhibit decreased cross-resistance against currently approved antiviral therapies.

A first embodiment of the present invention (alternatively referred to herein as “Embodiment E1”) is a compound of Formula I (alternatively and more simply referred to as “Compound I”), or a pharmaceutically acceptable salt thereof, wherein

### X is S, S(O), or S(O)₂;

### R² is:

(1) C₁₋₆ alkyl,
(3) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(4) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(3) CycB,
(4) AryB,
(5) HetB,
(6) C₁₋₆ alkyl substituted with CycB, AryB, or HetB,
(7) N(R^{A})R^{B},
(8) N(R^{A})—C₁₋₆ alkyl, wherein the alkyl is substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B}, with the proviso that OH, O—C₁₋₆ alkyl, or O—C₁₋₆ haloalkyl is not attached to the carbon in C₁₋₆ alkyl that is directly attached to the rest of the molecule,
(9) N(R^{A})-CycB,
(10) N(R^{A})-AryB,
(11) N(R^{A})—HetB, or
(12) N(R^{A})—C₁₋₆ alkyl, wherein the alkyl is substituted with CycB, AryB, or HetB; and
   AryA is aryl which is optionally substituted with a total of from 1 to 8 substituents, wherein:

(i) from zero to 8 substituents are each independently (1) C₁₋₆ alkyl, (2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A}, (3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B}, (4) O—C₁₋₆ alkyl, (5) O—C₁₋₆ haloalkyl, (6) OH, (7) halogen, (8) CN, (9) NO₂, (10) N(R^{A})R^{B}, (11) C(O)N(R^{A})R^{B}, (12) C(O)R^{A}, (13) C(O)—C₁₋₆ haloalkyl, (14) C(O)OR^{A}, (15) OC(O)N(R^{A})R^{B}, (16) SR^{A}, (17) S(O)R^{A}, (18) S(O)₂R^{A}, (19) S(O)₂N(R^{A})R^{B}, (20) N(R^{A})S(O)₂R^{B}, (21) N(R^{A})S(O)₂N(R^{A})R^{B}, (22) N(R^{A})C(O)R^{B}, (23) N(R^{A})C(O)N(R^{A})R^{B}, (24) N(R^{A})C(O)—C(O)N(R^{A})R^{B}, or (25) N(R^{A})CO₂R^{B}, and

(ii) from zero to 2 substituents are each independently (1) CycQ, (2) AryQ, (3) HetQ, (4) HetR, (4) Z-CycQ, (5) Z-AryQ, (6) Z-HetQ, (7) Z-HetR, or (8) C₁₋₆ alkyl substituted with CycQ, AryQ, HetQ, HetR, Z-CycQ, Z-AryQ, Z-HetQ, or Z-HetR;

AryB, AryC, AryD, and AryE each independently have the same definition as AryA;
and all other variables are as originally defined (i.e., as defined in the Summary of the Invention).

A second embodiment of the present invention (Embodiment E2) is a compound of Formula I, or a pharmaceutically acceptable salt thereof, wherein one of R^{K} and R^{L} is H, and the other of R^{K} and R^{L} is:
(1) H,
(2) C₁₋₆ alkyl,
(3) C₁₋₆ fluoroalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(4) C₁₋₆ alkyl substituted with 1 or 2 substituents each of which is independently OH, O—C₁₋₆ alkyl, O—C₁₋₆ fluoroalkyl, CN, C(O)N(R^{A})R^{B}, C(O)R^{A}, CO₂R^{A}, SR^{A}, S(O)R^{A}, SO₂R^{A}, or SO₂N(R^{A})R^{B},
(5) CycA,
(6) AryA,
(7) HetA, or
(8) C₁₋₆ alkyl substituted with CycA, AryA, or HetA; and all other variables are as originally defined or as defined in Embodiment E1.

A third embodiment of the present invention (Embodiment E3) is a compound of Formula I, or a pharmaceutically acceptable salt thereof, wherein one of R^{K} and R^{L} is H, and the other of R^{K} and R^{L} is:
(1) H,
(2) C₁₋₄ alkyl,
(3) C₁₋₄ fluoroalkyl, which is optionally substituted with O—C₁₋₄ alkyl or CO₂R^{A},
(4) C₁₋₄ alkyl substituted with O—C₁₋₄ alkyl, O—C₁₋₄ fluoroalkyl, C(O)R^{A}, CO₂R^{A}, or SO₂R^{A}, or
(5) C₁₋₄ alkyl substituted with CycA, AryA, or HetA;
   and all other variables are as originally defined or as defined in Embodiment E1.

A fourth embodiment of the present invention (Embodiment E4) is a compound of Formula I, or a pharmaceutically acceptable salt thereof, wherein R^{K} is H; R^{L} is H, C₁₋₄ alkyl, CH₂CF₃, CH₂CH₂CF₃, CH₂CF₂CF₃, CH(CO₂CH₃)CH₂CF₃, (CH₂)₂₋₃OCH₃, CH₂-AryA, or CH₂-HetA; and all other variables are as originally defined or as defined in Embodiment E1.

A fifth embodiment of the present invention (Embodiment E5) is a compound of Formula I, or a pharmaceutically acceptable salt thereof, wherein R² is AryB, HetB, N(R^{A})R^{B}, or N(R^{A})-CycB; and all other variables are as originally defined or as defined in any of the preceding embodiments.

A sixth embodiment of the present invention (Embodiment E6) is a compound of Formula I, or a pharmaceutically acceptable salt thereof, wherein R² is AryB, HetB, or N(R^{A})-CycB; and all other variables are as originally defined or as defined in any of the preceding embodiments.

A seventh embodiment of the present invention (Embodiment E7) is a compound of Formula I, or a pharmaceutically acceptable salt thereof, wherein R² is AryB or HetB; and all other variables are as originally defined or as defined in any of the preceding embodiments.

An eighth embodiment of the present invention (Embodiment E8) is a compound of Formula I, or a pharmaceutically acceptable salt thereof, wherein R³ is C₁₋₆ alkyl or CycC; and all other variables are as originally defined or as defined in any of the preceding embodiments.

A ninth embodiment of the present invention (Embodiment E9) is a compound of Formula I, or a pharmaceutically acceptable salt thereof, wherein R³ is C₁₋₄ alkyl; and all other variables are as originally defined or as defined in any of the preceding embodiments.

A tenth embodiment of the present invention (Embodiment E10) is a compound of Formula I, or a pharmaceutically acceptable salt thereof, wherein R³ is CH₃, CH₂CH₃, CH₂CH₂CH₃, CH(CH₃)₂, or CH₂CH₂CH₂CH₃; and all other variables are as originally defined or as defined in any of the preceding embodiments.

An eleventh embodiment of the present invention (Embodiment E11) is a compound of Formula I, or a pharmaceutically acceptable salt thereof, wherein R⁴ is H, C₁₋₆ alkyl, or C₁₋₆ alkyl substituted with CycD, AryD, or HetD; and all other variables are as originally defined or as defined in any of the preceding embodiments.

A twelfth embodiment of the present invention (Embodiment E12) is a compound of Formula I, or a pharmaceutically acceptable salt thereof, wherein R⁴ is H, C₁₋₄ alkyl, or C₁₋₄ alkyl substituted with AryD; and all other variables are as originally defined or as defined in any of the preceding embodiments.

A thirteenth embodiment of the present invention (Embodiment E13) is a compound of Formula I, or a pharmaceutically acceptable salt thereof, wherein R⁴ is H, CH₃, CH₂CH₃, or benzyl; and all other variables are as originally defined or as defined in any of the preceding embodiments.

A fourteenth embodiment of the present invention (Embodiment E14) is a compound of Formula I, or a pharmaceutically acceptable salt thereof, wherein R⁵ is C₁₋₆ alkyl substituted with AryE, O-AryE, or HetE; and all other variables are as originally defined or as defined in any of the preceding embodiments.

A fifteenth embodiment of the present invention (Embodiment E15) is a compound of Formula I, or a pharmaceutically acceptable salt thereof, wherein R⁵ is C₁₋₄ alkyl substituted with AryE, O-AryE, or HetE; and all other variables are as originally defined or as defined in any of the preceding embodiments.

A sixteenth embodiment of the present invention (Embodiment E16) is a compound of Formula I, or a pharmaceutically acceptable salt thereof, wherein R⁵ is CH₂-AryE, CH₂CH₂-AryE, CH(CH₃)-AryE, CH₂O-AryE, CH₂CH₂O-AryE, CH₂-HetE, or CH₂CH₂-HetE; and all other variables are as originally defined or as defined in any of the preceding embodiments.

A seventeenth embodiment of the present invention (Embodiment E17) is a compound of Formula I, or a pharmaceutically acceptable salt thereof, wherein, as an alternative to being separately and independently defined as set forth originally or as set forth in any of the preceding embodiments, R⁴ and R⁵ together with the nitrogen atom to which they are both attached form a 4- to 7-membered, saturated ring optionally containing 1 heteroatom in addition to the nitrogen attached to R⁴ and R⁵ selected from N, O, and S, where the optional S is optionally oxidized to S(O) or S(O)₂; wherein the saturated ring is optionally fused to a benzene ring or a 5- or 6-membered heteroaromatic ring containing a heteroatom selected from N, O and S; and wherein the optionally fused saturated ring is optionally substituted with 1 to 3 substituents each of which is independently C₁₋₆ alkyl, OH, oxo, O—C₁₋₆ alkyl, C₁₋₆ fluoroalkyl, O—C₁₋₆ fluoroalkyl, C(O)N(R^{A})R^{B}, C(O)R^{A}, CO₂R^{A}, or SO₂R^{A}; and all other variables are as originally defined or as defined in any of the preceding embodiments.

An eighteenth embodiment of the present invention (Embodiment E18) is a compound of Formula I, or a pharmaceutically acceptable salt thereof, wherein, as an alternative to being separately and independently defined as set forth originally or as set forth in any of the preceding embodiments, R⁴ and R⁵ together with the nitrogen atom to which they are both attached form a heterocyclic ring optionally having a benzo or thieno ring fused thereto, which is selected from the group consisting of 1-azetidinyl 1-pyrrolidinyl, 1-piperidinyl, 1-piperazinyl, 1-azepanyl, 4-morpholinyl, 4-thiomorpholinyl, 3,4-dihydroisoquinolin-2(1H)-yl, 1,3,4,5-tetrahydro-2H-2-benzazepin-2-yl and 4,6,7,8-tetrahydro-5H-thieno[3,2-c]azepin-5-yl; wherein the optionally fused heterocyclic ring is optionally substituted with 1 or 2 substituents each of which is independently C₁₋₄ alkyl, OH, oxo, halogen, O—C₁₋₄ alkyl, or SO₂—C₁₋₄ alkyl; and all other variables are as originally defined or as defined in any of the preceding embodiments.

A nineteenth embodiment of the present invention (Embodiment E19) is a compound of Formula I, or a pharmaceutically acceptable salt thereof, wherein R⁴ and R⁵ are as defined in Embodiment E18 except that the optionally fused heterocyclic ring is optionally substituted with 1 or 2 substituents each of which is independently C₁₋₄ alkyl, OH, or oxo; and all other variables are as originally defined or as defined in any of the preceding embodiments.

A twentieth embodiment of the present invention (Embodiment E20) is a compound of Formula I, or a pharmaceutically acceptable salt thereof, wherein, as an alternative to being separately and independently defined as set forth originally or as set forth in any of the preceding embodiments, R⁴ and R⁵ together with the nitrogen atom to which they are both attached form a heterocyclic ring optionally having a benzo or thieno ring fused thereto, which is selected from the group consisting of 1-azetidinyl, 1-pyrrolidinyl, 1-piperidinyl, 1-piperazinyl, 1-azepanyl, 4-morpholinyl, 4-thiomorpholinyl, 3,4-dihydroisoquinolin-2(1H)-yl, 1,3,4,5-tetrahydro-2H-2-benzazepin-2-yl and 4,6,7,8-tetrahydro-5H-thieno[3,2-c]azepin-5-yl; wherein the optionally fused heterocyclic ring is optionally substituted with 1 or 2 substituents each of which is independently CH₃, OH, oxo, Cl, Br. F, OCH₃, or SO₂CH₃; and all other variables are as originally defined or as defined in any of the preceding embodiments.

A twenty-first embodiment of the present invention (Embodiment E21) is a compound of Formula I, or a pharmaceutically acceptable salt thereof, wherein R⁴ and R⁵ are as defined in Embodiment E20 except that the optionally fused heterocyclic ring is optionally substituted with 1 or 2 substituents each of which is independently CH₃, OH, or oxo; and all other variables are as originally defined or as defined in any of the preceding embodiments.

A twenty-second embodiment of the present invention (Embodiment E22) is a compound of Formula I, or a pharmaceutically acceptable salt thereof, wherein X is S(O)₂; and all other variables are as originally defined or as defined in any of the preceding embodiments.

A twenty-third embodiment of the present invention (Embodiment E23) is a compound of Formula I, or a pharmaceutically acceptable salt thereof, wherein CycA is C₃₋₆ cycloalkyl which is optionally substituted with a total of from 1 to 3 substituents each of which is independently fluorine, C₁₋₆ alkyl, OH, O—C₁₋₆ alkyl, C₁₋₆ fluoroalkyl, or O—C₁₋₆ fluoroalkyl; and all other variables are as originally defined or as defined in any of the preceding embodiments.

A twenty-fourth embodiment of the present invention (Embodiment E24) is a compound of Formula I, or a pharmaceutically acceptable salt thereof, wherein CycA is C₃₋₆ cycloalkyl which is optionally substituted with 1 or 2 substituents each of which is independently C₁₋₄ alkyl, OH, O—C₁₋₄ alkyl, C₁₋₄ fluoroalkyl, or O—C₁₋₄ fluoroalkyl; and all other variables are as originally defined or as defined in any of the preceding embodiments.

A twenty-fifth embodiment of the present invention (Embodiment E25) is a compound of Formula I, or a pharmaceutically acceptable salt thereof, wherein CycA is C₃₋₆ cycloalkyl; and all other variables are as originally defined or as defined in any of the preceding embodiments.

A twenty-sixth embodiment of the present invention (Embodiment E26) is a compound of Formula I, or a pharmaceutically acceptable salt thereof, wherein AryA is phenyl or naphthyl, wherein the phenyl or naphthyl is optionally substituted with a total of from 1 to 6 substituents wherein:

(i) from zero to 6 substituents are each independently:
(1) C₁₋₆ alkyl,
(2) C₁₋₆ fluoroalkyl,
(3) C₁₋₆ alkyl substituted with OH, O—C₁₋₄ alkyl, O—C₁₋₄ haloalkyl, CN, N(R^{A})R^{B}, C(O)N(R^{A})R^{B}, C(O)R^{A}, CO₂R^{A}, SR^{A}, S(O)R^{A}, SO₂R^{A}, or SO₂N(R^{A})R^{B},
(4) O—C₁₋₆ alkyl,
(5) O—C₁₋₆ fluoroalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₄ fluoroalkyl,
(14) CO₂R^{A},
(15) SR^{A},
(16) S(O)R^{A},
(17) SO₂R^{A}, or
(18) SO₂N(R^{A})R^{B}, and

(ii) from zero to 1 substituent is independently:
(1) CycQ,
(2) AryQ,
(3) HetQ, or
(4) C₁₋₆ alkyl substituted with CycQ, AryQ, or HetQ; and all other variables are as originally defined or as defined in the any of the preceding embodiments.

A twenty-seventh embodiment of the present invention (Embodiment E27) is a compound of Formula I, or a pharmaceutically acceptable salt thereof, wherein AryA is phenyl or naphthyl, wherein the phenyl is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A}; and all other variables are as originally defined or as defined in any of the preceding embodiments.

A twenty-eighth embodiment of the present invention (Embodiment E28) is a compound of Formula I, or a pharmaceutically acceptable salt thereof, wherein AryA is phenyl which is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A}; and all other variables are as originally defined or as defined in any of the preceding embodiments.

A twenty-ninth embodiment of the present invention (Embodiment E29) is a compound of Formula I, or a pharmaceutically acceptable salt thereof, wherein AryA is phenyl which is optionally substituted with from 1 to 3 substituents each of which is independently CH₃, CF₃, OCH₃, OCF₃, OH, Cl, Br, F, CN, NO₂, NH₂, N(H)CH₃, N(CH₃)₂, C(O)NH₂, C(O)N(H)CH₃, C(O)N(CH₃)₂, C(O)CH₃, C(O)CF₃, CO₂CH₃, or SO₂CH₃; and all other variables are as originally defined or as defined in any of the preceding embodiments.

A thirtieth embodiment of the present invention (Embodiment E30) is a compound of Formula I, or a pharmaceutically acceptable salt thereof, wherein HetA is a heteroaryl which is (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂, wherein the heteroaryl is optionally substituted with a total of from 1 to 6 substituents, wherein:

(i) from zero to 6 substituents are each independently:
(1) C₁₋₆ alkyl,
(2) C₁₋₆ fluoroalkyl,
(3) C₁₋₆ alkyl substituted with OH, O—C₁₋₄ alkyl, O—C₁₋₄ haloalkyl, CN, N(R^{A})R^{B}, C(O)N(R^{A})R^{B}, C(O)R^{A}, CO₂R^{A}, SR^{A}, S(O)R^{A}, SO₂R^{A}, or SO₂N(R^{A})R^{B},
(4) O—C₁₋₆ alkyl,
(5) O—C₁₋₆ fluoroalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₄ fluoroalkyl,
(15) CO₂R^{A},
(16) SR^{A},
(17) S(O)R^{A},
(18) SO₂R^{A}, or
(19) SO₂N(R^{A})R^{B}, and

(ii) from zero to 1 substituent is independently:
(1) CycQ,
(2) AryQ,
(3) HetQ, or
(4) C₁₋₆ alkyl substituted with CycQ, AryQ, or HetQ;
   and all other variables are as originally defined or as defined in any of the preceding embodiments.

A thirty-first embodiment of the present invention (Embodiment E31) is a compound of Formula I, or a pharmaceutically acceptable salt thereof, wherein HetA is a heteroaryl selected from the group consisting of thienyl, furanyl, pyrrolyl, imidazolyl, pyrazolyl, triazolyl, tetrazolyl, oxazolyl, isooxazolyl, thiazolyl, isothiazolyl, oxadiazolyl, thiadiazolyl, pyridinyl, pyrazinyl, pyrimidinyl, pyridazinyl, benzofuranyl, benzothienyl, indolyl, indazolyl, isobenzofuranyl, benzisoxazolyl, benzoxazolyl, benzimidazolyl, benzopiperidinyl, chromenyl, quinolinyl, isoquinolinyl, cinnolinyl, quinoxalinyl, quinazolinyl, and imidazopyridinyl, wherein the heteroaryl is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A}; and all other variables are as originally defined or as defined in any of the preceding embodiments.

A thirty-second embodiment of the present invention (Embodiment E32) is a compound of Formula I, or a pharmaceutically acceptable salt thereof, wherein HetA is a heteroaryl selected from the group consisting of pyridinyl, pyrrolyl, thienyl, furanyl, imidazolyl, pyrazolyl, triazolyl, tetrazolyl, oxazolyl, isooxazolyl, oxadiazolyl, thiazolyl, isothiazolyl, thiadiazolyl, benzofuranyl, benzothienyl, indolyl, indazolyl, isobenzofuranyl, benzoxazolyl, benzimidazolyl, quinolinyl, isoquinolinyl, wherein the heteroaryl is optionally substituted with from 1 to 3 substituents each of which is independently CH₃, CF₃, OCH₃, OCF₃, OH, Cl, Br, F, CN, C(O)NH₂, C(O)N(H)CH₃, C(O)N(CH₃)₂, C(O)CH₃, C(O)CF₃, CO₂CH₃, or SO₂CH₃; and all other variables are as originally defined or as defined in any of the preceding embodiments.

A thirty-third embodiment of the present invention (Embodiment E33) is a compound of Formula I, or a pharmaceutically acceptable salt thereof, wherein CycB independently has the definition as set forth for CycA in Embodiment E23 or Embodiment E24 or Embodiment E25; and all other variables are as originally defined or as defined in any of the preceding embodiments.

A thirty-fourth embodiment of the present invention (Embodiment E34) is a compound of Formula I, or a pharmaceutically acceptable salt thereof, wherein CycB is C₃₋₆ cycloalkyl which is optionally substituted with 1 or 2 substituents each of which is independently C₁₋₄ alkyl, OH, O—C₁₋₄ alkyl, C₁₋₄ fluoroalkyl, or O—C₁₋₄ fluoroalkyl; and all other variables are as originally defined or as defined in any of the preceding embodiments.

A thirty-fifth embodiment of the present invention (Embodiment E35) is a compound of Formula I, or a pharmaceutically acceptable salt thereof, wherein CycB is C₃₋₆ cycloalkyl; and all other variables are as originally defined or as defined in any of the preceding embodiments.

A thirty-sixth embodiment of the present invention (Embodiment E36) is a compound of Formula I, or a pharmaceutically acceptable salt thereof, wherein AryB independently has the definition as set forth for AryA in Embodiment E26 or Embodiment E27 or Embodiment E28 or Embodiment E29; and all other variables are as originally defined or as defined in any of the preceding embodiments.

A thirty-seventh embodiment of the present invention (Embodiment E37) is a compound of Formula I, or a pharmaceutically acceptable salt thereof, wherein AryB is phenyl or naphthyl, wherein the phenyl is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A}; and all other variables are as originally defined or as defined in any of the preceding embodiments.

A thirty-eighth embodiment of the present invention (Embodiment E38) is a compound of Formula I, or a pharmaceutically acceptable salt thereof, wherein AryB is phenyl or naphthyl, wherein the phenyl is optionally substituted with from 1 to 3 substituents each of which is independently CH₃, CF₃, OCH₃, OCF₃, OH, Cl, Br, F, CN, NO₂, NH₂, N(H)CH₃, N(CH₃)₂, C(O)NH₂, C(O)N(H)CH₃, C(O)N(CH₃)₂, C(O)CH₃, C(O)CF₃, CO₂CH₃, or SO₂CH₃; and all other variables are as originally defined or as defined in any of the preceding embodiments.

A thirty-ninth embodiment of the present invention (Embodiment E39) is a compound of Formula I, or a pharmaceutically acceptable salt thereof, wherein HetB independently has the definition as set forth for HetA in Embodiment E30 or Embodiment E31 or Embodiment E32; and all other variables are as originally defined or as defined in any of the preceding embodiments.

A fortieth embodiment of the present invention (Embodiment E40) is a compound of Formula I, or a pharmaceutically acceptable salt thereof, wherein HetB is a 4- to 7-membered saturated heterocyclic ring optionally containing from 1 to 3 heteroatoms selected from 1 to 3 N atoms, zero or 1 O atom, and zero or 1 S atom, wherein the ring is attached to the rest of the compound via a N atom and the optional S atom is optionally oxidized to S(O) or S(O)₂, and wherein the saturated heterocyclic ring is optionally substituted with 1 to 3 substituents each of which is independently C₁₋₆ alkyl, oxo, C(O)N(R^{A})R^{B}, C(O)R^{A}, CO₂R^{A} or S(O)₂R^{A}; and all other variables are as originally defined or as defined in any of the preceding embodiments.

A forty-first embodiment of the present invention (Embodiment E41) is a compound of Formula I, or a pharmaceutically acceptable salt thereof, wherein HetB is a saturated heterocyclic ring selected from the group consisting of azetidinyl, pyrrolidinyl, piperidinyl, morpholinyl, thiomorpholinyl, piperazinyl, thiazinanyl, thiazepanyl and azepanyl, wherein the ring is attached to the rest of the compound via a ring nitrogen atom, and wherein the ring is optionally substituted with from 1 to 3 substituents each of which is independently C₁₋₄ alkyl or oxo; and all other variables are as originally defined or as defined in any of the preceding embodiments.

A forty-second embodiment of the present invention (Embodiment E42) is a compound of Formula I, or a pharmaceutically acceptable salt thereof, wherein HetB is a saturated heterocyclic ring selected from the group consisting of:

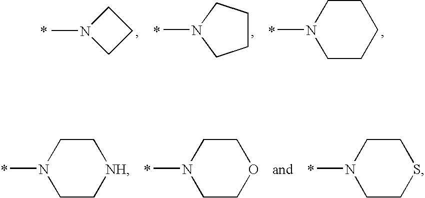

wherein the asterisk * denotes the point of attachment to the rest of the compound, and wherein the ring is optionally substituted with 1 or 2 substituents each of which is CH₃ or oxo; and all other variables are as originally defined or as defined in any of the preceding embodiments.

A forty-third embodiment of the present invention (Embodiment E43) is a compound of Formula I, or a pharmaceutically acceptable salt thereof, wherein CycC independently has the definition as set forth for CycA in Embodiment E23 or Embodiment E24 or Embodiment E25; and all other variables are as originally defined or as defined in any of the preceding embodiments.

A forty-fourth embodiment of the present invention (Embodiment E44) is a compound of Formula I, or a pharmaceutically acceptable salt thereof, wherein AryC independently has the definition as set forth for AryA in Embodiment E26 or Embodiment E27 or Embodiment E28 or Embodiment E29; and all other variables are as originally defined or as defined in any of the preceding embodiments.

A forty-fifth embodiment of the present invention (Embodiment E45) is a compound of Formula I, or a pharmaceutically acceptable salt thereof, wherein HetC independently has the definition as set forth for HetA in Embodiment E30 or Embodiment E31 or Embodiment E32; and all other variables are as originally defined or as defined in any of the preceding embodiments.

A forty-sixth embodiment of the present invention (Embodiment E46) is a compound of Formula I, or a pharmaceutically acceptable salt thereof, wherein CycD independently has the definition as set forth for CycA in Embodiment E23 or Embodiment E24 or Embodiment E25; and all other variables are as originally defined or as defined in any of the preceding embodiments.

A forty-seventh embodiment of the present invention (Embodiment E47) is a compound of Formula I, or a pharmaceutically acceptable salt thereof, wherein AryD independently has the definition as set forth for AryA in Embodiment E26 or Embodiment E27 or Embodiment E28 or Embodiment E29; and all other variables are as originally defined or as defined in any of the preceding embodiments.

A forty-eighth embodiment of the present invention (Embodiment E48) is a compound of Formula I, or a pharmaceutically acceptable salt thereof, wherein AryD is phenyl which is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A}; and all other variables are as originally defined or as defined in any of the preceding embodiments.

A forty-ninth embodiment of the present invention (Embodiment E49) is a compound of Formula I, or a pharmaceutically acceptable salt thereof, wherein HetD independently has the definition as set forth for HetA in Embodiment E30 or Embodiment E31 or Embodiment E32; and all other variables are as originally defined or as defined in any of the preceding embodiments.

A fiftieth embodiment of the present invention (Embodiment E50) is a compound of Formula I, or a pharmaceutically acceptable salt thereof, wherein HetD is independently an optionally substituted heteroaryl as defined for HetA in Embodiment E30 or is a 4- to 7-membered, saturated heterocyclic ring containing 1 or 2 heteroatoms selected from N, O, and S, where each S is optionally oxidized to S(O) or S(O)₂, wherein the saturated ring is optionally substituted with 1 to 3 substituents each of which is independently C₁₋₆ alkyl, OH, oxo, O—C₁₋₆ alkyl, C₁₋₆ fluoroalkyl, O—C₁₋₆ fluoroalkyl, C(O)R^{A}, CO₂R^{A}, or SO₂R^{A}; and all other variables are as originally defined or as defined in any of the preceding embodiments.

A fifty-first embodiment of the present invention (Embodiment E51) is a compound of Formula I, or a pharmaceutically acceptable salt thereof, wherein CycE independently has the definition as set forth for CycA in Embodiment E23 or Embodiment E24 or Embodiment E25; and all other variables are as originally defined or as defined in any of the preceding embodiments.

A fifty-second embodiment of the present invention (Embodiment E52) is a compound of Formula I, or a pharmaceutically acceptable salt thereof, wherein AryE independently has the definition as set forth for AryA in Embodiment E26 or Embodiment E27 or Embodiment E28 or Embodiment E29; and all other variables are as originally defined or as defined in any of the preceding embodiments.

A fifty-third embodiment of the present invention (Embodiment E53) is a compound of Formula I, or a pharmaceutically acceptable salt thereof, wherein AryE is phenyl which is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A}; and all other variables are as originally defined or as defined in any of the preceding embodiments.

A fifty-fourth embodiment of the present invention (Embodiment E54) is a compound of Formula I, or a pharmaceutically acceptable salt thereof, wherein AryE is phenyl which is optionally substituted with from 1 to 3 substituents each of which is independently CH₃, CF₃, OCH₃, OCF₃, OH, Cl, Br, F, CN, NO₂, NH₂, N(H)CH₃, N(CH₃)₂, C(O)NH₂, C(O)N(H)CH₃, C(O)N(CH₃)₂, C(O)CH₃, C(O)CF₃, CO₂CH₃, or SO₂CH₃; and all other variables are as originally defined or as defined in any of the preceding embodiments.

A fifty-five embodiment of the present invention (Embodiment E55) is a compound of Formula I, or a pharmaceutically acceptable salt thereof, wherein HetE independently has the definition as set forth for HetA in Embodiment E30 or Embodiment E31 or Embodiment E32; and all other variables are as originally defined or as defined in any of the preceding embodiments.

A fifty-sixth embodiment of the present invention (Embodiment E56) is a compound of Formula I, or a pharmaceutically acceptable salt thereof, wherein HetE independently has the definition as set forth for HetD in Embodiment E50; and all other variables are as originally defined or as defined in any of the preceding embodiments.

A fifty-seventh embodiment of the present invention (Embodiment E57) is a compound of Formula I, or a pharmaceutically acceptable salt thereof, wherein HetE is independently:
(i) a heteroaryl selected from the group consisting of thienyl, furanyl, pyrrolyl, imidazolyl, pyrazolyl, triazolyl, tetrazolyl, oxazolyl, isooxazolyl, thiazolyl, isothiazolyl, oxadiazolyl, thiadiazolyl, pyridinyl, pyrazinyl, pyrimidinyl, pyridazinyl, benzofuranyl, benzothienyl, indolyl, indazolyl, isobenzofuranyl, benzisoxazolyl, benzoxazolyl, benzimidazolyl, benzopiperidinyl, chromenyl, quinolinyl, isoquinolinyl, cinnolinyl, quinoxalinyl, quinazolinyl, and imidazopyridinyl, wherein the heteroaryl is (a) optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A}, and (b) additionally and optionally substituted with phenyl, or
(ii) a saturated heterocyclic ring selected from the group consisting of azetidinyl, pyrrolidinyl, piperidinyl, morpholinyl, thiomorpholinyl, piperazinyl, thiazinanyl, thiazepanyl and azepanyl, wherein the ring is attached to the rest of the compound via a ring carbon atom, and wherein the ring is optionally substituted with 1 to 3 substituents each of which is independently C₁₋₄ alkyl or oxo;
   and all other variables are as originally defined or as defined in any of the preceding embodiments.

A fifty-eighth embodiment of the present invention (Embodiment E58) is a compound of Formula I, or a pharmaceutically acceptable salt thereof, wherein HetE is independently:
(i) a heteroaryl selected from the group consisting of thienyl, furanyl, pyrrolyl, imidazolyl, pyrazolyl, triazolyl, tetrazolyl, oxazolyl, isooxazolyl, thiazolyl, isothiazolyl, oxadiazolyl, thiadiazolyl, pyridinyl, pyrazinyl, pyrimidinyl, pyridazinyl, benzofuranyl, benzothienyl, indolyl, indazolyl, isobenzofuranyl, benzisoxazolyl, benzoxazolyl, benzimidazolyl, benzopiperidinyl, chromenyl, quinolinyl, isoquinolinyl, cinnolinyl, quinoxalinyl, quinazolinyl, and imidazopyridinyl, wherein the heteroaryl is (a) optionally substituted with from 1 to 3 substituents each of which is independently CH₃, CF₃, OCH₃, OCF₃, OH, Cl, Br, F, CN, NO₂, NH₂, N(H)CH₃, N(CH₃)₂, C(O)NH₂, C(O)N(H)CH₃, C(O)N(CH₃)₂, C(O)CH₃, C(O)CF₃, CO₂CH₃, or SO₂CH₃, and (b) additionally and optionally substituted with phenyl, or
(ii) a saturated heterocyclic ring selected from the group consisting of azetidinyl, pyrrolidinyl, piperidinyl, morpholinyl, thiomorpholinyl, piperazinyl, thiazinanyl, thiazepanyl and azepanyl, wherein the ring is attached to the rest of the compound via a ring carbon atom, and wherein the ring is optionally substituted with 1 or 2 substituents each of which is CH₃ or oxo;
   and all other variables are as originally defined or as defined in any of the preceding embodiments.

A fifty-ninth embodiment of the present invention (Embodiment E59) is a compound of Formula I, or a pharmaceutically acceptable salt thereof, wherein each CycQ is independently C₃₋₆ cycloalkyl which is optionally substituted with 1 or 2 substituents, each of which is independently fluorine, C₁₋₆ alkyl, OH, O—C₁₋₆ alkyl, C₁₋₆ fluoroalkyl, or O—C₁₋₆ fluoroalkyl; and all other variables are as originally defined or as defined in any of the preceding embodiments.

A sixtieth embodiment of the present invention (Embodiment E60) is a compound of Formula I, or a pharmaceutically acceptable salt thereof, wherein each AryQ is independently phenyl which is optionally substituted with from 1 to 3 substituents each of which is independently halogen, CN, NO₂, C₁₋₆ alkyl, C₁₋₆ fluoroalkyl, OH, O—C₁₋₆ alkyl, O—C₁₋₆ fluoroalkyl, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B}; and all other variables are as originally defined or as defined in any of the preceding embodiments.

A sixty-first embodiment of the present invention (Embodiment E61) is a compound of Formula I, or a pharmaceutically acceptable salt thereof, wherein each HetQ is independently a 5- or 6-membered heteroaromatic ring containing from 1 to 4 heteroatoms independently selected from N, O and S, wherein each N is optionally in the form of an oxide, wherein the heteroaromatic ring is optionally substituted with a total of from 1 to 4 substituents each of which is independently halogen, C₁₋₆ alkyl, C₁₋₆ fluoroalkyl, OH, O—C₁₋₆ alkyl, O—C₁₋₆ fluoroalkyl, N(R^{A})R^{B}, (C)ON(R^{A})R^{B}, C(O)R^{A}, CO₂R^{A}, SO₂R^{A}, N(R^{A})C(O)N(R^{A})R^{B}, or N(R^{A})CO₂R^{B}; and all other variables are as originally defined or as defined in any of the preceding embodiments.

A sixty-second embodiment of the present invention (Embodiment E62) is a compound of Formula I, or a pharmaceutically acceptable salt thereof, wherein R^{A} and R^{B} are each independently H or C₁₋₄ alkyl; and all other variables are as originally defined or as defined in any of the preceding embodiments.

A sixty-third embodiment of the present invention (Embodiment E63) is a compound of Formula I, or a pharmaceutically acceptable salt thereof, wherein R^{A} and R^{B} are each independently H or C₁₋₃ alkyl; and all other variables are as originally defined or as defined in any of the preceding embodiments.

A sixty-fourth embodiment of the present invention (Embodiment E64) is a compound of Formula I, or a pharmaceutically acceptable salt thereof, wherein R^{A} and R^{B} are each independently H or CH₃; and all other variables are as originally defined or as defined in any of the preceding embodiments.

A sixty-fifth embodiment of the present invention (Embodiment E65) is a compound of Formula I, or a pharmaceutically acceptable salt thereof, wherein Y¹, Y², Y³ and Y⁴ are each independently selected from the group consisting of:

(i) O,

(ii) S,

(iii) S(O),

(iv) S(O)₂,

(v) OCH₂,

(vi) SCH₂,

(vii) S(O)CH₂,

(viii) S(O)₂CH₂,

(ix) N(R^{A}),

(x) N(R^{A})CH₂,

(xi) C(O),

(xii) C(O)CH₂,

(xiii) C(O)CH₂O,

(xiv) C(O)N(R^{A}),

(xv) C(O)N(R^{A})CH₂,

(xvi C(O)N(R^{A})CH₂C(O)O, and

(xvii) C(O)N(R^{A})S(O)₂;

and all other variables are as originally defined or as defined in any of the preceding embodiments.

A sixty-sixth embodiment of the present invention (Embodiment E66) is a compound of Formula I, or a pharmaceutically acceptable salt thereof, wherein each Z is independently:

(i) O,

(ii) S,

(iii) S(O),

(iv) S(O)₂,

(v) OCH₂,

(vi) SCH₂,

(vii) S(O)CH₂,

(viii) S(O)₂CH₂,

(ix) N(R^{A}), or

(x) N(R^{A})CH₂;

and all other variables are as originally defined or as defined in any of the preceding embodiments.

A sixty-seventh embodiment of the present invention (Embodiment E67) is a compound selected from the group consisting of the compounds set forth in Examples 1 to 119 below (including Examples 70A and 70B) and their pharmaceutically acceptable salts. A first aspect of Embodiment E67 is a compound selected from the group consisting of the compounds set forth in Examples 1 to 82 (excluding Examples 70A and 70B) and 116 to 119 below and their pharmaceutically acceptable salts. A second aspect of Embodiment E67 is a compound selected from the group consisting of the compounds set forth in Examples 70A, 70B and 83 to 115 below and their pharmaceutically acceptable salts.

stopped

A first class of compounds of the present invention (alternatively referred to herein as Class C1) includes compounds of Formula I and pharmaceutically acceptable salts thereof, wherein:

R^{K} and R^{L} are as defined in Embodiment E2;

CycA is as defined in Embodiment E23;

AryA is as defined in Embodiment E26;

HetA is as defined in Embodiment E30;

R² is as defined in Embodiment E4;

CycB is as defined in Embodiment E33;

AryB is as defined in Embodiment E36;

HetB is as defined in Embodiment E40;

R³ is as defined in Embodiment E8;

CycC independently has the same definition as CycA;

R⁴ is as defined in Embodiment E11;

CycD independently has the same definition as CycA;

AryD independently has the same definition as AryA;

HetD is as defined in Embodiment E50;

R⁵ is as defined in Embodiment E14;

CycE independently has the same definition as CycA;

AryE independently has the same definition as AryA;

CycD independently has the same definition as CycA;

HetE independently has the same definition as HetD;

R⁴ and R⁵ are together alternatively as defined in Embodiment E17;

CycQ is as defined in Embodiment E59;

AryQ is as defined in Embodiment E60; and

HetQ is as defined in Embodiment E61;

and all other variables are as originally defined.

A second class of compounds of the present invention (Class C2) includes compounds of Formula I and pharmaceutically acceptable salts thereof, wherein:

X is as defined in Embodiment E22;

R^{K} and R^{L} are as defined in Embodiment E3;

CycA is as defined in Embodiment E24;

AryA is as defined in Embodiment E28;

HetA is as defined in Embodiment E31;

R² is as defined in Embodiment E6;

CycB is as defined in Embodiment E34;

AryB is as defined in Embodiment E37;

HetB is as defined in Embodiment E41;

R³ is as defined in Embodiment E9;

R⁴ is as defined in Embodiment E12;

AryD independently is as defined in Embodiment E48;

R⁵ is as defined in Embodiment E15;

AryE is as defined in Embodiment E53;

HetE is as defined in Embodiment E57;

R⁴ and R⁵ are together alternatively as defined in Embodiment E18; and

R^{A} and R^{B} are as defined in Embodiment E62.

A first subclass of the second class of compounds of the present invention (Subclass SC2-1) includes compounds of Formula I and pharmaceutically acceptable salts thereof, wherein R⁴ and R⁵ are together alternatively as defined in Embodiment E19; and all other variables are as originally defined in Class C2.

A third class of compounds of the present invention (Class C3) includes compounds of Formula I and pharmaceutically acceptable salts thereof, wherein:

X is as defined in Embodiment E22;

R^{K} and R^{L} are as defined in Embodiment E4;

AryA is as defined in Embodiment E29;

HetA is as defined in Embodiment E32;

R² is as defined in Embodiment E7;

CycB is as defined in Embodiment E35;

AryB is as defined in Embodiment E38;

HetB is as defined in Embodiment E42;

R³ is as defined in Embodiment E10;

R⁴ is as defined in Embodiment E13;

R⁵ is as defined in Embodiment E16;

AryE is as defined in Embodiment E54;

HetE is as defined in Embodiment E58; and

R⁴ and R⁵ are together alternatively as defined in Embodiment E20.

A first subclass of the third class of compounds of the present invention (Subclass SC3-1) includes compounds of Formula I and pharmaceutically acceptable salts thereof, wherein R⁴ and R⁵ are together alternatively as defined in Embodiment E21; and all other variables are as originally defined in Class C3.

Another embodiment of the present invention is a compound of Formula I, or a pharmaceutically acceptable salt thereof, as originally defined or as defined in any of the foregoing embodiments, aspects, classes, or sub-classes, wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt % to 100 wt. %), and most preferably at least about 99 wt. % (e.g., 100 wt. %) of a product containing a compound Formula I or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest impurity level governs. A compound or salt of 100% purity is one which is free of detectable impurities as determined by a standard method of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

Other embodiments of the present invention include the following:

(a) A pharmaceutical composition comprising an effective amount of a compound of Formula I as defined above, or a pharmaceutically acceptable salt thereof, and a pharmaceutically acceptable carrier.

(b) A pharmaceutical composition which comprises the product prepared by combining (e.g., mixing) an effective amount of a compound of Formula I as defined above, or a pharmaceutically acceptable salt thereof, and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, HIV reverse transcriptase inhibitors other than a compound of Formula I, HIV integrase inhibitors, HIV fusion inhibitors, and HIV entry inhibitors.

(e) The pharmaceutical composition of (d), wherein the anti-HIV agent is an antiviral selected from the group consisting of HIV protease inhibitors, HIV reverse transcriptase inhibitors other than a compound of Formula I, and HIV integrase inhibitors.

(f) A combination which is (i) a compound of Formula I as defined above, or a pharmaceutically acceptable salt thereof, and (ii) another anti-HIV agent selected from the group consisting of HIV antiviral agents, immunomodulators, and anti-infective agents; wherein Compound I and the anti-HIV agent are each employed in an amount that renders the combination effective for inhibition of HIV reverse transcriptase, for treatment or prophylaxis of infection by HIV, or for treatment, prophylaxis of, or delay in the onset or progression of AIDS.

(g) The combination of (f), wherein the other anti-HIV agent is an antiviral selected from the group consisting of HIV protease inhibitors, HIV reverse transcriptase inhibitors, HIV integrase inhibitors, HIV fusion inhibitors, and HIV entry inhibitors.

(h) The combination of (g), wherein the other anti-HIV agent is an antiviral selected from the group consisting of HIV protease inhibitors, HIV reverse transcriptase inhibitors, and HIV integrase inhibitors.

(i) A method for the inhibition of HIV reverse transcriptase in a subject in need thereof which comprises administering to the subject an effective amount of a compound of Formula I.

(j) A method of the prophylaxis or treatment of infection by HIV (e.g., HIV-1) in a subject in need thereof which comprises administering to the subject an effective amount of a compound of Formula I.

(k) The method of (j), wherein the compound of Formula I is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, and HIV entry inhibitors.
(l) The method of (j), wherein the compound of Formula I is administered in combination with an effective amount of at least one other HIV antiviral selected from the group consisting of HIV protease inhibitors, HIV integrase inhibitors, non-nucleoside HIV reverse transcriptase inhibitors, and nucleoside HIV reverse transcriptase inhibitors.

(m) A method for the prophylaxis, treatment or delay in the onset or progression of AIDS in a subject in need thereof which comprises administering to the subject an effective amount of a compound of Formula I.

(n) The method of (m), wherein the compound is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, and HIV entry inhibitors.

(o) The method of (m), wherein the compound is administered in combination with an effective amount of at least one other HIV antiviral selected from the group consisting of HIV protease inhibitors, HIV integrase inhibitors, non-nucleoside HIV reverse transcriptase inhibitors, and nucleoside HIV reverse transcriptase inhibitors.

(p) A method for the inhibition of HIV reverse transcriptase in a subject in need thereof which comprises administering to the subject the pharmaceutical composition of (a), (b), (c), (d) or (e) or the combination of (f), (g) or (h).

(q) A method for the prophylaxis or treatment of infection by HIV (e.g., HIV-1) in a subject in need thereof which comprises administering to the subject the pharmaceutical composition of (a), (b), (c), (d) or (e) or the combination of (f), (g) or (h).

(r) A method for the prophylaxis, treatment, or delay in the onset or progression of AIDS in a subject in need thereof which comprises administering to the subject the pharmaceutical composition of (a), (b), (c), (d) or (e) or the combination of (f), (g) or (h).

The present invention also includes a compound of Formula I, or a pharmaceutically acceptable salt thereof, (i) for use in, (ii) for use as a medicament for, or (iii) for use in the preparation or manufacture of a medicament for: (a) inhibition of HIV reverse transcriptase, (b) treatment or prophylaxis of infection by HIV, or (c) treatment, prophylaxis of, or delay in the onset or progression of AIDS. In these uses, the compounds of the present invention can optionally be employed in combination with one or more anti-HIV agents selected from HIV antiviral agents, anti-infective agents, and immunomodulators.

Additional embodiments of the invention include the pharmaceutical compositions, combinations and methods set forth in (a)-(r) above and the uses set forth in the preceding paragraph, wherein the compound of the present invention employed therein is a compound of one of the embodiments, aspects, classes or subclasses described above. In all of these embodiments, the compound may optionally be used in the form of a pharmaceutically acceptable salt.

Additional embodiments of the present invention include each of the pharmaceutical compositions, combinations, methods and uses set forth in the preceding paragraphs, wherein the compound of the present invention or its salt employed therein is substantially pure. With respect to a pharmaceutical composition comprising a compound of Formula I or its salt and a pharmaceutically acceptable carrier and optionally one or more excipients, it is understood that the term “substantially pure” is in reference to a compound of Formula I or its salt per se; i.e., the purity of the active ingredient in the composition.

The present invention also includes prodrugs of the compounds of Formula I. The term “prodrug” refers to a derivative of a compound of Formula I, or a pharmaceutically acceptable salt thereof, which is converted in vivo into Compound I. Prodrugs of compounds of Formula I can exhibit enhanced solubility, absorption, and/or lipophilicity compared to the compounds per se, thereby resulting in increased bioavailability and efficacy. The in vivo conversion of the prodrug can be the result of an enzyme-catalyzed chemical reaction, a metabolic chemical reaction, and/or a spontaneous chemical reaction (e.g., solvolysis). When the compound contains, for example, a hydroxy group, the prodrug can be a derivative of the hydroxy group such as an ester (—OC(O)R), a carbonate ester (—OC(O)OR), a phosphate ester (—O—P(═O)(OH)₂), or an ether (—OR). Other examples include the following: When the compound of Formula I contains a carboxylic acid group, the prodrug can be an ester or an amide, and when the compound of Formula I contains a primary amino group or another suitable nitrogen that can be derivatized, the prodrug can be an amide, carbamate, urea, imine, or a Mannich base. One or more functional groups in Compound I can be derivatized to provide a prodrug thereof. Conventional procedures for the selection and preparation of suitable prodrug derivatives are described, for example, in Design of Prodrugs, edited by H. Bundgaard, Elsevier, 1985; J. J. Hale et al., J. Med. Chem. 2000, vol. 43, pp. 1234-1241; C. S. Larsen and J. Ostergaard, “Design and application of prodrugs” in: Textbook of Drug Design and Discovery, 3^{rd} edition, edited by C. S. Larsen, 2002, pp. 410-458; and Beaumont et al., Current Drug Metabolism 2003, vol. 4, pp. 461-458; the disclosures of each of which are incorporated herein by reference in their entireties.

As used herein, the term “alkyl” refers to any monovalent straight or branched chain, saturated aliphatic hydrocarbon radical having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and iso-propyl, ethyl and methyl. As another example, “C₁₋₄ alkyl” refers to n-, iso-, sec- and t-butyl, n- and isopropyl, ethyl and methyl.

The term “alkylene” refers to any divalent linear or branched chain aliphatic hydrocarbon radical having a number of carbon atoms in the specified range. Thus, for example, “—C₁₋₆ alkylene-” refers to any of the C₁ to C₆ linear or branched alkylenes, and “—C₁₋₄ alkylene-” refers to any of the C₁ to C₄ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Another sub-class of interest is an alkylene selected from the group consisting of —CH₂—, —CH(CH₃)—, and —C(CH₃)₂—.

The term “alkenyl” refers to a monovalent straight or branched chain aliphatic hydrocarbon radical containing one carbon-carbon double bond and having a number of carbon atoms in the specified range. One class of alkenyls are those having 2 to 6 carbon atoms. A preferred class of alkenyls are those having 2 to 4 carbon atoms. Examples of alkenyl groups are vinyl (ethenyl), 2-propenyl, isopropenyl, and isobutenyl.

The term “alkynyl” refers to a monovalent straight or branched chain aliphatic hydrocarbon radical containing one carbon-carbon triple bond and having a number of carbon atoms in the specified range. One class of alkynyls are those having 2 to 6 carbon atoms. A preferred class of alkynyls are those having 2 to 4 carbon atoms. Examples of alkynyl groups are ethynyl and propynyl.

The term “cycloalkyl” refers to any monocyclic ring of an alkane having a number of carbon atoms in the specified range. Thus, for example, “C₃₋₈ cycloalkyl” (or “C₃-C₈ cycloalkyl”) refers to cyclopropyl, cyclobutyl, cyclopentyl, cyclohexyl, cycloheptyl, and cyclooctyl.

The term “cycloalkenyl” refers to any monocyclic ring of an alkene having a number of carbon atoms in the specified range. Thus, for example, “C₃₋₈ cycloalkenyl” (or “C₃-C₈ cycloalkenyl”) refers to cyclopropyl, cyclobutenyl, cyclopentenyl, cyclohexenyl, cycloheptenyl, or cyclooctenyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms have been replaced with a halogen (i.e., F, Cl, Br and/or I). Thus, for example, “C₁₋₆ haloalkyl” (or “C haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.). A fluoroalkyl of particular interest is CF₃.

The term “C(O)” refers to carbonyl. The terms “S(O)₂” and “SO₂” each refer to sulfonyl. The term “S(O)” refers to sulfinyl.

The left-most atom or variable shown in any of the groups in the definitions of R¹ to R⁵ is the atom or variable attached to the rest of the molecule. Thus, for example, a definition equivalent to R^{1═}C(O)NR^{K}R^{L} is R¹═*—C(O)NR^{K}R^{L}. As another example the definition of a compound of the present invention in which R¹ is C(O)NR^{K}R^{L}, R^{K} is H, R^{L} is C₁₋₆ alkyl substituted with Y¹-CycA in which the C₁₋₆ alkyl is methyl and Y¹ is O—C₁₋₆ alkylene in which the C₁₋₆ alkylene is methylene (i.e., Y¹ is OCH₂), and R⁵ is C₁₋₆ alkyl substituted with Y⁴-AryE in which the C₁₋₆ alkyl is methyl and Y⁴ is N(R^{A})—C₁₋₆ alkylene in which the C₁₋₆ alkylene is methylene (i.e., Y⁴ is N(R^{A})—CH₂) is as follows:

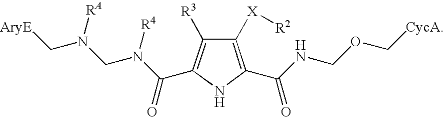

An asterisk (“*”) as the end of an open bond in a chemical group denotes the point of attachment of the group to the rest of the molecule

The term “carbocycle” refers to a monocyclic ring, a bicyclic ring system, or a polycyclic ring system in which all of the ring atoms are carbon atoms. One class of carbocycles of interest with respect to the invention includes the C₃₋₈ cycloalkyls, the C₅₋₈ cycloalkenyls, or the C₇₋₁₂ bicyclic, saturated or unsaturated, non-aromatic ring systems wherein one ring is fused to or bridged with the other ring. Representative members of this class of carbocycles are cyclopropyl, cyclobutyl, cyclopentyl, cyclohexyl, cycloheptyl, cyclooctyl, cyclopentenyl, cyclohexenyl, cycloheptenyl, octahydro-1H-indenyl, and decahydronaphthyl (decalinyl).

Aryls are another class of carbocycles of interest. The term “aryl” refers to (i) phenyl, (ii) 9- or 10-membered bicyclic, fused carbocylic ring systems in which at least one ring is aromatic, and (iii) 11- to 14-membered tricyclic, fused carbocyclic ring systems in which at least one ring is aromatic. Suitable aryls include, for example, phenyl, naphthyl, tetrahydronaphthyl (tetralinyl), indenyl, anthracenyl, and fluorenyl.

The term “heterocycle” refers to (i) a 4- to 8-membered, saturated or unsaturated monocyclic ring, (ii) a 7- to 12-membered bicyclic ring system, or (iii) a 10- to 18-membered tricyclic ring system, wherein each ring in (ii) or (iii) is independent of, fused to, or bridged with the other ring or rings and each ring is saturated or unsaturated, and the monocyclic ring, bicyclic ring system, or tricyclic ring system contains from 1 to 8 heteroatoms selected from N, O and S and a balance of carbon atoms; and wherein any one or more of the nitrogen and sulfur heteroatoms is optionally oxidized, and any one or more of the nitrogen heteroatoms is optionally quaternized. Suitable monocyclic rings include saturated heterocyclyls such as azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, dioxanyl, and azacyclooctyl. Suitable monocylic rings also include unsaturated heterocyclic rings such as those corresponding to the saturated heterocyclic rings listed in the preceding sentence in which a single bond is replaced with a double bond (e.g., a carbon-carbon single bond is replaced with a carbon-carbon double bond). Suitable ring systems include, for example, 7-azabicyclo[2.2.1]heptyl, decahydronaphthyridinyl, and decahydroquinolinyl.

The heterocycles include heteroaryls. The term “heteroaryl” refers to (i) 5- and 6-membered heteroaromatic rings and (ii) 9- and 10-membered bicyclic, fused ring systems in which at least one ring is aromatic, wherein the heteroaromatic ring or the bicyclic, fused ring system contains from 1 to 4 heteroatoms independently selected from N, O and S, wherein each N is optionally in the form of an oxide and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl

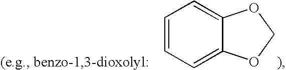

benzopiperidinyl, benzisoxazolyl, benzoxazolyl, chromanyl, isochromanyl, benzothienyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, and 2,3-dihydrobenzo-1,4-dioxinyl

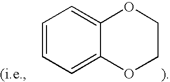

It is understood that the specific rings and ring systems suitable for use in the present invention are not limited to those listed in the preceding paragraphs. These rings and ring systems are merely representative.

Unless expressly stated to the contrary in a particular context, any of the various cyclic rings and ring systems contained herein may be attached to the rest of the compound at any ring atom (i.e., any carbon atom or any heteroatom) provided that a stable compound results.

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. As another example, an aryl or heteroaryl described as optionally substituted with “from 1 to 8 substituents” is intended to include as aspects thereof, an aryl or heteroaryl optionally substituted with 1 to 7 substituents, 1 to 6 substituents, 1 to 5 substituents, 1 to 4 substituents, 1 to 3 substituents, 1 to 2 substituents, 2 to 8 substituents, 2 to 7 substituents, 2 to 6 substituents, 2 to 5 substituents, 2 to 4 substituents, 2 to 3 substituents, 3 to 8 substituents, 3 to 7 substituents, 3 to 6 substituents, 3 to 5 substituents, 3 to 4 substituents, 4 to 8 substituents, 4 to 7 substituents, 4 to 6 substituents, 4 to 5 substituents, 5 to 8 substituents, 6 to 8 substituents, 7 to 8 substituents, 1 substituent, 2 substituents, 3 substituents, 4 substituents, 5 substituents, 6 substituents, 7 substituents, and 8 substituents.

When any variable (e.g., R^{A} or R^{B}) occurs more than one time in any constituent or in Formula I or in any other formula depicting and describing compounds of the present invention, its definition on each occurrence is independent of its definition at every other occurrence. Also, combinations of substituents and/or variables are permissible only if such combinations result in stable compounds.

Unless expressly stated to the contrary, substitution by a named substituent is permitted on any atom in a ring (e.g., cycloalkyl, aryl, or heteroaryl) provided such ring substitution is chemically allowed and results in a stable compound.

As a result of the selection of substituents and substituent patterns, certain compounds of the present invention can exhibit keto-enol tautomerism. All tautomeric forms of these compounds, whether individually or in mixtures, are within the scope of the present invention. Compounds of the present invention having a hydroxy substituent on a carbon atom of a heteroaromatic ring such that keto-enol tautomerism can occur are understood to include compounds in which only the hydroxy is present, compounds in which only the tautomeric keto form (i.e., an oxo substitutent) is present, and compounds in which the keto and enol forms are both present.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present invention are limited to stable compounds embraced by Formula I.

As a result of the selection of substituents and substituent patterns, certain compounds of the present invention can have asymmetric centers and can occur as mixtures of stereoisomers, or as individual diastereomers, or enantiomers. All isomeric forms of these compounds, whether individually or in mixtures, are within the scope of the present invention.

The methods of the present invention involve the use of compounds of the present invention in the inhibition of HIV reverse transcriptase (wild type and/or mutant strains thereof), the prophylaxis or treatment of infection by human immunodeficiency virus (HIV) and the prophylaxis, treatment or delay in the onset or progression of consequent pathological conditions such as AIDS. Prophylaxis of AIDS, treating AIDS, delaying the onset or progression of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery. As another example, the present invention can also be employed to prevent transmission of HIV from a pregnant female infected with HIV to her unborn child or from an HIV-infected female who is nursing (i.e., breast feeding) a child to the child via administration of an effective amount of Compound I or a pharmaceutically acceptable salt thereof.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. When compounds employed in the present invention carry an acidic moiety (e.g., —COOH or a phenolic group), suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of Formula I mean providing the compound or a prodrug of the compound to the individual in need of treatment or prophylaxis. When a compound or a prodrug thereof is provided in combination with one or more other active agents (e.g., antiviral agents useful for treating or prophylaxis of HIV infection or AIDS), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV reverse transcriptase (wild type and/or mutant strains thereof) and thereby elicit the response being sought (i.e., an “inhibition effective amount”). When the active compound (i.e., active ingredient) is administered as the salt, references to the amount of active ingredient are to the free form (i.e., the non-salt form) of the compound.

In the method of the present invention (i.e., inhibiting HIV reverse transcriptase, treating or prophylaxis of HIV infection or treating, prophylaxis of, or delaying the onset or progression of AIDS), the compounds of Formula I, optionally in the form of a salt/hydrate, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmacy, 21st edition, Lippincott Williams & Wilkins, 2005.

The compounds of Formula I can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to use of a compound of Formula I with one or more anti-HIV agents. An “anti-HIV agent” is any agent which is directly or indirectly effective in the inhibition of HIV reverse transcriptase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS, such as those disclosed in Table 1 of WO 01/38332 or in the Table in WO 02/30930. Suitable HIV antivirals for use in combination with the compounds of the present invention include, for example, those listed in Table A as follows:

TABLE A

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| efavirenz, EFV, Sustiva ®, Stocrin ® | nnRTI |
| efavirenz + emtricitabine + tenofovir DF, Atripla ® | nnRTI + nRTI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| PPL-100 (also known as PL-462) (Ambrilia) | PI |
| raltegravir, MK-0518, Isentress ™ | InI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, | nRTI |
| Viread ® | |
| tipranavir, Aptivus ® | PI |

EI = entry inhibitor;
FI = fusion inhibitor;
InI = integrase inhibitor;
PI = protease inhibitor;
nRTI = nucleoside reverse transcriptase inhibitor;
nnRTI = non-nucleoside reverse transcriptase inhibitor.
Some of the drugs listed in the table are used in a salt form; e.g., abacavir sulfate, indinavir sulfate, atazanavir sulfate, nelfinavir mesylate.

It is understood that the scope of combinations of the compounds of this invention with anti-HIV agents is not limited to the HIV antivirals listed in Table A and/or listed in the above-referenced Tables in WO 01/38332 and WO 02/30930, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), or the 59^{th} edition (2005). The dosage ranges for a compound of the invention in these combinations are the same as those set forth above.

Abbreviations employed herein include the following: AcOH=acetic acid; AIDS=acquired immunodeficiency syndrome; Bn=benzyl; BOC or Boc=t-butyloxycarbonyl; BrdUTP=bromodeoxyuridine triphosphate; n-Bu=n-butyl; t-Bu=t-butyl; CHAPS=3-[(3-cholamidopropyl)-dimethylammonio]-1-propane-sulfonate; DCC=dicyclohexyl carbodiimide; DCE=1,2-dichloroethane; DCM=dichloromethane; dGTP=deoxyguanosine triphosphate; DMF=dimethylformamide; dNTP=deoxynucleoside triphosphate; EDC=1-ethyl-3-(3-dimethylaminopropyl) carbodiimide; EDTA=ethylenediaminetetracetic acid; EGTA=ethylene glycol bis(2-aminoethyl ether)-N,N,N′,N′-tetraacetic acid; Et=ethyl; Et₃N=triethylamine; EtOH=ethanol; FBS=fetal bovine serum; HOBt=1-hydroxy benzotriazole; HPLC=high-performance liquid chromatography; i-Pr=isopropyl; MS=mass spectroscopy; n-Pr=n-propyl; TEA=triethylamine; TFA=trifluoroacetic acid; TfOH=triflic acid (=trifluoromethanesulfonic acid); THF=tetrahydrofuran.

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

Scheme 1 depicts the synthesis of a 4-arylsulfonyl-1H-pyrrole-2,5-dicarboxamides. The starting point is the pyrrole 1-1, which is commercially available or can be prepared in the manner described in Kleinspehn, J. Am. Chem. Soc. 1955, 77: 1546-48. In Scheme 1, pyrrole 1-1 is reacted with an aryldisulfide in the presence of a base (e.g., sodium hydride) in a polar aprotic solvent (e.g., dimethylformamide or dimethylsulfoxide) to provide arylthiopyrrole 1-2 (c.f., the analogous reaction with indole in Atkinson et al., Synthesis 1988, 480-81). Alternatively, pyrrole 1 can be reacted with an aryl sulfenyl chloride in a non-reactive anhydrous solvent (e.g., methylene chloride or chloroform) to give compound 1-2 (Fischer et al. Justus Liebigs Ann. Chem. 1928, 461: 244-77). The sulfide in 1-2 can be oxidized to the sulfone 1-3 with a variety of oxidizing agents including peracids and peroxides using an inertr organic solvent such as chloroform or methylene chloride under aqueous acid, base or buffered conditions. This is followed by oxidation of the 5-methyl substituent [e.g., reaction with (i) ceric ammonium nitrate (Paine et al., Canadian Journal of Chemistry, 1976, 54(3): 411-14) or (ii) chlorination with sulfuryl chloride in an inert solvent (e.g., a halohydrocarbon such as dichloroethane, chloroform or methylene chloride) followed by aqueous hydrolysis (Corwin et al., J. Am. Chem. Soc. 1942, 64: 1267-73)] to provide the aldehyde 1-4, which can be further oxidized to the corresponding carboxylic acid 1-5 with sodium chlorite, potassium permanganate, or chromic acid and an aqueous or organic solvent. Acid 1-5 can then be converted to an activated ester in dimethylformamide or methylene chloride using a carbodiimide coupling reagent like EDC or DCC and hydroxybenzotriazole, and coupled to an amine using standard coupling conditions, to give primary or secondary amides 1-6. The remaining ester in 1-6 can then be hydrolyzed to carboxylic acid 1-7 using an aqueous base with or without one or more organic co-solvents; or in the case of an acid sensitive ester, using acidic conditions (e.g., trifluoroacetic acid in methylene chloride or chloroform); or in the case of a benzyl ester, using catalytic hydrogenation to form the carboxylic acid. Conversion to dicarboxamide 1-9 can then be accomplished through the acid chloride 1-8 by reaction with the appropriate amine in the presence of a base and using a halogenated solvent, tetrahydrofuran, ethyl acetate or acetone. Alternatively an active ester can be formed in situ from 1-7 in dimethylformamide or methylene chloride using a carbodiimide coupling reagent like EDC or DCC and hydroxybenzotriazole or similar reagent, followed by reaction with the appropriate amine.

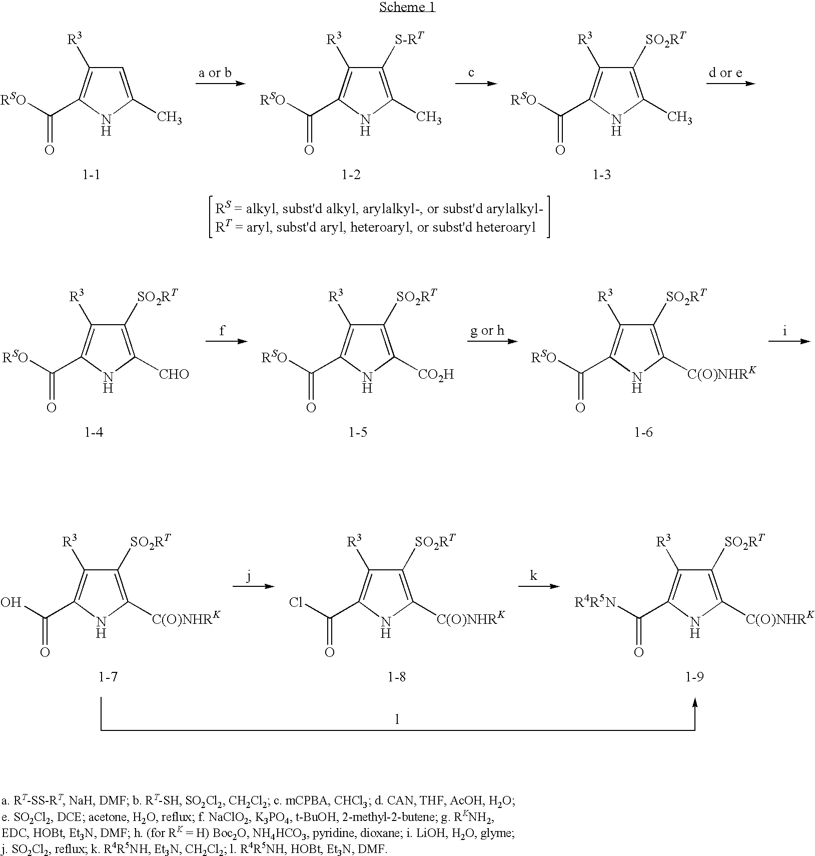

Scheme 2 provides an alternative method for preparing pyrroles of Formula I-1. This method is particularly useful for preparing pyrroles that are not commercially available and cannot be prepared in the manner described in Kleinspehn, J. Am. Chem. Soc. 1955, 77: 1546-48. Acylation of Meldrum's acid 2-1 with an acid chloride and an amine base (e.g., pyridine) provides 2-2, which after solvolysis with an alcohol in an inert solvent (e.g., an aromatic hydrocarbon such as benzene or toluene) and refluxing to effect decarboxylation gives the beta-keto ester 2-3 (Oikawa et al, J. Org. Chem. 1978, 43(10): 2087-88). Conversion of 2-3 to pyrrole 2-4 can be accomplished under the conditions of the Knorr synthesis using elevated temperatures (MacDonald, J. Chem. Soc. 1952: 4176-4182). Degradation of the benzyl ester to 2-5 can then be accomplished by selective conversion to the carboxylic acid by catalytic hydrogenation with a transition metal catalyst (e.g., palladium) in a suitable solvent (e.g., methanol, ethanol, isopropanol or ethyl acetate). This is followed by iodo-decarboxylation using iodine and an iodide salt in an aqueous, mildly basic solvent mixture at elevated temperatures to provide 2-6. Reductive dehalogenation of 2-6 using a transition metal catalyst (e.g., Pd) in an alcohol solvent (e.g., MeOH) in the presence of an amine base gives 1-1. Application of the chemistry of Scheme 1 to 1-1 affords the desired 1-9.

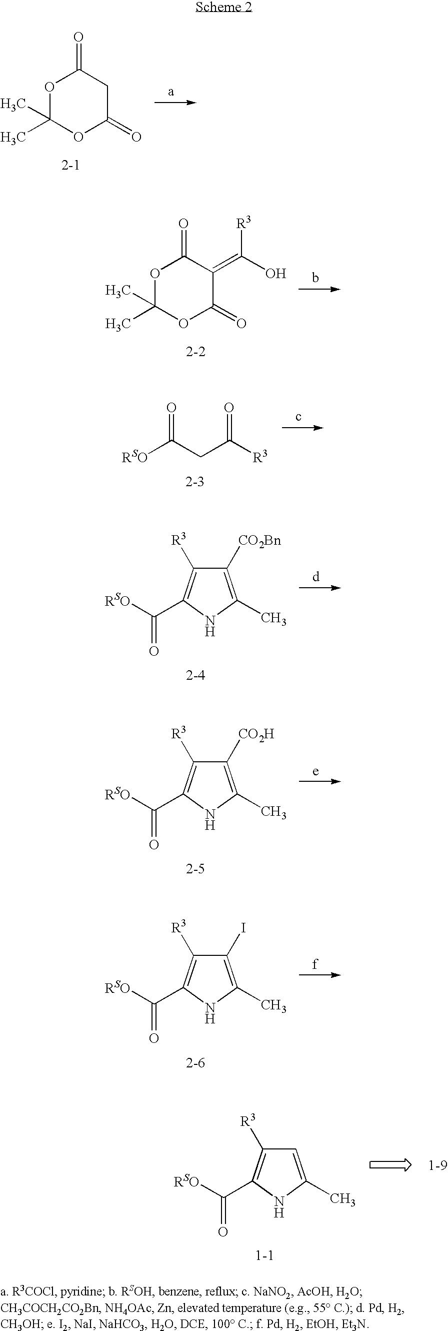

Scheme 3 depicts the preparation of 4-aminosulfonyl-1H-pyrrole-2,5-dicarboxamides, wherein pyrrole 1-1 can be made via a 1,3-diketone condensation/cyclization using dialkyl aminomalonate hydrochloride 3-1 and a beta-diketone, similar to the procedure developed by Paine and Dolphin, J. Org. Chem. 1985, 50: 2763-72. Subsequent sulfonylation of pyrrole 1-1 with neat chlorosulfonic acid affords the 4-chlorosulfonyl pyrrole 3-2. The chlorine in 3-2 can be displaced with a secondary amine in a non-protic solvent (e.g., methylene chloride, ethyl acetate, acetone or dimethylformamide) and a amine base (e.g., triethylamine, Hunig's base, or pyridine) to generate sulfonamide 3-3. The α-methyl group can be selectively oxidized to the carboxaldehyde 3-4 by dihalogenation with sulfuryl chloride in methylene chloride, chloroform, dichloroethane or another inert solvent, followed by hydrolysis with water using a co-solvent such as acetone, ethyl acetate, tetrahydrofuran or dioxane. The carboxaldehyde 3-4 can be further oxidized with sodium chlorite, potassium permanganate, or chromic acid in aqueous or organic solvent to the carboxylic acid 3-5, which can be converted to the primary or secondary amide 3-6 under peptide coupling conditions. The ester can then be hydrolyzed at elevated temperature in aqueous base (e.g., a metal hydroxide such as LiOH) in aqueous ether (e.g., 1,2-dimethoxyethane, tetrahydrofuran, or dioxane) or aqueous alcohol to afford 3-7. The carboxylic acid 3-7 can then be converted to the desired 3-8 using standard peptide coupling conditions as described above.

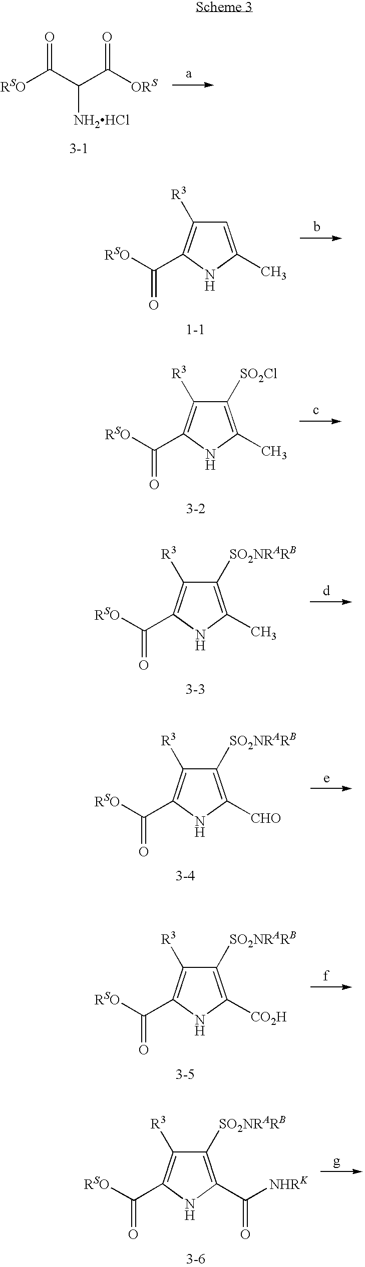

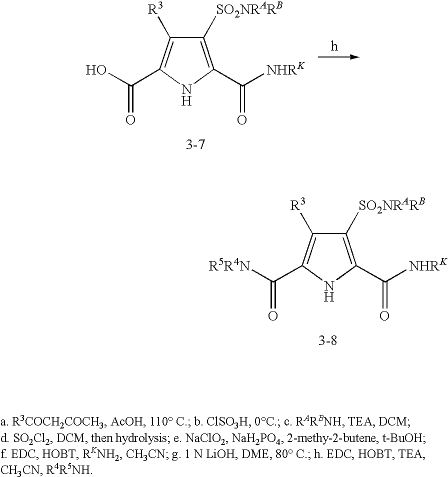

Scheme 4 depicts an alternative route to the 4-arylsulfonyl-1H-pyrrole-2,5-dicarboxamides of Scheme 1, wherein the route employs an indium(III) catalysis method described by Garzya et al., Tet. Letters 2004, 45: 1499-1501 for aryl sulfonylation of benzene sulfonyl chlorides. In the scheme, the pyrrole sulfonyl chloride intermediate 3-2 can be converted using indium(III) chloride catalysis to heteroaryl- or aryl-pyrrolylsulfone 1-3 often at lower temperatures and with shorter reaction times than enumerated in Garzya et al. From pyrrole 1-3, the conditions described in Scheme 1 can be employed to reach the desired 1-9.

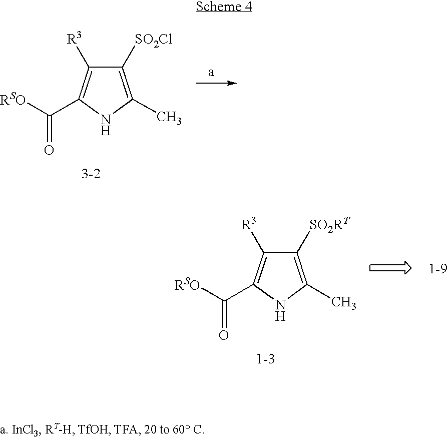

In the methods for preparing compounds of the present invention set forth in the foregoing schemes, functional groups in various moieties and substituents (in addition to those already explicitly noted in the foregoing schemes) may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2^{nd} edition, 1991. The protecting groups may be removed at a convenient subsequent stage using methods known in the art. Alternatively the interfering group can be introduced into the molecule subsequent to the reaction step of concern.

The following examples serve only to illustrate the invention and its practice. The examples are not to be construed as limitations on the scope or spirit of the invention.

### Example 1

### N-(2,4-Dichlorobenzyl)-N,3-dimethyl-4-(1-phenylsulfonyl)-1H-pyrrole-2,5-dicarboxamide

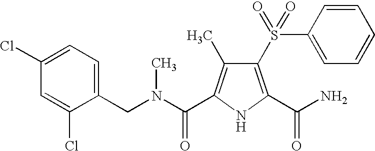

### Step 1: Ethyl 3,5-dimethyl-4-phenylthio-1H-pyrrole-2-carboxylate

A solution of ethyl 3,5-dimethyl-1H-pyrrole-2-carboxylate (5.00 g, 29.90 mmol) was dissolved in dry DMF (100 mL) in a 500 mL flask under nitrogen. Sodium hydride (1.43 g, 59.80 mmol, 60% dispersion in oil) was added and the reaction stirred at room temperature for 5 minutes. Benzene disulfide was added and the resulting mixture was stirred at 65° C. for 22 hours. The reaction mixture was poured into cold water (1 L), and the resulting solid was collected by filtration. The solid was suspended in hexane (500 mL), stirred for 10 minutes and then filtered. The solid was washed with hexane (100 mL) and dried to obtain the title compound.

### Step 2: Ethyl 3,5-dimethyl-4-phenylsulfonyl-1H-pyrrole-2-carboxylate

A solution of ethyl 3,5-dimethyl-4-phenylthio-1H-pyrrole-2-carboxylate (1.12 g, 4.06 mmol) in chloroform was cooled to 0° C. and 3-chloroperoxybenzoic acid (1.75 g, 10.16 mmol) was added. The reaction was stirred at room temperature overnight. The reaction mixture was diluted with methylene chloride and washed with saturated sodium bicarbonate (3×) and saturated sodium chloride. The organic phase was dried over sodium sulfate, filtered and the solvent evaporated to give the title compound.

### Step 3: Ethyl 5-formyl-3-methyl-4-phenylsulfonyl-1H-pyrrole-2-carboxylate

Ethyl 3,5-dimethyl-4-phenylsulfonyl-1H-pyrrole-2-carboxylate (0.500 g, 1.65 mmol) was dissolved in a solution of tetrahydrofuran (19 mL), acetic acid (23 mL) and water (19 mL). Ceric ammonium nitrate (3.56 g, 6.50 mmol) was added and the reaction stirred overnight at room temperature.

The mixture was poured into water (500 ml) and extracted with methylene chloride. The organic solution was washed with saturated sodium chloride, dried over sodium sulfate and concentrated. The crude product was purified on a silica gel column (40 g), using a gradient of 0 to 35% ethyl acetate in hexane. Pure fractions were combined and evaporated to give the title compound.

### Step 4: 5-Ethoxycarbonyl-4-methyl-3-phenylsulfonyl-1H-pyrrole-2-carboxylic acid

Ethyl 5-formyl-3-methyl-4-phenylsulfonyl-1H-pyrrole-2-carboxylate was dissolved in a mixture of t-butanol (25 mL), 2-methyl-2-butene (5 mL), and tetrahydrofuran (5 mL). Sodium chlorite (0.428 g, 4.72 mmol) and sodium dihydrogen phosphate (0.436 g, 3.63 mmol) were dissolved in water (5 mL) and this solution was added to the solution of ethyl 5-formyl-3-methyl-4-phenylsulfonyl-1H-pyrrole-2-carboxylate. The resulting mixture was stirred 1 hour at room temperature. Water was added (50 mL) and the reaction solution was extracted with ethyl acetate. The organic phase was set aside. The aqueous phase was acidified with 1M HCl until the pH was less than 2, and then it was extracted with ethyl acetate. This organic phase was washed with saturated brine, dried over sodium sulfate and concentrated to give the title compound.

### Step 5: Ethyl 5-aminocarbonyl-3-methyl-4-phenylsulfonyl-1H-pyrrole-2-carboxylate

5-Ethoxycarbonyl-4-methyl-3-phenylsulfonyl-1H-pyrrole-2-carboxylic acid (1.22 g, 3.63 mmol) was dissolved in dioxane. To this solution was added pyridine (0.177 mL, 2.18 mmol), di-t-butyl dicarbonate (1.03 g, 4.72 mmol) and ammonium carbonate (0.374 g, 4.72 mmol). The reaction was stirred at room temperature for three days. Ethyl acetate was added and the reaction mixture was washed with saturated sodium bicarbonate followed by saturated brine. The resulting organic phase was dried over sodium sulfate and concentrated to give the title compound.

### Step 6: 5-Aminocarbonyl-3-methyl-4-phenylsulfonyl-1H-pyrrole-2-carboxylic acid

Ethyl 5-aminocarbonyl-3-methyl-4-phenylsulfonyl-1H-pyrrole-2-carboxylate (0.550 g, 1.63 mmol) was dissolved in methanol (6 mL). A solution of lithium hydroxide hydrate (0.274 mL, 6.54 mmol) in water (5 mL) was added and the reaction stirred at 60° C. overnight. The methanol was removed with a stream of nitrogen, and the residue was suspended in 1 M HCl. The suspension was stirred for 15 minutes and filtered. The filter cake was washed with water and dried to give the title compound.

### Step 7: 5-Aminocarbonyl-3-methyl-4-phenylsulfonyl-1H-pyrrole-2-carbonyl chloride

A suspension of 5-aminocarbonyl-3-methyl-4-phenylsulfonyl-1H-pyrrole-2-carboxylic acid (0.110 g, 0.357 mmol) in thionyl chloride (2 mL) was refluxed for 1.5 hours. Excess thionyl chloride was removed under vacuum to give the title compound as an off white solid.

### Step 8: N-(2,4-Dichlorobenzyl)-N,3-dimethyl-4-(1-phenylsulfonyl)-1H-pyrrole-2,5-dicarboxamide

A solution of N-methyl 2,4-dichlorobenzylamine (0.015 g, 0.077 mmol) and triethylamine (0.011 mL, 0.084 mmol) in dry chloroform (1 mL) was cooled to 0° C. under nitrogen. To this solution was added a solution of 5-aminocarbonyl-3-methyl-4-phenylsulfonyl-1H-pyrrole-2-carbonyl chloride in chloroform (0.5 mL). The cooling bath was removed and the reaction stirred for 30 minutes at room temperature. The volatiles were removed in vacuo and the crude product purified by reverse phase HPLC (gradient 0.1% TFA/acetonitrile and 0.1% aq. TFA). Pure fractions were combined to obtain the title compound. MS (m+1) 480.0545

### Example 2

### N-(2,4-Dichlorobenzyl)-4-[(3,5-dichlorophenyl)sulfonyl]-N,3-dimethyl-1H-pyrrole-2,5-dicarboxamide

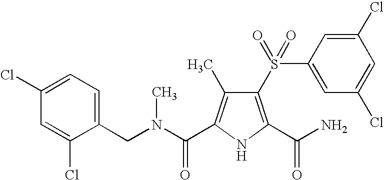

### Step 1: Ethyl 4-[(3,5-dichlorophenyl)thio]-3,5-dimethyl-1H-pyrrole-2-carboxylate

A solution of 3,5-dichlorobenzenethiol (2.14 g, 11.96 mmol) and triethylamine (5 drops) in dry dichloromethane (20 mL) was cooled to 0° C. To this mixture was added a solution of sulfuryl chloride (0.970 mL, 11.96 mmol) in dichloromethane. The reaction was stirred under nitrogen for 1 hour at room temperature. The dichloromethane was evaporated in vacuo, and the residue re-dissolved in dry dichloromethane (20 mL). This solution was added to a solution of ethyl 3,5-dimethyl-1H-pyrrole-2-carboxylate (1.00 g, 5.98 mmol) in dry dichloromethane (20 mL). The reaction was stirred for 1 hour at room temperature, and then quenched with saturated aq. sodium bicarbonate. After stirring overnight, the layers were separated and the organic phase washed with saturated brine and dried over sodium sulfate. The crude product was purified by chromatography on silica gel using gradient elution with 0-10% ethyl acetate in hexanes to give the title compound.

### Step 2: Ethyl 4-[(3,5-dichlorophenyl)sulfonyl]-3,5-dimethyl-1H-pyrrole-2-carboxylate

The title compound was prepared from ethyl 4-[(3,5-dichlorophenyl)thio]-3,5-dimethyl-1H-pyrrole-2-carboxylate using the procedure described in Example 1, Step 2.

### Step 3: Ethyl 4-[(3,5-dichlorophenyl)sulfonyl]-5-formyl-3-methyl-1H-pyrrole-2-carboxylate

Ethyl 4-[(3,5-dichlorophenyl)sulfonyl]-3,5-dimethyl-1H-pyrrole-2-carboxylate (0.870 g, 2.31 mmol) was dissolved in dichloromethane (30 mL) and a solution of sulfuryl chloride (0.609 mL, 7.51 mmol) in methylene chloride (4 mL) was added at 0° C. The resulting mixture was stirred at room temperature for 2 hours, then slowly added to a solution of refluxing aq. acetone (acetone:water 2:1, 60 mL). The reaction was refluxed for 15 minutes, and then cooled to room temperature. Acetone was evaporated under reduced pressure and the product was extracted with dichloromethane. The crude product was purified by silica gel chromatography (120 g silica gel, 0 to 35% ethyl acetate in hexane gradient) and pure fractions combined to give the title compound.

### Step 4: N-(2,4-Dichlorobenzyl)-4-[(3,5-dichlorophenyl)sulfonyl]-N,3-dimethyl-1H-pyrrole-2,5-dicarboxamide

The title compound was prepared using the procedures described for Example 1, Step 4-Step 8, substituting ethyl 4-[(3,5-dichlorophenyl)sulfonyl]-5-formyl-3-methyl-1H-pyrrole-2-carboxylate in place of ethyl 5-formyl-3-methyl-4-phenylsulfonyl-1H-pyrrole-2-carboxylate, proceeding through analogous intermediates. MS (M+1) 549.9724

### Example 3

### N-Benzyl-N,3-dimethyl-4-(3,5-dimethylphenylsulfonyl)-1H-pyrrole-2,5-dicarboxamide

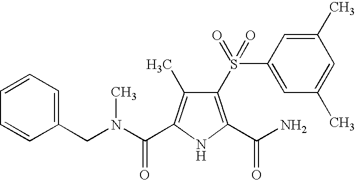

### Step 1: Ethyl 4-[(3,5-dimethylphenyl)thio]-3,5-dimethyl-1H-pyrrole-2-carboxylate

The title compound was prepared from ethyl 3,5-dimethyl-1H-pyrrole-2-carboxylate according to the procedure described in Example 2, Step 1, except using 3,5-dimethylthiophenol in place of 3,5-dichlorothiophenol.

### Step 2: N-benzyl-N,3-dimethyl-4-(3,5-dimethylphenylsulfonyl)-1H-pyrrole-2,5-dicarboxamide

The title compound was prepared according to the procedure described in Example 2 Steps 2-4, except using ethyl 4-[(3,5-dimethylphenyl)thio]-3,5-dimethyl-1H-pyrrole-2-carboxylate in place of ethyl 4-[(3,5-dichlorophenyl)thio]-3,5-dimethyl-1H-pyrrole-2-carboxylate, and N-methylbenzylamine in place of N-methyl 2,4-dichlorobenzylamine, and proceeding through analogous intermediates. MS (M+1) 440.1648

### Example 4

### N-(2-Chloro-4-fluorobenzyl)-N,3-dimethyl-4-(phenylsulfonyl)-1H-pyrrole-2,5-dicarboxamide

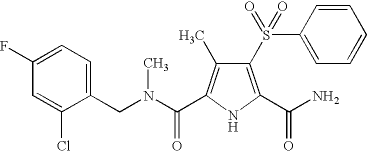

### Step 1: N-Methyl 2-chloro-4-fluorobenzylamine

2-Chloro-4-fluorobenzaldehyde (0.500 g, 3.15 mmol) was dissolved in methanol (10 mL) and a solution of methylamine in methanol (2N, 3.15 mL, 6.30 mmol) was added. The resulting mixture was sealed in a tube and heated at 60° C. for 2 hours. The solvent and excess methylamine were removed in vacuo and the residue dissolved in ethanol (10 mL) and cooled to 0° C. Sodium borohydride (0.155 g, 4.09 mmol) was added in portions. The resulting mixture was stirred 1 hour at room temperature. Ethanol was removed in vacuo, and the residue dissolved in dichloromethane (15 mL). This solution was washed with 2% sodium hydroxide solution, saturated sodium bicarbonate and saturated brine, and then dried over sodium sulfate and concentrated to give the title compound.

### Step 2: N-(2-Chloro-4-fluorobenzyl)-N,3-dimethyl-4-(phenylsulfonyl)-1H-pyrrole-2,5-dicarboxamide

The title compound was obtained from 5-aminocarbonyl-3-methyl-4-phenylsulfonyl-1H-pyrrole-2-carbonyl chloride according to the procedure described in Example 1, Step 8, except using N-methyl 2-chloro-4-fluorobenzylamine in place of N-methyl 2,4-dichlorobenzylamine, to give the title compound. MS (M+1) 464.0844

### Example 5

### N-(2-Chlorobenzyl)-N,3-dimethyl-4-(1-naphthylsulfonyl)-1H-pyrrole-2,5-dicarboxamide

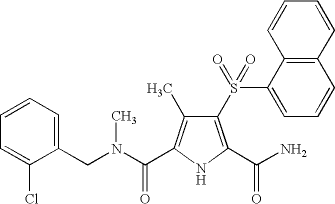

### Step 1: Ethyl 3,5-dimethyl-4-(1-naphthylsulfonyl)-1H-pyrrole-2-carboxylate and ethyl 3,5-dimethyl-4-(2-naphthylsulfonyl)-1H-pyrrole-2-carboxylate

Ethyl 4-(chlorosulfonyl)-3,5-dimethyl-1H-pyrrole-2-carboxylate (0.777 g, 2.92 mmol) (prepared according to the procedure described in Moranta, C. et al., J. Chem. Soc., Perkin Trans. 1, 1998 (19) 3285), naphthalene (0.375 g, 2.92 mmol), and indium (III) chloride (0.129 g, 0.585 mmol) were stirred in trifluoroacetic acid (6 mL). Trifluoromethysulfonic acid (0.388 mL, 4.39 mmol) was added to the solution and the resulting reaction was stirred for 15 minutes at room temperature. The reaction was quenched via dropwise addition into ice water and a precipitate formed. The solid was filtered, washed with water and taken up in dichloromethane. This was washed with saturated sodium bicarbonate, water, saturated brine, dried with sodium sulfate, filtered and concentrated in vacuo. The residue was purified via flash chromatography on silica gel column (254 mm×40 mm) with 20% to 30% ethyl acetate/hexane gradient elution to separate the title compounds.

### Step 2: N-(2-Chlorobenzyl)-N,3-dimethyl-4-(1-naphthylsulfonyl)-1H-pyrrole-2,5-dicarboxamide

The title compound was obtained from ethyl 3,5-dimethyl-4-(1-naphthylsulfonyl)-1H-pyrrole-2-carboxylate using procedures similar to those described in Example 2 and using the appropriate starting materials. MS (M+1) 496.1073.

### Example 6

### N-(2-Chlorobenzyl)-N,3-dimethyl-4-(2-naphthylsulfonyl)-1H-pyrrole-2,5-dicarboxamide

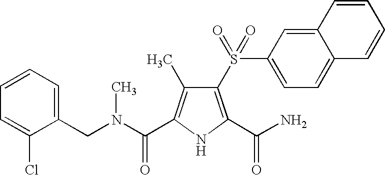

The title compound was prepared from ethyl 3,5-dimethyl-4-(2-naphthylsulfonyl)-1H-pyrrole-2-carboxylate (see Example 5, Step 1) using a procedure similar that in Example 2. MS (M+1) 496.1071

### Examples 7-37

The compounds in Table A below were prepared using a procedure similar to that employed in Examples 1-6. The table provides the structure and name of each compound and the mass of its molecular ion plus 1 (M+1) as determined via MS.

TABLE A

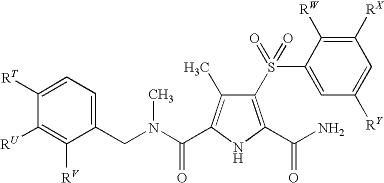

| Ex. | Compound | R^{T} | R^{U} | R^{V} | R^{W} | R^{X} | R^{Y} | M + 1 |
|---|---|---|---|---|---|---|---|---|
| 7 | N-(2-fluorobenzyl)-N,3- dimethyl-4-(phenylsulfonyl)- 1H-pyrrole-2,5-dicarboxamide | H | H | F | H | H | H | 430.1228 |
| 8 | N-(2-chlorobenzyl)-N,3- dimethyl-4-(phenylsulfonyl)- 1H-pyrrole-2,5-dicarboxamide | H | H | Cl | H | H | H | 446.0926 |
| 9 | N-(2-bromobenzyl)-N,3- dimethyl-4-(phenylsulfonyl)- 1H-pyrrole-2,5-dicarboxamide | H | H | Br | H | H | H | 492.0404 |
| 10 | N-(3-chlorobenzyl)-N,3- dimethyl-4-(phenylsulfonyl)- 1H-pyrrole-2,5-dicarboxamide | H | Cl | H | H | H | H | 446.0928 |
| 11 | N-(4-bromobenzyl)-N,3- dimethyl-4-(phenylsulfonyl)- 1H-pyrrole-2,5-dicarboxamide | Br | H | H | H | H | H | 490.0450 |
| 12 | N-(4-chloro-2-fluorobenzyl)- N,3-dimethyl-4- (phenylsulfonyl)-1H-pyrrole- 2,5-dicarboxamide | Cl | H | F | H | H | H | 525.2285 |
| 13 | N-(2,3-dichlorobenzyl)-N,3- dimethyl-4-(phenylsulfonyl)- 1H-pyrrole-2,5-dicarboxamide | H | Cl | Cl | H | H | H | 480.0548 |
| 14 | N-(3,4-dichlorobenzyl)-N,3- dimethyl-4-(phenylsulfonyl)- 1H-pyrrole-2,5-dicarboxamide | Cl | Cl | H | H | H | H | 480.0542 |
| 15 | N-(2-chloro-4- methylsulfonylbenzyl)-N,3- dimethyl-4-(phenylsulfonyl)- 1H-pyrrole-2,5-dicarboxamide | SO₂CH₃ | H | Cl | H | H | H | 524.0697 |
| 16 | N-(2-fluorobenzyl)-N,3- dimethyl-4-(3- fluorophenylsulfonyl)-1H- pyrrole-2,5-dicarboxamide | H | H | F | H | F | H | 448.1161 |
| 17 | N-(2-chlorobenzyl)-N,3- dimethyl-4-(3- fluorophenylsulfonyl)-1H- pyrrole-2,5-dicarboxamide | H | H | Cl | H | F | H | 464.0839 |
| 18 | N-(4-chloro-2-fluorobenzyl)- N,3-dimethyl-4-(3- fluorophenylsulfonyl)-1H- pyrrole-2,5-dicarboxamide | Cl | H | F | H | F | H | 482.0761 |
| 19 | N-(2,4-dichlorobenzyl)-N,3- dimethyl-4-(3- fluorophenylsulfonyl)-1H- pyrrole-2,5-dicarboxamide | Cl | H | Cl | H | F | H | 498.0449 |
| 20 | N-(2-chlorobenzyl)-N,3- dimethyl-4-(3- chlorophenylsulfonyl)-1H- pyrrole-2,5-dicarboxamide | H | H | Cl | H | Cl | H | 480.0542 |
| 21 | N-(2,4-dichlorobenzyl)-N,3- dimethyl-4-(3- chlorophenylsulfonyl)-1H- pyrrole-2,5-dicarboxamide | Cl | H | Cl | H | Cl | H | 516.0115 |
| 22 | N-benzyl-N,3-dimethyl-4-(3- trifluoromethylphenylsulfonyl)- 1H-pyrrole-2,5-dicarboxamide | H | H | H | H | CF₃ | H | 480.1220 |
| 23 | N-(2,4-dichlorobenzyl)-N,3- dimethyl-4-(3- trifluoromethylphenylsulfonyl)- 1H-pyrrole-2,5-dicarboxamide | Cl | H | Cl | H | CF₃ | H | 548.0405 |
| 24 | N-(2-fluorobenzyl)-N,3- dimethyl-4-(3,5- dimethylphenylsulfonyl)-1H- pyrrole-2,5-dicarboxamide | H | H | F | H | CH₃ | CH₃ | 458.1545 |
| 25 | N-(2-chlorobenzyl)-N,3- dimethyl-4-(3,5- dimethyiphenylsulfonyl)-1H- pyrrole-2,5-dicarboxamide | H | H | Cl | H | CH₃ | CH₃ | 474.1254 |
| 26 | N-(4-chloro-2-fluorobenzyl)- N,3-dimethyl-4-(3,5- dimethylphenylsulfonyl)-1H- pyrrole-2,5-dicarboxamide | Cl | H | F | H | CH₃ | CH₃ | 492.1155 |
| 27 | N-(2,4-dichlorobenzyl)-N,3- dimethyl-4-(3,5- dimethylphenylsulfonyl)-1H- pyrrole-2,5-dicarboxamide | Cl | H | Cl | H | CH₃ | CH₃ | 508.0862 |
| 28 | N-benzyl-N,3-dimethyl-4-(3- chloro-5- fluorophenylsulfonyl)-1H- pyrrole-2,5-dicarboxamide | H | H | H | H | Cl | F | 464.0858 |
| 29 | N-(2-fluorobenzyl)-N,3- dimethyl-4-(3-chloro-5- fluorophenylsulfonyl)-1H- pyrrole-2,5-dicarboxamide | H | H | F | H | Cl | F | 482.0754 |
| 30 | N-(2-chlorobenzyl)-N,3- dimethyl-4-(3-chloro-5- fluorophenylsulfonyl)-1H- pyrrole-2,5-dicarboxamide | H | H | Cl | H | Cl | F | 498.0445 |
| 31 | N-(4-chloro-2-fluorobenzyl)- N,3-dimethyl-4-(3-chloro-5- fluorophenylsulfonyl)-1H- pyrrole-2,5-dicarboxamide | Cl | H | F | H | Cl | F | 516.0349 |
| 32 | N-benzyl-N,3-dimethyl-4-(3,5- dichlorophenylsulfonyl)-1H- pyrrole-2,5-dicarboxamide | H | H | H | H | Cl | Cl | 480.0545 |
| 33 | N-(2-fluorobenzyl)-N,3- dimethyl-4-(3,5- dichlorophenylsulfonyl)-1H- pyrrole-2,5-dicarboxamide | H | H | F | H | Cl | Cl | 498.0426 |
| 34 | N-(2-chlorobenzyl)-N,3- dimethyl-4-(3,5- dichlorophenylsulfonyl)-1H- pyrrole-2,5-dicarboxamide | H | H | Cl | H | Cl | Cl | 514.0152 |
| 35 | N-(4-chloro-2-fluorobenzyl)- N,3-dimethyl-4-(3,5- dichlorophenylsulfonyl)-1H- pyrrole-2,5-dicarboxamide | Cl | H | F | H | Cl | Cl | 532.0038 |
| 36 | N-(3-methoxyobenzyl)-N,3- dimethyl-4-(phenylsulfonyl)- 1H-pyrrole-2,5-dicarboxamide | H | OCH₃ | H | H | H | H | 442.1423 |
| 37 | N-(2-chlorobenzyl)-N,3- dimethyl-4-(2-cyano-3- methyiphenylsulfonyl)-1H- pyrrole-2,5 -dicarboxamide | H | H | Cl | CN | CH₃ | H | 501.1015 |

### Examples 38-39

The compounds in Table B below were prepared using a procedure similar to that employed in Example 4. The table provides the structure and name of each compound and the mass of its molecular ion plus 1 (M+1) as determined via MS. When the compound was prepared as a salt, the identity of the salt is included in parentheses following the compound name for the free base.

TABLE B

| Ex. | Compound | Structure | M + 1 |
|---|---|---|---|
| 38 | N,3-dimethyl-4- (phenylsulfonyl)-N-(3- thienylmethyl)-1H-pyrrole-2,5- dicarboxamide | 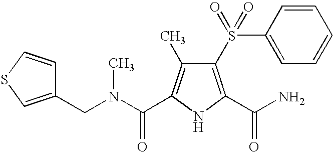 | 418.0894 |
| 39 | N-[(3-chloro-4- pyridinyl)methyl]-N,3-dimethyl- 4-(phenylsulfonyl)-1H-pyrrole- 2,5-dicarboxamide (trifluoracetic acid salt) | 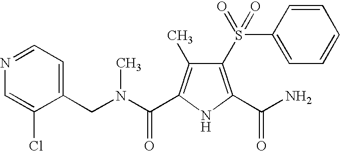 | 447.0876 |

### Example 40

### 4-[(3,5-Dimethylphenyl)sulfonyl]-N,3-dimethyl-N-(6-quinolinylmethyl)-1H-pyrrole-2,5-dicarboxamide

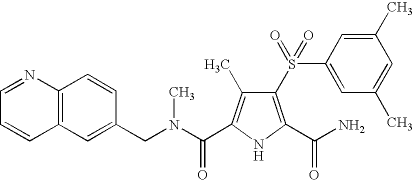

A solution of 5-aminocarbonyl-4-[(3,5-dimethylphenyl)sulfonyl]-3-methyl-1H-pyrrole-2-carboxylic acid (0.025 g, 0.074 mmol), N-hydroxybenzotriazole (0.015 g, 0.097 mmol), N-(3-dimethylaminopropyl) N′-ethylcarbodiimide hydrochloride (0.019 g, 0.097 mmol) and N-methyl-1-(6-quinolinyl)methanamine (0.013 g, 0.074 mmol) in dimethylformamide (1 mL) was stirred for 3 hours. The product was isolated by reverse phase HPLC using gradient elution with 0.1% trifluoroacetic acid in acetonitrile and 0.1% trifluoroacetic acid in water. The pure fractions were collected, concentrated and dissolved in ethyl acetate. The ethyl acetate solution was washed with saturated sodium bicarbonate and saturated brine, dried over sodium sulfate and concentrated. The pure compound was dissolved in methanolic hydrochloric acid, then diluted with water. The resulting suspension was lyophilized to give the title compound as the hydrochloride salt. MS (M+1) 491.1761

### Example 41

### N-[(7-Chloro-6-quinolinyl)methyl]-N,3-dimethyl-4-(phenylsulfonyl)-1H-pyrrole-2,5-dicarboxamide

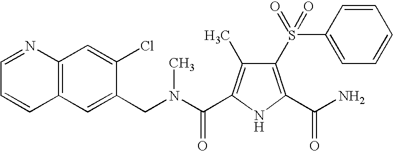

### Step 1: 6-(Bromomethyl)-7-chloroquinoline

A solution of 7-chloro-6-methylquinoline (3.50 g, 19.70 mmol) (prepared according to the procedure described in Corn et al., J. Am. Chem. Soc. 1930, 52, 3685), benzoyl peroxide (0.048 g, 0.197 mmol), and N-bromosuccinimide (7.01 g, 39.40 mmol) in carbon tetrachloride (200 mL) was refluxed overnight. The reaction mixture was loaded onto a silica gel chromatography column (120 g column) and eluted with 0 to 25% ethyl acetate in hexane. The pure product fractions were combined and solvent removed to give the title compound.

### Step 2: 1-(7-Chloro-6-quinolinyl)-N-methylmethanamine

A solution of 6-(bromomethyl)-7-chloroquinoline (0.050 g, 0.195 mmol) in methanol (0.5 mL) was added to a solution of 2N methylamine in methanol (1 mL). The reaction mixture was heated in a sealed tube at 65° C. for 1 hour. The crude product was purified by silica gel chromatography (4 g silica gel column) using 2 to 10% methanol/dichloromethane gradient elution to obtain the title compound.

### Step 3: N-[(7-Chloro-6-quinolinyl)methyl]-N,3-dimethyl-4-(phenylsulfonyl)-1H-pyrrole-2,5-dicarboxamide

The title compound was obtained according to the procedure described in Example 1, Step 8, except using 1-(7-chloro-6-quinolinyl)-N-methylmethanamine in place of N-methyl 2,4-dichlorobenzylamine. After purification, the title compound was obtained as the trifluoroacetate salt. MS (M+1) 497.1024

### Example 42

### N-[(5-Chloro-6-quinolinyl)methyl]-N,3-dimethyl-4-(phenylsulfonyl)-1H-pyrrole-2,5-dicarboxamide

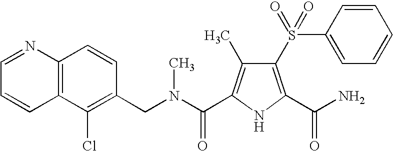

The title compound was prepared according to the procedure described in Steps 1-3 of Example 41, except 5-chloro-6-methylquinoline (Corn et al., J. Am. Chem. Soc. 1930, 52, 3685) was employed in place of 7-chloro-6-methylquinoline. After purification, the title compound was obtained as the trifluoroacetate salt. MS (M+1) 497.1032

### Examples 43-57

The compounds in Table C below were prepared using a procedure similar to that employed in Example 40. The table provides the structure and name of each compound and the mass of its molecular ion plus 1 (M+1) as determined via MS. When the compound was prepared as a salt, the identity of the salt is included in parentheses following the compound name for the free base.

TABLE C

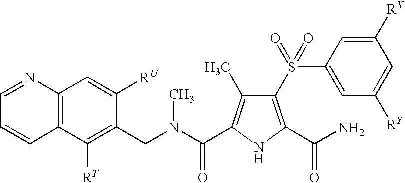

| Ex. | Compound | R^{T} | R^{U} | R^{X} | R^{Y} | M + 1 |
|---|---|---|---|---|---|---|
| 43 | N,3-dimethyl-4-(phenylsulfonyl- N-(6-quinolinylmethyl)-1H- pyrrole-2,5-dicarboxamide (trifluoroacetic acid salt) | H | H | H | H | 463.1448 |
| 44 | N,3-dimethyl-4-(3- methylphenylsulfonyl-N-(6- quinolinylmethyl)-1H-pyrrole-2,5- dicarboxamide (trifluoroacetic acid salt) | H | H | CH₃ | H | 477.1598 |
| 45 | 4-(3-fluorophenylsulfonyl-N,3- dimethyl-N-(6-quinolinylmethyl)- 1H-pyrrole-2,5-dicarboxamide (trifluoroacetic acid salt) | H | H | F | H | 481.1325 |
| 46 | N,3-dimethyl-N-(6- quinolinylmethyl)-4-{[3- (trifluoromethyl)phenyl]sulfonyl}- 1H-pyrrole-2,5-dicarboxamide (trifluoroacetic acid salt) | H | H | CF₃ | H | 531.1314 |
| 47 | N-[(5-chloro-6- quinolinyl)methyl]-4-[(3,5- dimethylphenyl)sulfonyl]-N,3- dimethyl-1H-pyrrole-2,5- dicarboxamide (trifluoroacetic acid salt) | Cl | H | CH₃ | CH₃ | 525.1329 |
| 48 | N-[(7-chloro-6- quinolinyl)methyl]-4-[(3,5- dimethylphenyl)sulfonyl]-N,3- dimethyl-1H-pyrrole-2,5- dicarboxamide (trifluoroacetic acid salt) | H | Cl | CH₃ | CH₃ | 525.1340 |
| 49 | N-[(5-chloro-6- quinolinyl)methyl]-4-[(3,5- difluorophenyl)sulfonyl]-N,3- dimethyl-1H-pyrrole-2,5- dicarboxamide (trifluoroacetic acid salt) | Cl | H | F | F | 533.0864 |
| 50 | N-[(7-chloro-6- quinolinyl)methyl]-4-[(3,5- difluorophenyl)sulfonyl]-N,3- dimethyl-1H-pyrrole-2,5- dicarboxamide (trifluoroacetic acid salt | H | Cl | F | F | 533.0885 |
| 51 | 4-[(3-chloro-5- fluorophenyl)sulfonyl]-N,3- dimethyl-N-(6-quinolinylmethyl)- 1H-pyrrole-2,5-dicarboxamide (trifluoroacetic acid salt) | H | H | Cl | F | 515.0949 |
| 52 | 4-[(3-chloro-5- fluorophenyl)sulfonyl]-N-[(5- chloro-6-quinolinyl)methyl]-N,3- dimethyl-1H-pyrrole-2,5- dicarboxamide (trifluoroacetic acid salt) | Cl | H | Cl | F | 549.0564 |
| 53 | 4-[(3-chloro-5- fluorophenyl)sulfonyl]-N-[(7- chloro-6-quinolinyl)methyl]-N,3- dimethyl-1H-pyrrole-2,5- dicarboxamide (trifluoroacetic acid salt) | H | Cl | Cl | F | 549.0557 |
| 54 | 4-[(3,5-dichlorophenyl)sulfonyl]- N,3-dimethyl-N-(6- quinolinylmethyl)-1H-pyrrole-2,5- dicarboxamide (trifluoroacetic acid salt) | H | H | Cl | Cl | 531.0666 |
| 55 | N-[(5-chloro-6- quinolinyl)methyl]-4-[(3,5- dichlorophenyl)sulfonyl]-N,3- dimethyl-1H-pyrrole-2,5- dicarboxamide (trifluoroacetic acid salt) | Cl | H | Cl | Cl | 565.0265 |
| 56 | N-[(7-chloro-6- quinolinyl)methyl]-4-[(3,5- dichlorophenyl)sulfonyl]-N,3- dimethyl-1H-pyrrole-2,5- dicarboxamide (trifluoroacetic acid salt) | H | Cl | Cl | Cl | 567.0227 |
| 57 | 4-[(3-chloro-5- cyanophenyl)sulfonyl]-N,3- dimethyl-N-[(6-quinolinylmethyl]- 1H-pyrrole-2,5-dicarboxamide (trifluoroacetic acid salt) | H | H | Cl | CN | 522.0970 |

### Example 58

### N-Benzyl-3-isopropyl-N-methyl-4-(phenylsulfonyl)-1H-pyrrole-2,5-dicarboxamide

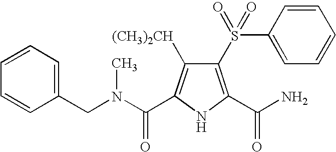

### Step 1: 5-(1-Hydroxy-2-methylpropylidene)-2,2-dimethyl-1,3-dioxane-4,6-dione

Meldrum's acid (20.0 g, 138.7 mmol) was dissolved in dry dichloromethane (250 mL) and the solution cooled to 0° C. with an ice-salt bath. Pyridine (22.4 mL, 277.5 mmol) was added, followed by the dropwise addition of isobutyryl chloride (21.95 g, 277.5 mmol), keeping the temperature below 10° C. The resulting mixture was stirred at 0° C. for 30 minutes and at room temperature for 3 hours. The reaction mixture was washed with 7.5% aq. hydrochloric acid (˜320 mL), saturated brine and dried over sodium sulfate. The solvent was evaporated in vacuo to give the title compound.

### Step 2: tert-Butyl 4-methyl-3-oxopentanoate

5-(1-Hydroxy-2-methylpropylidene)-2,2-dimethyl-1,3-dioxane-4,6-dione (21.00 g, 98.03 mmol) and t-butanol (27.6 mL, 294.1 mmol) was dissolved in benzene (200 mL) and the reaction mixture was refluxed overnight. The benzene was evaporated in vacuo to give the title compound.

Step 3 4-Benzyl 2-tert-butyl 3-isopropyl-5-methyl-1H-pyrrole-2,4-dicarboxylate

A solution of sodium nitrite (6.33 g, 91.8 mmol) in water (24 mL) was added to a solution of tert-butyl 4-methyl-3-oxopentanoate (18.00 g, 96.64 mmol) in acetic acid (90 mL), and the reaction was stirred overnight. This reaction mixture was added to a mixture of acetoacetic acid benzyl ester (18.37 mL, 106.3 mmol), ammonium acetate (18.62 g, 241.6 mmol) and zinc metal (18.95 g, 289.9 mmol) in acetic acid (60 mL), where the rate of addition was adjusted to maintain the internal temperature around 55° C. After 6 hours, the reaction mixture was poured into ice water (500 mL) and dichloromethane (500 mL), stirred for 10 minutes and filtered; the filtered solid was rinsed with dichloromethane. The filtrate was separated into organic and aqueous phases, and the aqueous phase was back extracted with dichloromethane (200 mL×2). The combined organic phases were washed with saturated brine, saturated brine/sodium bicarbonate, and then dried over sodium sulfate and concentrated in vacuo. The crude product was dissolved in 1:2 ethyl acetate/hexane (450 mL), and let stand for 72 hours. An oily residue separated and was discarded. The remaining solution was filtered through a silica gel pad (50 g), and washed with 1:2 ethyl acetate/hexane (100 mL×2). The filtrate was concentrated in vacuo and the crude product was chromatographed on silica gel using gradient elution with 0-10% ethyl acetate in hexanes to give the title compound.

### Step 4 5-(tert-Butoxycarbonyl)-4-isopropyl-2-methyl-1H-pyrrole-3-carboxylic acid

A solution of 4-benzyl 2-tert-butyl 3-isopropyl-5-methyl-1H-pyrrole-2,4-dicarboxylate (5.00 g, 13.9 mmol) in methanol (100 mL) was purged with nitrogen and 10% Pd/C (150 mg) was added. A hydrogen atmosphere was established (1 atm) and the reaction stirred 3 hours. The catalyst was filtered and the filtrate concentrated in vacuo to give the title compound.

### Step 5: tert-Butyl 4-iodo-3-isopropyl-5-methyl-1H-pyrrole-2-carboxylate

A solution of iodine (3.20 g, 12.64 mmol) and sodium iodide (3.79 g, 25.28 mmol) in water (25 mL) was added to 5-(tert-butoxycarbonyl)-4-isopropyl-2-methyl-1H-pyrrole-3-carboxylic acid (2.60 g, 9.72 mmol) and sodium bicarbonate (2.69 g, 32.09 mmol) in 1:1 dichloroethane/water (50 mL). The reaction mixture was stirred at 100° C. for 40 minutes. The reaction was cooled to room temperature and diluted with dichloromethane (100 mL). The reaction was washed with aq. sodium bicarbonate solution. The crude product was purified by silica gel chromatography using 0 to 15% ethyl acetate/hexane gradient elution. Pure fractions were combined and concentrated to give the title compound.

### Step 6: tert-Butyl 3-isopropyl-5-methyl-1H-pyrrole-2-carboxylate

tert-Butyl 4-iodo-3-isopropyl-5-methyl-1H-pyrrole-2-carboxylate (3.30 g, 9.45 mmol) was dissolved in methanol (150 mL) containing triethylamine (2 mL) and the solution was purged with nitrogen. 10% Pd/C (100 mg) was added and the mixture shaken overnight under 45 psi hydrogen on a Parr apparatus. The catalyst was filtered and the crude product purified by silica gel chromatography (120 g silica) using 0 to 25% ethyl acetate/hexane gradient elution.

### Step 7: tert-Butyl 5-(aminocarbonyl)-3-isopropyl-4-(phenylsulfonyl)-1H-pyrrole-2-carboxylate

The title compound was obtained starting with tert-butyl 3-isopropyl-5-methyl-1H-pyrrole-2-carboxylate and using the procedures described in Example 2, Steps 1-3, and Example 1, Steps 4-5.

### Step 8: 5-(Aminocarbonyl)-3-isopropyl-4-(phenylsulfonyl)-1H-pyrrole-2-carboxylic acid

tert-Butyl 5-(aminocarbonyl)-3-isopropyl-4-(phenylsulfonyl)-1H-pyrrole-2-carboxylate (0.116 g, 0.296 mmol) was stirred in dichloromethane (3 mL) with TFA (0.6 mL) at room temperature for 3 hours. Evaporation of solvent and TFA provided the title compound.

### Step 9: N-Benzyl-3-isopropyl-N-methyl-4-(phenylsulfonyl)-1H-pyrrole-2,5-dicarboxamide

The title compound was obtained using the procedures described in Example 40, except 5-(aminocarbonyl)-3-isopropyl-4-(phenylsulfonyl)-1H-pyrrole-2-carboxylic acid and N-benzyl-N-methylamine were employed. MS (M+1) 440.1624

### Examples 59-70

The compounds in Table D below were prepared using a procedure similar to that employed in Example 58. The table provides the structure and name of each compound and the mass of its molecular ion plus 1 (M+1) as determined via MS. When the compound was prepared as a salt, the identity of the salt is included in parentheses following the compound name for the free base.

TABLE D

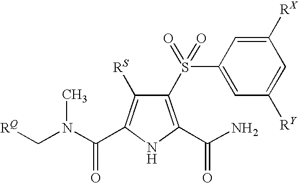

| Ex. | Compound | R^{Q} | R^{S} | R^{X} | R^{Y} | M + 1 |
|---|---|---|---|---|---|---|
| 59 | N-(2,4-dichlorobenzyl)-3-ethyl-N- methyl-4-(phenylsulfonyl)-1H- pyrrole-2,5-dicarboxamide | 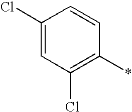 | Et | H | H | 494.0680 |
| 60 | 3-ethyl-N-methyl-4- (phenylsulfonyl)-N-(6- quinolinylmethyl)-1H-pyrrole-2,5- dicarboxamide (trifluoroacetic acid salt) | 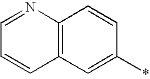 | Et | H | H | 477.1571 |
| 61 | N-(2-fluorobenzyl)-3-isopropyl-N- methyl-4-(phenylsulfonyl)-1H- pyrrole-2,5-dicarboxamide | 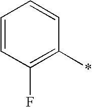 | i-Pr | H | H | 458.1460 |
| 62 | N-(4-chloro-2-fluorobenzyl)-3- isopropyl-N-methyl-4- (phenylsulfonyl)-1H-pyrrole-2,5- dicarboxamide | 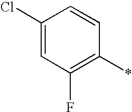 | i-Pr | H | H | 492.1067 |
| 63 | N-(2,4-dichlorobenzyl)-3- isopropyl-N-methyl-4- (phenylsulfonyl)-1H-pyrrole-2,5- dicarboxamide | 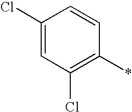 | i-Pr | H | H | 508.0827 |
| 64 | 3-isopropyl-N-methyl-4- (phenylsulfonyl)-N-(6- quinolinylmethyl)-1H-pyrrole-2,5- dicarboxamide (trifluoroacetic acid salt) | 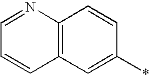 | i-Pr | H | H | 491.1722 |
| 65 | N-methyl-4-(phenylsulfonyl)-3- propyl-N-(6-quinolinylmethyl)- 1H-pyrrole-2,5-dicarboxamide (trifluoroacetic acid salt) | 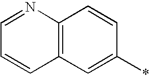 | i-Pr | H | H | 491.1720 |
| 66 | 3-butyl-N-methyl-4- (phenylsulfonyl)-N-(6- quinolinylmethyl)-1H-pyrrole-2,5- dicarboxamide (trifluoroacetic acid salt) | 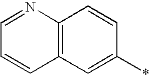 | n-Bu | H | H | 502.1881 |
| 67 | 3-butyl-N-methyl-4-(3- methylphenylsulfonyl)-N-(6- quinolinylmethyl)-1H-pyrrole-2,5- dicarboxamide (trifluoroacetic acid salt) | 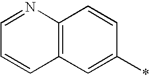 | n-Bu | CH₃ | H | 519.2034 |
| 68 | 3-butyl-4-[(3- chlorophenyl)sulfonyl]-N-methyl- N-(6-quinolinylmethyl)-1H- pyrrole-2,5-dicarboxamide (trifluoroacetic acid salt) | 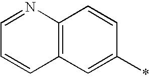 | n-Bu | Cl | H | 539.1493 |
| 69 | 3-butyl-4-[(3,5- dimethylphenyl)sulfonyl]-N- methyl-N-(6-quinolinylmethyl)- 1H-pyrrole-2,5-dicarboxamide (trifluoroacetic acid salt) | 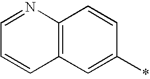 | n-Bu | CH₃ | CH₃ | 533.2188 |
| 70 | 3-butyl-4-[(3,5- dichlorophenyl)sulfonyl]-N- methyl-N-(6-quinolinylmethyl)- 1H-pyrrole-2,5-dicarboxamide (trifluoroacetic acid salt) | 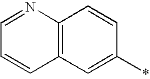 | n-Bu | Cl | Cl | 573.1110 |
| 70A | 3-isopropyl-N-methyl-4- (phenylsulfonyl)-N-[(5- chloroquinolin-6-yl)methyl]-1H- pyrrole-2,5-dicarboxamide (trifluoroacetic acid salt) | 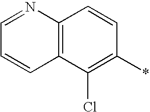 | i-Pr | H | H | 525.1378 |
| 70B | 3-isopropyl-N-methyl-4- (phenylsulfonyl)-N-[(7- chloroquinolin-6-yl)methyl]-1H- pyrrole-2,5-dicarboxamide (trifluoroacetic acid salt) | 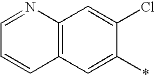 | i-Pr | H | H | 525.1385 |

### Example 71

### N-(2,4-Dichlorobenzyl)-N′-(1H-indazol-3-ylmethyl)-N,3-dimethyl-4-(phenylsulfonyl)-1H-pyrrole-2,5-dicarboxamide

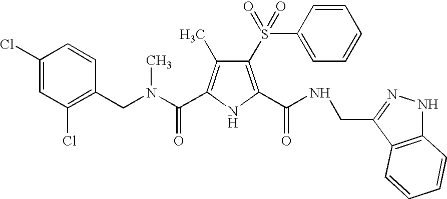

### Step 1: 3,5-Dimethyl-4-(phenylsulfonyl)-1H-pyrrole-2-carboxylic acid

Ethyl 3,5-dimethyl-4-phenylsulfonyl-1H-pyrrole-2-carboxylate (3.00 g, 9.76 mmol) was dissolved in methanol (300 mL) and water (5 mL). Lithium hydroxide hydrate (2.04 g, 48.80 mmol) was added and the reaction refluxed overnight. The methanol was removed in vacuo and the crude product was dissolved in water (20 mL) and acidified with 12N HCl. After stirring for 30 minutes, the solid was filtered, washed with water and dried to give the title compound.

### Step 2: 3,5-Dimethyl-4-(phenylsulfonyl)-1H-pyrrole-2-carbonyl chloride

3,5-Dimethyl-4-(phenylsulfonyl)-1H-pyrrole-2-carboxylic acid (1.00 g, 3.58 mmol) was refluxed for 1.5 hours in thionyl chloride (10 mL). The excess thionyl chloride was removed in vacuo to give the title compound as a white solid.

### Step 3: N-2,4-(Dichlorobenzyl)-N,3,5-trimethyl-4-(phenylsulfonyl)-1H-pyrrole-2-carboxamide

A solution of N-methyl 2,4-dichlorobenzylamine (0.702 g, 3.69 mmol) and triethylamine (0.504 mL, 3.69 mmol) in dry chloroform (10 mL) was cooled to 0° C. 3,5-Dimethyl-4-(phenylsulfonyl)-1H-pyrrole-2-carbonyl chloride (1.00 g, 3.35 mmol) in dry chloroform (20 mL) was added to the solution of the amine. The cooling bath was removed and stirring continued for 30 minutes at room temperature.

The reaction mixture was directly loaded to a silica gel column (40 g silica gel) and the product purified using 0 to 35% ethyl acetate/dichloromethane gradient elution. The pure fractions were combined to give the title compound.

### Step 4: N-(2,4-Dichlorobenzyl)-5-formyl-N,3-dimethyl-4-(phenylsulfonyl)-1H-pyrrole-2-carboxamide

N-2,4-(Dichlorobenzyl)-N,3,5-trimethyl-4-(phenylsulfonyl)-1H-pyrrole-2-carboxamide (1.30 g, 2.88 mmol) was dissolved in dichloromethane (20 mL) and cooled to 0° C. A solution of sulfuryl chloride (0.759 mL, 9.36 mmol) in dichloromethane (6 mL) was added dropwise with stirring. The reaction mixture was stirred at room temperature 2 hours, then added slowly to boiling aqueous acetone (2:1 acetone:water, 75 mL). The reaction was kept at this temperature for 15 minutes, then cooled to room temperature. Acetone was evaporated under reduced pressure and the product was extracted with dichloromethane. The organic phase was washed with saturated brine and dried over sodium sulfate. The crude product was purified on a silica gel column (40 g silica gel) using 0 to 40% ethyl acetate gradient elution. Pure fractions were combined to give the title compound.

### Step 5: 5-{[(2,4-Dichlorobenzyl)(methyl)amino]carbonyl}-4-methyl-3-(phenylsulfonyl)-1H-pyrrole-2-carboxylic acid

Sodium chlorite (0.111 g, 1.22 mmol) and sodium dihydrogen phosphate (0.113 g, 0.946 mmol) were dissolved in water (2 mL). The resulting solution was added to a solution of N-(2,4-dichlorobenzyl)-5-formyl-N,3-dimethyl-4-(phenylsulfonyl)-1H-pyrrole-2-carboxamide, t-butanol (10 mL), 2-methyl-2 butene (2 mL), and tetrahydrofuran (2 mL). The reaction mixture was stirred 1 hour at room temperature. Water (20 mL) was added, and the reaction was extracted with ethyl acetate. The ethyl acetate layer was discarded. The aqueous phase was acidified with 1M HCl to give pH less than 2, and then extracted with ethyl acetate. The ethyl acetate phase was washed with saturated brine, dried over sodium sulfate and concentrated to give the title compound.

### Step 6: 1H-Indazole-3-carboxamide

Ethyl 1H-indazole-3-carboxylate (0.500 g, 2.62 mmol) was dissolved in methanolic ammonia (15 mL, 4N ammonia in methanol) and stirred 4 days at 110° C. in a sealed tube. The solvent was removed in vacuo to give the title compound.

### Step 7: 1H-Indazole-3-carbonitrile

1H-Indazole-3-carboxamide (0.400 g, 2.48 mmol) was dissolved in pyridine (4 mL) and dry dichloromethane (4 mL). Trifluoroacetic acid anhydride (0.863 mL, 6.20 mmol) was added and the reaction stirred at room temperature 10 minutes. The reaction was concentrated in vacuo and the residue taken up in ethyl acetate, then washed with water, saturated sodium bicarbonate and saturated brine. The organic phase was dried over sodium sulfate, filtered and concentrated to give the title compound.

### Step 8: 1-(1H-Indazol-3-yl)methanamine

1H-Indazole-3-carbonitrile (0.360 g, 2.51 mmol) was dissolved in methanol, and the solution purged with nitrogen. Raney nickel was added and the reaction stirred under 1 atm hydrogen at room temperature overnight. The catalyst was filtered and the filtrate concentrated to give the title compound.

### Step 9: N-(2,4-dichlorobenzyl)-N′-(1H-indazol-3-ylmethyl)-N,3-dimethyl-4-(phenylsulfonyl)-1H-pyrrole-2,5-dicarboxamide

The following were combined in dimethylformamide (1 mL) and stirred at room temperature overnight under inert atmosphere: 5-{[(2,4-dichlorobenzyl)(methyl)amino]carbonyl}-4-methyl-3-(phenylsulfonyl)-1H-pyrrole-2-carboxylic acid (0.010 g, 0.021 mmol), 1-(1H-indazol-3-yl)methanamine (0.0006 g, 0.042 mmol), N-hydroxybenzotriazole (0.006 g, 0.042 mmol), N-(3-dimethylaminopropyl) N′-ethylcarbodiimide hydrochloride (0.008 g, 0.042 mmol). The product was isolated by reverse phase HPLC using gradient elution with 0.1% trifluoroacetic acid in acetonitrile and 0.1% trifluoroacetic acid in water. The pure fractions were collected and concentrated to give the title compound. MS (M+1) 610.1069

### Examples 72-77

The compounds in Table E were prepared using a procedure similar to that employed in Example 71. The table provides the structure and name of each compound and the mass of its molecular ion plus 1 (M+1) as determined via MS. When the compound was prepared as a salt, the identity of the salt is included in parentheses following the compound name for the free base.

TABLE E

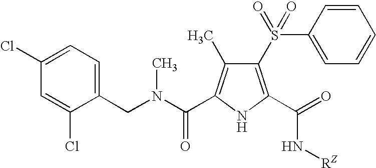

| Ex. | Compound | R^{Z} | M + 1 |
|---|---|---|---|
| 72 | N-(2,4-dichlorobenzyl)-N,N′,3- trimethyl-4-(phenylsulfonyl)-1H- pyrrole-2,5-dicarboxamide | CH₃ | 493.0629 |
| 73 | N-(2,4-dichlorobenzyl)-N,3- dimethyl-N′-[(3-methyl-4- pyridinyl)methyl]-4- (phenylsulfonyl)-1H-pyrrole-2,5- dicarboxamide (trifluoroacetic acid salt) | 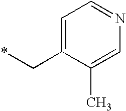 | 585.3 |
| 74 | N-(2,4-dichlorobenzyl)-N,3- dimethyl-4-(phenylsulfonyl)-N′-(2- pyridinylmethyl)-1H-pyrrole-2,5- dicarboxamide (trifluoroacetic acid salt) | 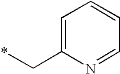 | 571.3 |
| 75 | N-(2,4-dichlorobenzyl)-N,3- dimethyl-4-(phenylsulfonyl)-N′-(3- pyridinylmethyl)-1H-pyrrole-2,5- dicarboxamide (trifluoroacetic acid salt) | 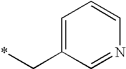 | 571.3 |
| 76 | N-(2,4-dichlorobenzyl)-N,3- dimethyl-4-(phenylsulfonyl)-N′- (1,3-thiazol-2-ylmethyl)-1H- pyrrole-2,5-dicarboxamide | 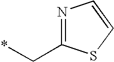 | 577.2 |
| 77 | N′-(2-chloro-6-fluorobenzyl)-N- (2,4-dichlorobenzyl)-N,3- dimethyl-4-(phenylsulfonyl)-1H- pyrrole-2,5-dicarboxamide | 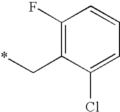 | 622.9 |

### Example 78

### 4-Methyl-3-(phenylsulfonyl)-5-(1,3,4,5-tetrahydro-2H-2-benzazepine-2-ylcarbonyl)-1H-pyrrole-2-carboxamide

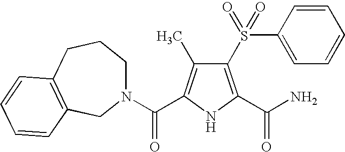

### Step 1: Ethyl 3,5-dimethyl-4-phenylsulfonyl-1H-pyrrole-2-carboxylate

Ethyl 4-(chlorosulfonyl)-3,5-dimethyl-1H-pyrrole-2-carboxylate (1.74 g, 6.55 mmol), benzene (0.875 mL, 9.82 mmol), and indium (III) chloride (101 mg, 0.485 mmol) were stirred in trifluoroacetic acid (9 mL). Trifluoromethysulfonic acid (0.698 mL, 7.86 mmol) was added to the solution and the resulting reaction was heated to 60° C. for 3 hours. The reaction was cooled to room temperature and quenched via dropwise addition into ice water. This was extracted with chloroform (2×). The combined organic layers were washed with saturated sodium bicarbonate, water, brine, dried over sodium sulfate, filtered and concentrated in vacuo. The residue was purified via flash chromatography on a 254 mm×40 mm silica gel column with 30% ethyl acetate/hexanes as eluant to afford the title compound.

### Step 2: 4-Methyl-3-(phenylsulfonyl)-5-(1,3,4,5-tetrahydro-2H-2-benzazepine-2-ylcarbonyl)-1H-pyrrole-2-carboxamide

Ethyl 3,5-dimethyl-4-phenylsulfonyl-1H-pyrrole-2-carboxylate from Step 1 above was converted to 5-ethoxycarbonyl-4-methyl-3-phenylsulfonyl-1H-pyrrole-2-carboxylic acid according to the procedures described in Example 2. The title compound was prepared from 5-ethoxycarbonyl-4-methyl-3-phenylsulfonyl-1H-pyrrole-2-carboxylic acid according to the method described in Example 40, except 2,3,4,5-tetrahydro-1H-2-benzazepine (prepared according to the procedure described by Meyers, A. I., Hutchings, R. H, Tetrahedron, 1993 (49) 9, 1807-1820) was employed as the secondary amine component. The title compound was isolated after purification by silica gel chromatography. MS (M+1) 438.1487.

### Example 79

### 4-Methyl-3-(3,5-dimethylphenylsulfonyl)-5-(1,3,4,5-tetrahydro-2H-2-benzazepine-2-ylcarbonyl)-1H-pyrrole-2-carboxamide

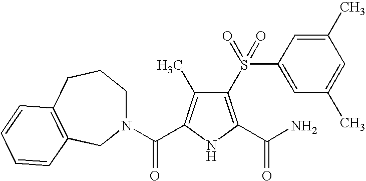

The title compound was prepared from 5-aminocarbonyl-4-[(3,5-dimethylphenyl)sulfonyl]-3-methyl-1H-pyrrole-2-carboxylic acid and 2,3,4,5-tetrahydro-1H-2-benzazepine according to the method described in Step 2, Example 78. The title compound was isolated after purification by chromatography on silica gel. MS (M+1) 466.1792

### Example 80

### 3-[(3,5-Dimethylphenyl)sulfonyl]-4-methyl-5-(4,6,7,8-tetrahydro-5H-thieno[3,2-c]azepin-5-ylcarbonyl)-1H-pyrrole-2-carboxamide

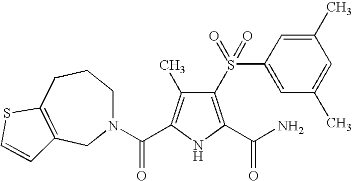

### Step 1: 6,7-Dihydro-1-benzothiophen-4(5H)-one oxime

To a solution of 6,7-dihydro-1-benzothiophen-4(5H)-one (2.00 g, 13.13 mmol) in ethanol (200 mL) was added a solution of hydroxylamine hydrochloride (4.56 g, 65.69 mmol) in 5 N sodium acetate (120 mL). The reaction mixture was stirred 2 hours at 100° C. The solvent was removed in vacuo and the crude product was dissolved in water and extracted with ethyl acetate. The organic phase was washed with saturated brine, dried over sodium sulfate and concentrated. The crude product was purified by silica gel chromatography using gradient elution (0 to 35% ethyl acetate in hexane) to give the title compound. MS (M+1) 472.1384

### Example 81

### 3-[(3,5-dichlorophenyl)sulfonyl]-4-methyl-5-(8-methoxy-1,3,4,5-tetrahydro-2H-2-benzazepin-2-ylcarbonyl)-1H-pyrrole-2-carboxamide

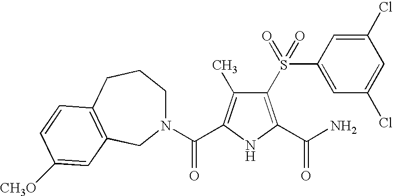

### Step 1: 8-methoxy-2,3,4,5-tetrahydro-benzo[C]-azepin-1-one

To an emulsion of 7-methoxy-tetralone (2.0 g, 11.35 mmol) in concentrated HCl (37%, 28.4 mL) at room temperature was added sodium azide (0.959 g, 14.75 mmol) in portions. The resulting mixture was stirred at room temperature overnight. The reaction was diluted with deionized water and brought to a pH of 10 with solid sodium carbonate. The basic aqueous solution was extracted with ethyl acetate (2×). The combined organic layers were washed with water, brine, dried over sodium sulfate, filtered and concentrated in vacuo. The residue was purified via flash chromatography on a 254 mm×40 mm silica gel column with 75% ethyl acetate/hexanes to 100% ethyl acetate as eluent to afford the title compound as a white solid.

### Step 2: 8-methoxy-2,3,4,5-tetrahydro-benzo[C]-azepine

8-methoxy-2,3,4,5-tetrahydro-benzo[C]-azepin-1-one (0.512 g, 2.68 mmol) was stirred in anhydrous tetrahydrofuran (13.4 mL) at room temperature under nitrogen. A solution of lithium aluminum hydride in tetrahydrofuran (1M, 4.02 mL) was added dropwise via syringe to the clear solution. The resulting reaction mixture was refluxed at 80° C. for 1.5 hours. The reaction was cooled to room temperature and then brought to 0° C. with an ice bath. This was quenched by slow addition of saturated potassium sodium tartrate. The biphase was stirred at room temperature for 30 minutes and extracted with ethyl acetate (2×). The combined organic layers were washed with water, brine, dried over sodium sulfate, filtered and concentrated in vacuo to yield the title compound as a clear oil.

### Step 3: 3-[(3,5-dichlorophenyl)sulfonyl]-4-methyl-5-(8-methoxy-1,3,4,5-tetrahydro-2H-2-benzazepin-2-ylcarbonyl)-1H-pyrrole-2-carboxamide

5-Aminocarbonyl-4-methyl-3-[(3,5-dichloro)phenylsulfonyl]-1H-pyrrole-2-carboxylic acid was prepared according to the procedures described in Example 2. The title compound was prepared from 5-Aminocarbonyl-4-methyl-3-[(3,5-dichloro)phenylsulfonyl]-1H-pyrrole-2-carboxylic acid according to the method described in Example 40, except 8-methoxy-2,3,4,5-tetrahydro-benzo[C]-azepine was employed as the secondary amine component. The title compound was isolated after purification by silica gel chromatography. MS (M+1) 536.0798.

### Example 82

### 3-[(3,5-dichlorophenyl)sulfonyl]-4-methyl-5-(8-hydroxy-1,3,4,5-tetrahydro-2H-2-benzazepin-2-ylcarbonyl)-1H-pyrrole-2-carboxamide

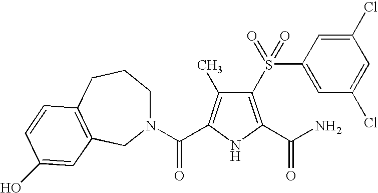

### Step 1: 3-[(3,5-dichlorophenyl)sulfonyl]-4-methyl-5-(8-hydroxy-1,3,4,5-tetrahydro-2H-2-benzazepin-2-ylcarbonyl)-1H-pyrrole-2-carboxamide

3-[(3,5-dichlorophenyl)sulfonyl]-4-methyl-5-(8-methoxy-1,3,4,5-tetrahydro-2H-2-benzazepin-2-ylcarbonyl)-1H-pyrrole-2-carboxamide (75 mg, 0.14 mmol) was stirred in anhydrous dichlormethane under nitrogen and chilled to 0° C. with an ice bath. Boron tribromide (280 μl, 1M/dichloromethane) was added and the resulting solution was stirred at room temperature for 18 h. The reaction was quenched with methanol and stirred for 30 minutes. This was concentrated in vacuo. The residue was purified via flash chromatography on a 254 mm×20 mm silica gel column with 2% to 3% methanol/dichloromethane as eluent to afford the title compound as a white solid. MS (M+1) 522.0642.

### Examples 83-95

Examples 83-95 in Table F were prepared using a procedure similar to that employed in Step 2, Example 78 and Examples 81-82. 5-Aminocarbonyl-4-[(3,5-dichlorophenyl)sulfonyl]-3-methyl-1H-pyrrole-2-carboxylic acid was prepared according to the methods described in Example 2. The table provides the structure and name of each compound and the mass of its molecular ion plus 1 (M+1) as determined via MS.

TABLE F

| Ex. | Compound | Structure | M + 1 |
|---|---|---|---|
| 83 | 3-[(3,5- dichlorophenyl)sulfonyl]-4- methyl-5-(1,3,4,5- tetrahydro-2H-2- benzazepin-2-ylcarbonyl)- 1H-pyrrole-2-carboxamide | 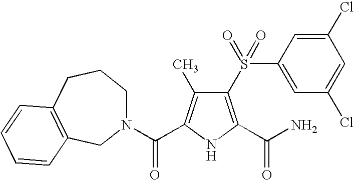 | 444.1067 |
| 84 | 3-[(3,5- dichlorophenyl)sulfonyl]-4- methyl-5-(4,6,7,8- tetrahydro-5H-thieno[3,2- c]azepin-5-ylcarbonyl)-1H- pyrrole-2-carboxamide | 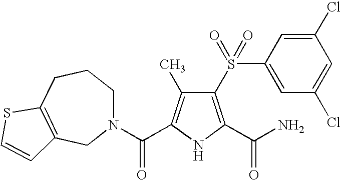 | 512.0255 |
| 85 | 3-[(3,5- dichlorophenyl)sulfonyl]-4- methyl-5-(7-methoxy- 1,3,4,5-tetrahydro-2H-2- benzazepin-2-ylcarbonyl)- 1H-pyrrole-2-carboxamide | 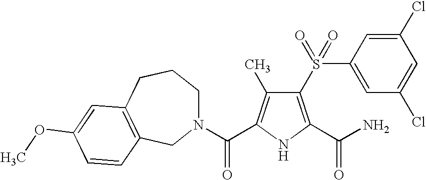 | 536.0831 |
| 86 | 3-[(3,5- dichlorophenyl)sulfonyl]-4- methyl-5-(7-hydroxy- 1,3,4,5-tetrahydro-2H-2- benzazepin-2-ylcarbonyl)- 1H-pyrrole-2-carboxamide | 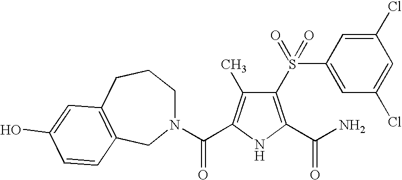 | 522.0578 |
| 87 | 3-[(3,5- dimethylphenyl)sulfonyl]- 4-methyl-5-(7-methoxy- 1,3,4,5-tetrahydro-2H-2- benzazepin-2-ylcarbonyl)- 1H-pyrrole-2-carboxamide | 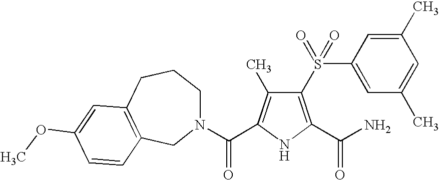 | 496.1902 |
| 88 | 3-[(3,5- dimethylphenyl)sulfonyl]- 4-methyl-5-(7-hydroxy- 1,3,4,5-tetrahydro-2H-2- benzazepin-2-ylcarbonyl)- 1H-pyrrole-2-carboxamide | 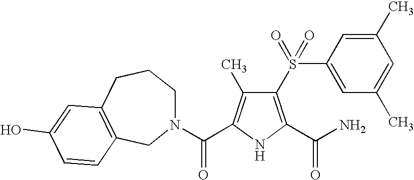 | 482.1753 |
| 89 | 3-[(3,5- dimethylphenyl)sulfonyl]- 4-methyl-5-(8-methoxy- 1,3,4,5-tetrahydro-2H-2- benzazepin-2-ylcarbonyl)- 1H-pyrrole-2-carboxamide | 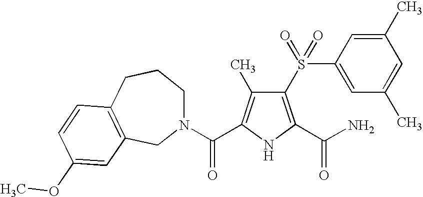 | 496.1902 |
| 90 | 3-[(3,5- dimethylphenyl)sulfonyl]- 4-methyl-5-(8-hydroxy- 1,3,4,5-tetrahydro-2H-2- benzazepin-2-ylcarbonyl)- 1H-pyrrole-2-carboxamide | 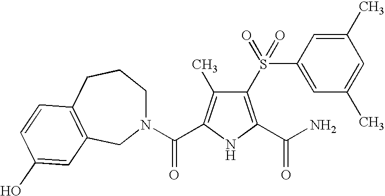 | 482.1732 |
| 91 | 3-(phenylsulfonyl)-4- methyl-5-(8-methoxy- 1,3,4,5-tetrahydro-2H-2- benzazepin-2-ylcarbonyl)- 1H-pyrrole-2-carboxamide | 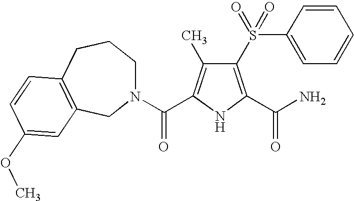 | 468.1593 |
| 92 | 3-(phenylsulfonyl)-4- methyl-5-(8-hydroxy- 1,3,4,5-tetrahydro-2H-2- benzazepin-2-ylcarbonyl)- 1H-pyrrole-2-carboxamide | 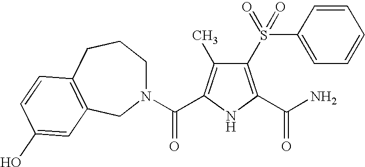 | 454.1443 |
| 93 | 3-[(3- fluorophenyl)sulfonyl]-4- methyl-5-(8-methoxy- 1,3,4,5-tetrahydro-2H-2- benzazepin-2-ylcarbonyl)- 1H-pyrrole-2-carboxamide | 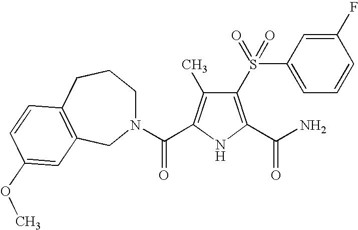 | 486.1493 |
| 94 | 3-[(3- fluorophenyl)sulfonyl]-4- methyl-5-(8-hydroxy- 1,3,4,5-tetrahydro-2H-2- benzazepin-2-ylcarbonyl)- 1H-pyrrole-2-carboxamide | 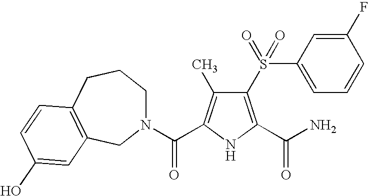 | 472.1352 |
| 95 | 3-[(3-(1,1,1- trifluoromethyl) phenyl)sulfonyl]-4-methyl- 5-(8-methoxy-1,3,4,5- tetrahydro-2H-2- benzazepin-2-ylcarbonyl)- 1H-pyrrole-2-carboxamide | 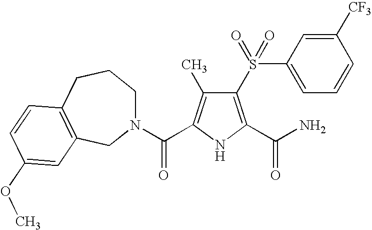 | 536.1464 |

### Example 96

### 3-[(3,5-Dichlorophenyl)sulfonyl]-4-methyl-5-[(2-chloro-4,6,7,8-tetrahydro-5H-thieno[3,2-c]azepin-5-yl)carbonyl]-1H-pyrrole-2-carboxamide

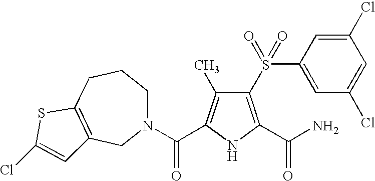

### Step 1: (4E)-6,7-dihydro-1-benzothiophen-4(5H)-one oxime

To a solution of 6,7-dihydro-1-benzothiophen-4(5H)-one (2.00 g, 13.13 mmol) in ethanol (200 mL) was added a solution of hydroxylamine hydrochloride (4.56 g, 65.69 mmol) in 5 N sodium acetate (120 mL). The reaction mixture was stirred 2 hours at 100° C. The solvent was removed in vacuo and the crude product was dissolved in water and extracted with ethyl acetate. The organic phase was washed with saturated brine, dried over sodium sulfate and concentrated. The crude product was purified by silica gel chromatography using gradient elution (0 to 35% ethyl acetate in hexane) to give the title compound.

### Step 2: 4,6,7,8-tetrahydro-5H-thieno[3,2-b]azepin-5-one

Phosphorus pentoxide (11.30 g, 79.53 mmol) was added to methanesulfonic acid (10.92 g, 113.61 mmol) while stirring and the stirring was continued for 2 hour. The (4E)-6,7-dihydro-1-benzothiophen-4(5H)-one oxime (1.90 g, 11.36 mmol) was then added to the above stirred solution at 100° C. After stirring for 4 hours at 110° C. oil bath, the reaction mixture was cooled and quenched carefully with adding 10 ml saturated sodium bicarbonate. The mixture was extracted with chloroform (50 ml×2). This combined chloroform solution was washed with saturated sodium bicarbonate, water, saturated brine, dried with sodium sulfate, filtered and concentrated in vacuo. The residue was purified via flash chromatography on silica gel column (80 g) with 25% to 65% ethyl acetate/hexane gradient elution to separate the title compound.

### Step 3: 5,6,7,8-tetrahydro-4H-thieno[3,2-c]azepine

The 4,6,7,8-tetrahydro-5H-thieno[3,2-b]azepin-5-one (0.75 g, 4.48 mmol was dissolved in 20 ml dry THF, to it was added the lithium aluminum hydride (1N in THF) (6.73 ml, 6.73 mmol) The resulting mixture was stirred at 60° C. for 3 hours. The reaction mixture was cooled to room temperature, then placed in an ice bath. The reaction was quenched by adding water drop wise until no more bubble formed. It was stirred for another 15 minutes then filtered by syringe filter. The filter liquid was concentrated to give the title compound.

### Step 4: 2-chloro-5,6,7,8-tetrahydro-4H-thieno[3,2-c]azepine

The 5,6,7,8-tetrahydro-4H-thieno[3,2-c]azepine (30 mg, 0.20 mmol) was dissolved in acetic acid (2 ml), to it was added the perchloric acid (2.81 mg, 0.02 mmol) and N-chlorosuccinimide (31 mg, 0.24 mmol). The resulting mixture was stirred over night at 60° C. The acetic acid was evaporated in vacuo and the residue was dissolved in 1 ml methanol and purified by Gilson reverse phase HPLC to give the title compound.

### Step 5: 3-[(3,5-Dichlorophenypsulfonyl]-4-methyl-5-[(2-chloro-4,6,7,8-tetrahydro-5H-thieno[3,2-c]azepin-5-yl)carbonyl]-1H-pyrrole-2-carboxamide

The title compound was obtained from 2-chloro-5,6,7,8-tetrahydro-4H-thieno[3,2-c]azepine according to the procedure described in Example 40. MS (M+1) 545.9873

### Example 97

### 3-[(3,5-Dichlorophenypsulfonyl]-4-methyl-5-[(2-(methylsulfonyl)-4,6,7,8-tetrahydro-5H-thieno[3,2-c]azepin-5-yl)carbonyl]-1H-pyrrole-2-carboxamide

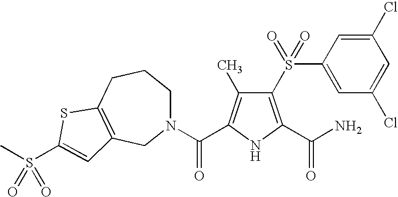

### Step 1: 2-(methylthio)-5,6,7,8-tetrahydro-4H-thieno[3,2-c]azepine

The 5,6,7,8-tetrahydro-4H-thieno[3,2-c]azepine (prepared in example 96, step 1-3) (0.25 g, 1.57 mmol) was dissolved in 10 ml dry THF. The resulting mixture was cooled to −80° C. with dry ice/acetone bath, and to it was added n-butyl lithium (0.22 g, 3.45 mmol) in cyclohexane N). The resulting mixture was stirred at −80° C. for 1 hour, methyl disulfide (0.74 g, 7.83 mmol) was added in, and then allowed to worm to room temperature and stirred over night. The solvent with extra reagent was taken off by vacuum and the crude product was suspended in ethyl acetate (30 mL) and washed with brine, dried over sodium sulfate and concentrated. The crude product was purified with reverse phase HPLC to give the title compound.

### Step 2: 3-[(3,5-Dichlorophenypsulfonyl]-4-methyl-5-[(2-(methylsulfonyl)-4,6,7,8-tetrahydro-5H-thieno[3,2-c]azepin-5-yl)carbonyl]-1H-pyrrole-2-carboxamide

3-[(3,5-Dichlorophenyl)sulfonyl]-4-methyl-5-[(2-(methylthio)4,6,7,8-tetrahydro-5H-thieno[3,2-c]azepin-5-yl)carbonyl]-1H-pyrrole-2-carboxamide was prepared according to the procedure described in Example 40. It was oxidized to the title compound following the reaction in Example 1, Step 2. The title compound was purified by reverse phase HPLC (gradient 0.1% TFA/acetonitrile and 0.1% aq. TFA). MS (m+1) 590.0023.

### Example 98

### 3-[(3,5-Dimethylphenyl)sulfonyl]-4-methyl-5-[(2-(methylsulfonyl)-4,6,7,8-tetrahydro-5H-thieno[3,2-c]azepin-5-yl)carbonyl]-1H-pyrrole-2-carboxamide

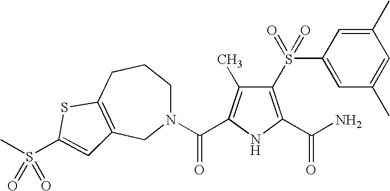

The title compound was prepared according to the procedure described in Example 97, except 5-aminocarbonyl-3-methyl-4-[(3,5-dimethylphenyl)sulfonyl-1H-pyrrole-2-carboxylic acid was employed in place of 5-aminocarbonyl-3-methyl-4-[(3,5-dichlorophenyl)sulfonyl-1H-pyrrole-2-carboxylic acid. After purification, the title compound was obtained. MS (M+1) 550.1121.

### Example 99-114

Examples 99-104 in Table G below were prepared using a procedure similar to that employed in Example 41, steps 1-2, and Example 40 to prepare the protected compounds. The pure protected compounds were dissolved in a mixture of dichloromethane/trifluoroacetic acid (1/1) and stirred for 30 minutes. Evaporation of solvent and TFA provided the compounds in Examples 99-104.

Examples 105-114 in Table G below were prepared using a procedure similar to that employed in Example 4, Step 1 and Example 40.

The table provides the structure and name (free base) of each compound (TFA salt) and the mass of its molecular ion plus 1 (M+1) as determined via MS.

TABLE G

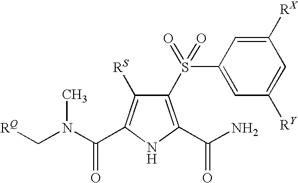

| Ex. | Compound | R^{Q} | R^{S} | R^{X} | R^{Y} | M + 1 |
|---|---|---|---|---|---|---|
| 99 | N5-[(2-aminopyridin-4- yl)methyl]-3-[(3- fluorophenyl)sulfonyl]-4- isopropyl-N5-methyl-1H- pyrrole-2,5- dicarboxamide | 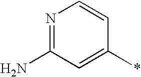 | i-Pr | F | H | 474.1601 |
| 100 | N5-[(2-aminopyridin-4- yl)methyl]-3-[(3,5- difluorophenyl)sulfonyl]- 4-isopropyl-N5-methyl- 1H-pyrrole-2,5- dicarboxamide | 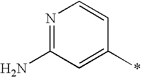 | i-Pr | F | F | 492.1502 |
| 101 | N5-[(2-aminopyridin-4- yl)methyl]-3- phenylsulfonyl]-4- isopropyl-N5-methyl-1H- pyrrole-2,5- dicarboxamide | 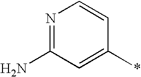 | i-Pr | H | H | 456.1681 |
| 102 | N5-[(2-aminopyridin-4- yl)methyl]-3-[(3,5- dimethylphenyl)sulfonyl]- 4-isopropyl-N5-methyl- 1H-pyrrole-2,5- dicarboxamide | 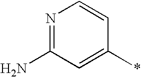 | i-Pr | CH₃ | CH₃ | 484.2034 |
| 103 | N5-[(2-amino-5- fluoropyridin-4- yl)methyl]-3-[(3,5- dimethylphenyl)sulfonyl]- N5,4-dimethyl-1H- pyrrole-2,5- dicarboxamide | 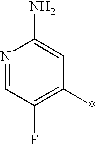 | CH₃ | CH₃ | CH₃ | 474.1621 |
| 104 | N2-[(3-N5-[(2-amino-5- fluoropyridin-4- yl)methyl]-3-isopropyl- N2-methyl-4- (phenylsulfonyl)-1H- pyrrole-2,5- dicarboxamide | 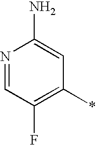 | i-Pr | H | H | 474.1533 |
| 105 | N5-[(3-chloropyridin-4- yl)methyl]-3-[(3,5- dimethylphenyl)sulfonyl]- N5,4-dimethyl-1H- pyrrole-2,5- dicarboxamide(free base) | 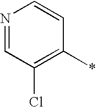 | CH₃ | CH₃ | CH₃ | 475.1180 |
| 106 | N2-[(3-chloropyridin-4- yl)methyl]-3-isopropyl- N2-methyl-4- (phenylsulfonyl)-1H- pyrrole-2,5- dicarboxamide | 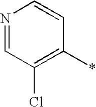 | i-Pr | H | H | 475.1180 |
| 107 | N5-[(2-chloro-3- fluoropyridin-4- yl)methyl]-3-[(3,5- dimethylphenyl)sulfonyl]- N5,4-dimethyl-1H- pyrrole-2,5- dicarboxamide | 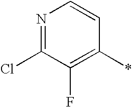 | CH₃ | CH₃ | CH₃ | 493.1107 |
| 108 | N2-[(2-chloro-3- fluoropyridin-4- yl)methyl]-3-isopropyl- N2-methyl-4- (phenylsulfonyl)-1H- pyrrole-2,5- dicarboxamide | 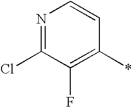 | i-Pr | H | H | 493.1124 |
| 109 | 3-[(3,5- dimethylphenyl)sulfonyl]- N5-[(3-fluoropyridin-4- yl)methyl]-N5,4- dimethyl-1H-pyrrole-2,5- dicarboxamide | 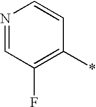 | CH₃ | CH₃ | CH₃ | 459.1459 |
| 110 | N2-[(3-fluoropyridin-4- yl)methyl]-3-isopropyl- N2-methyl-4- (phenylsulfonyl)-1H- pyrrole-2,5- dicarboxamide | 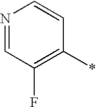 | i-Pr | H | H | 459.1495 |
| 111 | N5-[(3,5-difluoropyridin- 4-yl)methyl]-3-[(3,5- dimethylphenyl)sulfonyl]- N5,4-dimethyl-1H- pyrrole-2,5- dicarboxamide | 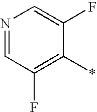 | CH₃ | CH₃ | CH₃ | 477.1395 |
| 112 | N2-[(3,5-difluoropyridin- 4-yl)methyl]-3-isopropyl- N2-methyl-4- (phenylsulfonyl)-1H- pyrrole-2,5- dicarboxamide | 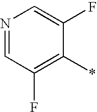 | i-Pr | H | H | 477.1388 |
| 113 | N5-[(2-chloro-5- fluoropyridin-4- yl)methyl]-3-[(3,5- dimethylphenyl)sulfonyl]- N5,4-dimethyl-1H- pyrrole-2,5- dicarboxamide | 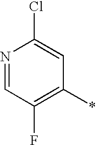 | CH₃ | CH₃ | CH₃ | 493.1109 |
| 114 | N2-[(2-chloro-5- fluoropyridin-4- yl)methyl]-3-isopropyl- N2-methyl-4- (phenylsulfonyl)-1H- pyrrole-2,5- dicarboxamide | 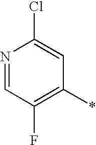 | i-Pr | H | H | 493.1107 |

### Example 115

N2-[(2-amino-3-fluoropyridin-4-yl)methyl]-3-isopropyl-N2-methyl-4-(phenylsulfonyl)-1H-pyrrole-2,5-dicarboxamide

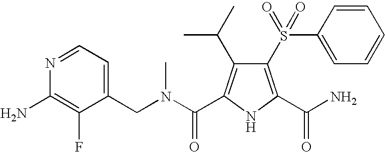

### Step 1: 2,3-difluoro-N-methylisonicotinamide

A solution of 2,3-difluoroisonicotinic acid (0.994 g, 6.25 mmol), N-hydroxybenzotriazole (1.244 g, 8.12 mmol), N-(3-dimethylaminopropyl) N′-ethylcarbodiimide hydrochloride (1.198 g, 6.25 mmol) and 6 ml methanamine (2N in MeOH) in dimethylformamide (5 mL) was stirred over night. The reaction mixture was poured to 30 mL cold water and extracted with EtOAc (50 mL×2). The combined EtOAc solution was washed with brine, dried over sodium sulfate and concentrated to give the title compound.

### Step 2: 3-fluoro-2-[(4-methoxybenzyl)amino]-N-methylisonicotinamide

A solution of 2,3-difluoro-N-methylisonicotinamide (0.822 g, 4.78 mmol), 4-methoxybenzylamine (0.983 g, 7.16 mmol), and potassium carbonate (0.990 g, 7.16 mmol) in 15 mL dimethyl sulfoxide was sealed and stirred at 120° C. for 1 hour. The reaction mixture was cooled to room temperature, and then poured to 50 mL cold water and extracted with EtOAc (50 mL×2). The combined ethyl acetate solution was washed with brine, dried over sodium sulfate and concentrated. The title compound was purified via flash chromatography on silica gel column (40 g) with 25% to 65% ethyl acetate/hexane gradient elution to separate the title compound.

### Step 3: 3-fluoro-N-(4-methoxybenzyl)-4-[(methylamino)methyl]pyridin-2-amine

The 3-fluoro-2-[(4-methoxybenzyl)amino]-N-methylisonicotinamide (0.100 g, 0.346 mmol) was dissolved in 0.5 mL dry THF, to it was added the borane-methyl sulfide (0.691 ml, 1.383 mmol) in THF (2M) and the resulting solution was stirred at 60° C. for 2 hours. The reaction was quenched by adding few drops of water, then 1 mL 1N HCl and stirred over night. It was extracted with ethyl acetate (5 mL×2). The solvent was taken off and the residue was dissolved in 1 mL DMF and purified with reverse phase HPLC to give the title compound.

### Step 4: N2-[(2-amino-3-fluoropyridin-4-yl)methyl]-3-isopropyl-N2-methyl-4-(phenylsulfonyl)-1H-pyrrole-2,5-dicarboxamide

The protected compound was prepared using a procedure similar to that employed in Example 40. The pure protected compound was dissolved in the mixture of dichloromethane/trifluoroacetic acid (1/1) and stirred at 60° C. for 2 hours. The title compound was further purified with reverse phase HPLC. MS (M+1) 474.1533.

### Example 116

### N-(2,4-dichlorobenzyl)-N,3-dimethyl-4-(1-pyrrolidinylsulfonyl)-1H-pyrrole-2,5-dicarboxamide

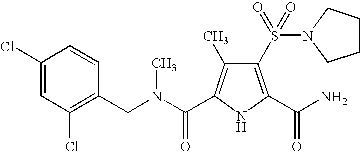

### Step 1: Ethyl 3,5-dimethyl-4-(pyrrolidin-1-ylsulfonyl)-1H-pyrrole-2-carboxylate

To a solution of ethyl 4-(chlorosulfonyl)-3,5-dimethyl-1H-pyrrole-2-carboxylate (2.06 g, 7.75 mmol) in anhydrous dichloromethane (60 mL) was added pyrrolidine (1.27 mL, 15.5 mmol) via syringe. This was stirred at room temperature 1 hour. The reaction was diluted with dichloromethane and washed with 1N HCl, water, brine, dried with sodium sulfate, filtered and concentrated in vacuo. The residue was triturated with ether to afford the title compound as an off-white solid.

### Step 2: Ethyl 5-formyl-3-methyl-4-(pyrrolidin-1-ylsulfonyl)-1H-pyrrole-2-carboxylate

To a suspension of ethyl 3,5-dimethyl-4-(pyrrolidin-1-ylsulfonyl)-1H-pyrrole-2-carboxylate (2.00 g, 6.64 mmol) in anhydrous dichloromethane (60 mL) was added dropwise sulfuryl chloride (1.62 mL, 19.97 mmol). The resulting solution was stirred at room temperature 1 hour. The reaction was concentrated in vacuo and diluted with 1:1 acetone:water (60 mL). The solution was heated to reflux for 15 minutes. The acetone was removed in vacuo and the aqueous layer was extracted with dichloromethane. The layers were separated and the organic layer was washed with brine, dried over sodium sulfate, filtered and concentrated in vacuo to give the title compound.

### Step 3: 5-(Ethoxycarbonyl)-4-methyl-3-(pyrrolidin-1-ylsulfonyl)-1H-pyrrole-2-carboxylic acid

Ethyl 5-formyl-3-methyl-4-(pyrrolidin-1-ylsulfonyl)-1H-pyrrole-2-carboxylate (2.00 g, 6.36 mmol) was dissolved in minimal tetrahydrofuran (10 mL). To this stirred solution was added t-butanol (20 mL) and 2-methyl-2-butene (5 mL). A solution of sodium chlorite (0.690 g, 7.63 mmol) and sodium dihydrogen phosphate (0.840 g, 7.00 mmol) in deionized water (30 mL) was added to the reaction. The resulting mixture was stirred at room temperature 2 hours. The organic volatiles were removed in vacuo and the remaining aqueous phase was extracted with dichloromethane. The layers were separated and the organic layer was washed with brine, dried over sodium sulfate, filtered and concentrated in vacuo to give the title compound.

### Step 4: Ethyl 5-(aminocarbonyl)-3-methyl-4-(pyrrolidin-1-ylsulfonyl)-1H-pyrrole-2-carboxylate

5-(Ethoxycarbonyl)-4-methyl-3-(pyrrolidin-1-ylsulfonyl)-1H-pyrrole-2-carboxylic acid (0.500 g, 1.51 mmol), N-(3-dimethylaminopropyl) N′-ethylcarbodiimide hydrochloride (0.363 g, 1.89 mmol), and N-hydroxybenzotriazole (0.278 g, 1.81 mmol) were stirred in anhydrous acetonitrile (10 mL) under nitrogen. To this resulting solution was added ammonium hydroxide (29%, 0.365 mL, 3.03 mmol) and a white precipitate formed. This was stirred 15 minutes at room temperature. The reaction was concentrated in vacuo and the residue was partitioned between water and dichloromethane. The layers were separated and the organic layer was washed with brine, dried over sodium sulfate, filtered and concentrated in vacuo to give the title compound.

### Step 5: 5-(Aminocarbonyl)-3-methyl-4-(pyrrolidin-1-ylsulfonyl)-1H-pyrrole-2-carboxylic acid

Ethyl 5-(aminocarbonyl)-3-methyl-4-(pyrrolidin-1-ylsulfonyl)-1H-pyrrole-2-carboxylate (0.400 g, 1.21 mmol) was stirred in 1,2-dimethoxyethane (12 mL). 1N lithium hydroxide (12 mL, 12 mmol) was added to this solution and the resulting mixture was heated to 80° C. for 5 hours. The organics were removed in vacuo and the remaining aqueous phase was brought to a pH of 3 with 1N HCl. This was extracted with ethyl acetate (2×). The combined organic layers were washed with water, brine, dried over sodium sulfate, filtered and concentrated in vacuo to give the title compound.

### Step 6: N-(2,4-Dichlorobenzyl)-N,3-dimethyl-4-(1-pyrrolidinylsulfonyl)-1H-pyrrole-2,5-dicarboxamide

5-(Aminocarbonyl)-3-methyl-4-(pyrrolidin-1-ylsulfonyl)-1′-1-pyrrole-2-carboxylic acid (41 mg, 0.14 mmol), N-(3-dimethylaminopropyl) N′-ethylcarbodiimide hydrochloride (52 mg, 0.27 mmol), and N-hydroxybenzotriazole (42 mg, 0.27 mmol) were stirred in anhydrous acetonitrile (1 mL) under nitrogen. Triethylamine (0.036 mL, 0.27 mmol) was added to the reaction followed by 2,4-dichlorobenzyl-N-methyl amine hydrochloride (46 mg, 0.20 mmol). The resulting mixture was stirred overnight at room temperature. The reaction was concentrated in vacuo and the residue was partitioned between water and ethyl acetate. The layers were separated and the organic layer was washed with brine, dried over sodium sulfate, filtered and concentrated in vacuo. The title compound was purified via flash chromatography on a silica gel column (50×20 mm) with 2% methanol:dichloromethane as eluant. MS (M+1)=473.0801.

### Example 117

### N-(2-chlorobenzyl)-N,3-dimethyl-N′-(2-pyridinylmethyl)-4-(1-pyrrolidinylsulfonyl)-1H-pyrrole-2,5-dicarboxamide

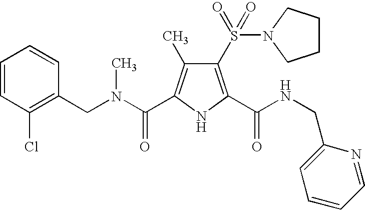

The title compound was prepared in the same way as described for N-(2,4-dichlorobenzyl)-N,3-dimethyl-4-(1-pyrrolidinylsulfonyl)-1H-pyrrole-2,5-dicarboxamide, except Step 4 utilized 5-(ethoxycarbonyl)-4-methyl-3-(pyrrolidin-1-ylsulfonyl)-1H-pyrrole-2-carboxylic acid (150 mg, 0.45 mmol), N-(3-dimethylaminopropyl) N′-ethylcarbodiimide hydrochloride (109 mg, 0.57 mmol), and N-hydroxybenzotriazole (87 mg, 0.57 mmol) with 2-pyridylmethylamine (98 mg, 0.91 mmol) instead of ammonium hydroxide, and Step 6 employed 2-chlorobenzyl-N-methylamine in place of 2,4-chlorobenzyl-N-methylamine hydrochloride. The title compound was purified via flash chromatography. MS (M+1)=530.1621.

### Examples 118-119

The compounds in Table H below were prepared using a procedure similar to that employed in Example 116. The table provides the structure and name of each compound and the mass of its molecular ion plus 1 (M+1) as determined via MS. When the compound was prepared as a salt, the identity of the salt is included in parentheses following the compound name for the free base.

TABLE H

| Ex. | Compound | Structure | M + 1 |
|---|---|---|---|
| 118 | N-(2,4-dichlorobenzyl)-N,3- dimethyl-4-(1- piperidinylsulfonyl)-1H-pyrrole- 2,5-dicarboxamide | 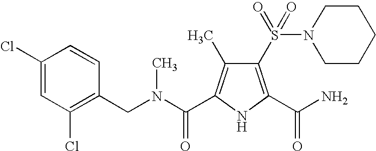 | 487.0969 |
| 119 | N-(2-chlorobenzyl)-N,3-dimethyl- 4-(1-piperidinylsulfonyl)-1H- pyrrole-2,5-dicarboxamide | 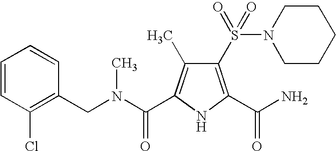 | 453.1343 |

### Example 120

### Encapsulated Oral Compositions

A capsule formulation suitable for use in the present invention can be prepared by filling standard two-piece gelatin capsules each with 100 mg of the title compound of Example 1, 150 mg of lactose, 50 mg of cellulose, and 3 mg of stearic acid. Encapsulated oral compositions containing any one of the title compounds of Examples 2 to 119 can be similarly prepared.

### Example 121

### SPA Assay for Inhibition of HIV Reverse Transcriptase

An assay to determine the in vitro inhibition of HIV reverse transcriptase by compounds of the present invention was conducted as follows: HIV-1 RT enzyme (1 nM) was combined with inhibitor or DMSO (10%) in assay buffer (50 mM Tris-HCl, pH 7.8, 1 mM dithiothreitol, 6 mM MgCl₂, 80 mM KCl, 0.025% CHAPS, 0.1 mM EGTA), and the mixture preincubated for 30 minutes at room temperature in microtiter Optiplates (Packard). 100 μL reaction mixtures were initiated with a combination of primer-template substrate (10 nM final concentration) and dNTPs (0.6 μM dNTPs, 0.75 μM [³H]-dGTP). The heterodimeric nucleic acid substrate was generated by annealing the DNA primer pD500 (described in Shaw-Reid et al., J. Biol. Chem., 278: 2777-2780; obtained from Integrated DNA Technologies) to t500, a 500 nucleotide RNA template created by in vitro transcription (see Shaw-Reid et al., J. Biol. Chem., 278: 2777-2780). After 1 hour incubation at 37° C., reactions were quenched by 10 μL streptavidin scintillation proximity assay beads (10 mg/mL, from Amersham Biosciences) in 0.5 M EDTA, pH 8. Microtiter plates were incubated an additional 10 minutes at 37° C. prior to quantification via Topcount (Packard). Representative compounds of the present invention exhibit inhibition of the reverse transcriptase enzyme in this assay. For example, the title compounds set forth above in Examples 1-11, 13-15, 20, 21, 25-27, 32, 34, 36-39, 43, 44, 54, 57, 71-77, 116 and 117 were tested in the assay and all were found to have IC₅₀ values of less than 5 micromolar.

Analogous assays were conducted substituting mutant HIV strains to determine the in vitro inhibition of compounds of the present invention against mutant HIV reverse transcriptase. In one strain the reverse transcriptase has the Y181C mutation and in the other strain the reverse transcriptase has the K103N mutation. The mutations were generated with the QUIKCHANGE site-directed mutagenesis kit (Stratagene). Representative compounds of the present invention exhibit inhibition of the reverse transcriptase enzyme in these assays. For example, the title compounds set forth above in Examples 1-11, 13-15, 20, 21, 25-27, 32, 34, 36-39, 43, 44, 54, 57, 71-77 and 116-119 were tested in the assays and were found to have IC₅₀ values of less than 8 micromolar in the Y181C assay and of less than 5 micromolar in the K103N assay.

### Example 122

### ECL Assay for Inhibition of HIV Reverse Transcriptase

Another assay to determine the in vitro inhibition of HIV reverse transcriptase by compounds of the present invention was conducted as follows: HIV-1 RT enzyme (0.1 nM) was combined with inhibitor or DMSO (10%) in assay buffer (50 mM Tris-HCl, pH 7.8, 1 mM dithiothreitol, 6 mM MgCl₂, 80 mM KCl, 0.025% CHAPS, 0.1 mM EGTA), and the mixture preincubated for 30 minutes at room temperature in microtiter plates (Costar #3359). 100 μL reaction mixtures were initiated with a combination of primer-template substrate (10 nM final concentration) and dNTPs (0.6 μM dNTPs, 1.25 μM BrdUTP). The heterodimeric nucleic acid substrate was generated by annealing the DNA primer pD500 (described in Shaw-Reid et al., J. Biol. Chem., 278: 2777-2780; obtained from Integrated DNA Technologies) to t500, a 500 nucleotide RNA template created by in vitro transcription (see Shaw-Reid et al., J. Biol. Chem., 278: 2777-2780). After 1 hour incubation at 37° C., reactions were quenched by 10 μL of 1 N NaOH. Microtiter plates were incubated for an additional 30 minutes at room temperature and then neutralized with 10 μL of 1 N HCl. A mixture of detection buffer containing ruthenylated anti-BrdU antibody and streptavidin coated magnetic beads were added to the plate and incubated at room temperature for 1.5 hours prior to quantification via electrochemiluminescence instrument. Representative compounds of the present invention exhibit inhibition of the reverse transcriptase enzyme in this assay. For example, the title compounds set forth above in Examples 12, 16-19, 22-24, 26, 28-31, 33, 35, 40-42, 45-53, 55, 56, 58-70, 70A, 70B, 78-84 and 86-115 were tested in the assay and all were found to have IC₅₀ values of less than 5 micromolar. (The compound of Example 85 was not tested in this assay.)

Analogous assays were conducted substituting mutant HIV strains to determine the in vitro inhibition of compounds of the present invention against mutant HIV reverse transcriptase. In one strain the reverse transcriptase has the Y181C mutation and in the other strain the reverse transcriptase has the K103N mutation. The mutations were generated with the QUIKCHANGE site-directed mutagenesis kit (Stratagene). Representative compounds of the present invention exhibit inhibition of the reverse transcriptase enzyme in these assays. For example, the title compounds set forth above in Examples 12, 16-19, 22-24, 26, 28-31, 33, 35, 40-42, 45-53, 55, 56, 58-70, 70A, 70B, 78-84 and 86-115 were tested in the assays and were found to have IC₅₀ values of less than 8 micromolar in the Y181C assay and of less than 5 micromolar in the K103N assay. (The compound of Example 85 was not tested in these assays.)

### Example 123

### Assay for Inhibition of HIV Replication

Assays for the inhibition of acute HIV-1 infection of T-lymphoid cells (alternatively referred to herein as the “spread assay”) were conducted in accordance with Vacca, J. P. et al., Proc. Natl. Acad. Sci. USA 1994, 91: 4096. The assays (using 10% FBS) tested for inhibition of wild type HIV-1 and of HIV strains containing the Y181C or K103N mutation. Representative compounds of the present invention exhibit inhibition of HIV replication in the assay employing wild-type HIV-1 and the mutant strains. For example, the compounds set forth in Examples 1 to 119 (including Examples 70A and 70B) were found to have CIC₉₅ values of less than 1000 nanomolar in the assay employing the wild type strain. The compounds of Examples 1-5, 7-70, 70A, 70B, 71-77 and 79-119 exhibited CIC₉₅ values of less than 8000 nanomolar in the assay employing the Y181C mutant strain. (Note that the compound of Example 6 was tested only up to a 833 nM concentration and the CIC₉₅ value was determined to be >833 nM. The compound of Example 78 was not tested in the Y1818C assay.) The compounds of Examples 1 to 119 (including Examples 70A and 70B) had CIC₉₅ values of less than 1000 nanomolar in the assay employing the K103N mutant strain. In an analogous assay employing a mutant strain containing both the K103N and Y181C mutations, the compounds of Examples 70A, 70B and 78-115 had CIC₉₅ values of less than 1000 nanomolar.

### Example 124

### Cytotoxicity

Cytotoxicity was determined by microscopic examination of the cells in each well in the spread assay, wherein a trained analyst observed each culture for any of the following morphological changes as compared to the control cultures: pH imbalance, cell abnormality, cytostatic, cytopathic, or crystallization (i.e., the compound is not soluble or forms crystals in the well). The toxicity value assigned to a given compound is the lowest concentration of the compound at which one of the above changes is observed. Representative compounds of the present invention exhibit no cytotoxicity at concentrations of up to 8 micromolar. In particular, the compounds set forth in Examples 1 to 119 exhibited no cytotoxicity at concentrations of up to 8 micromolar.

While the foregoing specification teaches the principles of the present invention, with examples provided for the purpose of illustration, the practice of the invention encompasses all of the usual variations, adaptations and/or modifications that come within the scope of the following claims.

1. A compound of Formula I, or a pharmaceutically acceptable salt thereof: 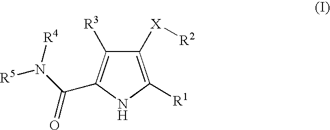
wherein:
X is S, S(O), S(O)₂, P(O)—OT, P(S)—OT, or P(N—U)—OT;
T is H or independently has the same definition as R²;
U independently has the same definition as R^{K};
R¹ is C(O)NR^{K}R^{L};
one of R^{K} and R^{L} is H, and the other of R^{K} and R^{L} is:
(1) H,
(2) C₁₋₆ alkyl,
(3) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(4) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(5) CycA,
(6) AryA,
(7) HetA,
(8) C₁₋₆ alkyl substituted with CycA, AryA, or HetA, or
(9) C₁₋₆ alkyl substituted with Y¹-CycA, Y¹-AryA, or Y¹-HetA;
R² is:
(1) C₁₋₆ alkyl,
(2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(4) CycB,
(5) AryB,
(6) HetB,
(7) C₁₋₆ alkyl substituted with CycB, AryB, or HetB,
(8) N(R^{A})R^{B},
(9) N(R^{A})—C₁₋₆ alkyl, wherein the alkyl is substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B}, with the proviso that OH, O—C₁₋₆ alkyl, or O—C₁₋₆ haloalkyl is not attached to the carbon in C₁₋₆ alkyl that is directly attached to the rest of the molecule,
(10) N(R^{A})-CycB,
(11) N(R^{A})-AryB,
(12) N(R^{A})—HetB,
(13) N(R^{A})—C₁₋₆ alkyl, wherein the alkyl is substituted with CycB, AryB, or HetB,
(14) C₂₋₆ alkenyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(15) C₂₋₆ alkenyl substituted with CycB, AryB, or HetB,
(16) C₂₋₆ alkynyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B}, or
(17) C₂₋₆ alkynyl substituted with CycB, AryB, or HetB;
R³ is:
(1) H,
(2) halogen,
(3) C₁₋₆ alkyl,
(4) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(5) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(6) CycC,
(7) AryC,
(8) HetC,
(9) C₁₋₆ alkyl substituted with CycC, AryC, or HetC, or
(10) C₁₋₆ alkyl substituted with Y²-CycC, Y²-AryC, or Y²-HetC;
R⁴ is:
(1) H,
(2) C₁₋₆ alkyl,
(3) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(4) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(5) CycD,
(6) AryD,
(7) HetD,
(8) C₁₋₆ alkyl substituted with CycD, AryD, or HetD, or
(9) C₁₋₆ alkyl substituted with Y³-CycD, Y³-AryD, or Y³-HetD;
R⁵ is:
(1) C₁₋₆ alkyl,
(2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(4) CycE,
(5) AryE,
(6) HetE,
(7) C₁₋₆ alkyl substituted with CycE, AryE, or HetE, or
(8) C₁₋₆ alkyl substituted with Y⁴-CycE, Y⁴-AryE, or Y⁴-HetE;
alternatively R⁴ and R⁵ together with the nitrogen atom to which they are both attached form:
(i) a 4- to 7-membered, saturated or unsaturated monocyclic ring optionally containing 1 or 2 heteroatoms in addition to the nitrogen attached to R⁴ and R⁵ selected from N, O, and S, where each S is optionally oxidized to S(O) or S(O)₂, or
(ii) a 7- to 12-membered bicyclic ring system wherein each ring in (ii) is independent of, fused to, or bridged with the other ring and each ring is saturated or unsaturated, and wherein the bicyclic ring system optionally contains from 1 to 3 heteroatoms in addition to the nitrogen attached to R⁴ and R⁵ selected from N, O, and S, where each S is optionally oxidized to S(O) or S(O)₂, and
wherein the monocyclic ring or the bicyclic ring system is optionally substituted with from 1 to 3 substituents each of which is independently:
(1) C₁₋₆ alkyl,
(2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, or SO₂N(R^{A})R^{B},
(4) O—C₁₋₆ alkyl,
(5) O—C₁₋₆ haloalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)N(R^{A})R^{B},
(17) SR^{A},
(18) S(O)R^{A},
(19) S(O)₂R^{A}, or
(20) S(O)₂N(R^{A})R^{B};
each R^{A} is independently H or C₁₋₆ alkyl;
each R^{B} is independently H or C₁₋₆ alkyl;
CycA is a carbocycle which is a C₃₋₈ cycloalkyl, a C₅₋₈ cycloalkenyl, or a C₇₋₁₂ bicyclic, saturated or unsaturated, non-aromatic ring system wherein one ring is fused to or bridged with the other ring; wherein the carbocycle is optionally substituted with a total of from 1 to 6 substituents, wherein:
(i) from zero to 6 substituents are each independently:
(1) halogen,
(2) CN
(3) C₁₋₆ alkyl,
(4) OH,
(5) O—C₁₋₆ alkyl,
(6) C₁₋₆ haloalkyl, or
(7) O—C₁₋₆ haloalkyl, and
(ii) from zero to 2 substituents are each independently:
(1) CycQ,
(2) AryQ,
(3) HetQ,
(4) HetR,
(5) Z-CycQ,
(6) Z-AryQ,
(7) Z-HetQ,
(8) Z-HetR, or
(9) C₁₋₆ alkyl substituted with CycQ, AryQ, HetQ, HetR, Z-CycQ, Z-AryQ, Z-HetQ, or Z-HetR;
AryA is aryl which is optionally substituted with a total of from 1 to 8 substituents, wherein:
(i) from zero to 8 substituents are each independently:
(1) C₁₋₆ alkyl,
(2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(4) O—C₁₋₆ alkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) S(O)₂R^{A},
(19) S(O)₂N(R^{A})R^{B},
(20) N(R^{A})S(O)₂R^{B},
(21) N(R^{A})S(O)₂N(R^{A})R^{B},
(22) N(R^{A})C(O)R^{B},
(23) N(R^{A})C(O)N(R^{A})R^{B},
(24) N(R^{A})C(O)—C(O)N(R^{A})R^{B},
(25) N(R^{A})CO₂R^{B},
(26) C₂₋₆ alkenyl, or
(27) C₂₋₆ alkynyl, and
(ii) from zero to 2 substituents are each independently:
(1) CycQ,
(2) AryQ,
(3) HetQ,
(4) HetR,
(5) Z-CycQ,
(6) Z-AryQ,
(7) Z-HetQ,
(8) Z-HetR, or
(9) C₁₋₆ alkyl substituted with CycQ, AryQ, HetQ, HetR, Z-CycQ, Z-AryQ, Z-HetQ, or Z-HetR;
HetA is a heterocycle which is optionally substituted with a total of from 1 to 8 substituents, wherein:
(i) from zero to 8 substituents are each independently:
(1)C₁₋₆ alkyl,
(2) C₁₋₆ haloalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(3) C₁₋₆ alkyl substituted with from 1 to 3 substituents each of which is OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(4) O—C₁₋₆ alkyl,
(5) O—C₁₋₆ haloalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)N(R^{A})R^{B},
(17) SR^{A},
(18) S(O)R^{A},
(19) S(O)₂R^{A},
(20) S(O)₂N(R^{A})R^{B},
(21) N(R^{A})S(O)₂R^{B},
(22) N(R^{A})S(O)₂N(R^{A})R^{B},
(23) N(R^{A})C(O)R^{B},
(24) N(R^{A})C(O)N(R^{A})R^{B},
(25) N(R^{A})C(O)—C(O)N(R^{A})R^{B}, or
(26) N(R^{A})CO₂R^{B}, and
(ii) from zero to 2 substituents are each independently:
(1) CycQ,
(2) AryQ,
(3) HetQ,
(4) HetR,
(5) Z-CycQ,
(6) Z-AryQ,
(7) HetQ,
(8) Z-HetR, or
(9) C₁₋₆ alkyl substituted with CycQ, AryQ, HetQ, HetR, Z-CycQ, Z-AryQ, Z-HetQ, or Z-HetR;
CycB, CycC, CycD and CycE each independently have the same definition as CycA;
AryB, AryC, AryD and AryE each independently have the same definition as AryA;
HetB, HetC, HetD and HetE each independently have the same definition as HetA;
each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
each heterocycle is independently (i) a 4- to 8-membered, saturated or unsaturated monocyclic ring, (ii) a 7- to 12-membered bicyclic ring system, or (iii) a 10- to 18-membered tricyclic ring system, wherein each ring in (ii) or (iii) is independent of, fused to, or bridged with the other ring or rings and each ring is saturated or unsaturated, and the monocyclic ring, bicyclic ring system, or tricyclic ring system contains from 1 to 8 heteroatoms selected from N, O and S and a balance of carbon atoms; and wherein any one or more of the nitrogen and sulfur heteroatoms is optionally oxidized, and any one or more of the nitrogen heteroatoms is optionally quaternized;
Y¹, Y², Y³ and Y⁴ are each independently selected from the group consisting of:
(i) O,
(ii) S,
(iii) S(O),
(iv) S(O)₂,
(v) O—C₁₋₆ alkylene,
(vi) S—C₁₋₆ alkylene,
(vii) S(O)—C₁₋₆ alkylene,
(viii) S(O)₂—C₁₋₆ alkylene,
(ix) N(R^{A}),
(x) N(R^{A})—C₁₋₆ alkylene,
(xi) C(O),
(xii) C(O)—C₁₋₆ alkylene,
(xiii) C(O)—C₁₋₆ alkylene-O,
(xiv) C(O)N(R^{A}),
(xv) C(O)N(R^{A})—C₁₋₆ alkylene,
(xvi) C(O)N(R^{A})—C₁₋₆ alkylene-C(O)O, and
(xvii) C(O)N(R^{A})S(O)₂;
each CycQ is independently C₃₋₈ cycloalkyl or C₅₋₈ cycloalkenyl, wherein the cycloalkyl or cycloalkenyl is optionally substituted with from 1 to 4 substituents, each of which is independently halogen, C₁₋₆ alkyl, OH, O—C₁₋₆ alkyl, C₁₋₆ haloalkyl, or O—C₁₋₆ haloalkyl;
each AryQ is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B};
each HetQ is independently (i) a 5- or 6-membered heteroaromatic ring containing from 1 to 4 heteroatoms independently selected from N, O and S, wherein each N is optionally in the form of an oxide, or (ii) a 9- or 10-membered heterobicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂; and wherein the heteroaromatic ring or the heterobicyclic ring is optionally substituted with from 1 to 4 substituents each of which is independently halogen, C₁₋₆ alkyl, C₁₋₆ haloalkyl, OH, O—C₁₋₆ alkyl, O—C₁₋₆ haloalkyl, N(R^{A})R^{B}, C(O)N(R^{A})R^{B}, C(O)R^{A}, CO₂R^{A}, SO₂R^{A}, N(R^{A})C(O)N(R^{A})R^{B}, or N(R^{A})CO₂R^{B};
each HetR is independently a 4- to 7-membered, saturated or unsaturated, non-aromatic heterocyclic ring containing at least one carbon atom and from 1 to 4 heteroatoms independently selected from N, O and S, where each S is optionally oxidized to S(O) or S(O)₂, and wherein the saturated or unsaturated heterocyclic ring is optionally substituted with from 1 to 4 substituents each of which is independently halogen, CN, C₁₋₆ alkyl, OH, oxo, O—C₁₋₆ alkyl, C₁₋₆ haloalkyl, O—C₁₋₆ haloalkyl, C(O)N(R^{A})R^{B}, C(O)R^{A}, CO₂R^{A}, or SO₂R^{A}; and
each Z is independently:
(i) O,
(ii) S,
(iii) S(O),
(iv) S(O)₂,
(v) O—C₁₋₆ alkylene,
(vi) S—C₁₋₆ alkylene,
(vii) S(O)—C₁₋₆ alkylene,
(viii) S(O)₂—C₁₋₆ alkylene,
(ix) N(R^{A}), or
(x) N(R^{A})—C₁₋₆ alkylene.

2. A compound according to claim 1, or a pharmaceutically acceptable salt thereof, wherein:
X is S, S(O), or S(O)₂;
one of R^{K} and R^{L} is H, and the other of R^{K} and R^{L} is:
(1) H,
(2) C₁₋₆ alkyl,
(3) C₁₋₆ fluoroalkyl, which is optionally substituted with O—C₁₋₆ alkyl, C(O)R^{A}, CO₂R^{A}, C(O)N(R^{A})R^{B}, SR^{A}, S(O)R^{A}, or SO₂R^{A},
(4) C₁₋₆ alkyl substituted with 1 or 2 substituents each of which is independently OH, O—C₁₋₆ alkyl, O—C₁₋₆ fluoroalkyl, CN, C(O)N(R^{A})R^{B}, C(O)R^{A}, CO₂R^{A}, SR^{A}, S(O)R^{A}, SO₂R^{A}, or SO₂N(R^{A})R^{B},
(5) CycA,
(6) AryA,
(7) HetA, or
(8) C₁₋₆ alkyl substituted with CycA, AryA, or HetA;
CycA is C₃₋₆ cycloalkyl which is optionally substituted with a total of from 1 to 3 substituents each of which is independently fluorine, C₁₋₆ alkyl, OH, O—C₁₋₆ alkyl, C₁₋₆ fluoroalkyl, or O—C₁₋₆ fluoroalkyl;
AryA is phenyl or naphthyl, wherein the phenyl or naphthyl is optionally substituted with a total of from 1 to 6 substituents wherein:
(i) from zero to 6 substituents are each independently:
(1) C₁₋₆ alkyl,
(2) C₁₋₆ fluoroalkyl,
(3) C₁₋₆ alkyl substituted with OH, O—C₁₋₄ alkyl, O—C₁₋₄ haloalkyl, CN, N(R^{A})R^{B}, C(O)N(R^{A})R^{B}, C(O)R^{A}, CO₂R^{A}, SR^{A}, S(O)R^{A}, SO₂R^{A}, or SO₂N(R^{A})R^{B},
(4) O—C₁₋₆ alkyl,
(5) O—C₁₋₆ fluoroalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₄ fluoroalkyl,
(14) CO₂R^{A},
(15) SR^{A},
(16) S(O)R^{A},
(17) SO₂R^{A}, or
(18) SO₂N(R^{A})R^{B}, and
(ii) from zero to 1 substituent is independently:
(1) CycQ,
(2) AryQ,
(3) HetQ, or
(4) C₁₋₆ alkyl substituted with CycQ, AryQ, or HetQ;
HetA is a heteroaryl which is (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂, wherein the heteroaryl is optionally substituted with a total of from 1 to 6 substituents, wherein:
(i) from zero to 6 substituents are each independently:
(1) C₁₋₆ alkyl,
(2) C₁₋₆ fluoroalkyl,
(3) C₁₋₆ alkyl substituted with OH, O—C₁₋₄ alkyl, O—C₁₋₄ haloalkyl, CN, N(R^{A})R^{B}, C(O)N(R^{A})R^{B}, C(O)R^{A}, CO₂R^{A}, SR^{A}, S(O)R^{A}, SO₂R^{A}, or SO₂N(R^{A})R^{B},
(4) O—C₁₋₆ alkyl,
(5) O—C₁₋₆ fluoroalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₄ fluoroalkyl,
(15) CO₂R^{A},
(16) SR^{A},
(17) S(O)R^{A},
(18) SO₂R^{A}, or
(19) SO₂N(R^{A})R^{B}, and
(ii) from zero to 1 substituent is independently:
(1) CycQ,
(2) AryQ,
(3) HetQ, or
(4) C₁₋₆ alkyl substituted with CycQ, AryQ, or HetQ;
R² is AryB, HetB, N(R^{A})R^{B}, or N(R^{A})-CycB;
CycB independently has the same definition as CycA;
AryB independently has the same definition as AryA;
HetB is a 4- to 7-membered saturated heterocyclic ring optionally containing from 1 to 3 heteroatoms selected from 1 to 3 N atoms, zero or 10 atom, and zero or 1 S atom, wherein the ring is attached to the rest of the compound via a N atom and the optional S atom is optionally oxidized to S(O) or S(O)₂, and wherein the saturated heterocyclic ring is optionally substituted with 1 to 3 substituents each of which is independently C₁₋₆ alkyl, oxo, C(O)N(R^{A})R^{B}, C(O)R^{A}, CO₂R^{A} or S(O)₂R^{A};
R³ is C₁₋₆ alkyl or CycC;
CycC independently has the same definition as CycA;
R⁴ is H, C₁₋₆ alkyl, or C₁₋₆ alkyl substituted with CycD, AryD, or HetD;
CycD independently has the same definition as CycA;
AryD independently has the same definition as AryA;
HetD is independently an optionally substituted heteroaryl as defined in HetA or is a 4- to 7-membered, saturated heterocyclic ring containing 1 or 2 heteroatoms selected from N, O, and S, where each S is optionally oxidized to S(O) or S(O)₂, wherein the saturated ring is optionally substituted with 1 to 3 substituents each of which is independently C₁₋₆ alkyl, OH, oxo, O—C₁₋₆ alkyl, C₁₋₆ fluoroalkyl, O—C₁₋₆ fluoroalkyl, C(O)R^{A}, CO₂R^{A}, or SO₂R^{A};
R⁵ is C₁₋₆ alkyl substituted with AryE, O-AryE, or HetE;
CycE independently has the same definition as CycA;
AryE independently has the same definition as AryA;
HetE independently has the same definition as HetD;
alternatively R⁴ and R⁵ together with the nitrogen atom to which they are both attached form a 4- to 7-membered, saturated ring optionally containing 1 heteroatom in addition to the nitrogen attached to R⁴ and R⁵ selected from N, O, and S, where the optional S is optionally oxidized to S(O) or S(O)₂; wherein the saturated ring is optionally fused to a benzene ring or a 5- or 6-membered heteroaromatic ring containing a heteroatom selected from N, O and S; and wherein the optionally fused saturated ring is optionally substituted with 1 to 3 substituents each of which is independently C₁₋₆ alkyl, OH, oxo, O—C₁₋₆ alkyl, C₁₋₆ fluoroalkyl, O—C₁₋₆ fluoroalkyl, C(O)N(R^{A})R^{B}, C(O)R^{A}, CO₂R^{A}, or SO₂R^{A};
each CycQ is independently C₃₋₆ cycloalkyl which is optionally substituted with 1 or 2 substituents, each of which is independently fluorine, C₁₋₆ alkyl, OH, O—C₁₋₆ alkyl, C₁₋₆ fluoroalkyl, or O—C₁₋₆ fluoroalkyl;
each AryQ is independently phenyl which is optionally substituted with from 1 to 3 substituents each of which is independently halogen, CN, NO₂, C₁₋₆ alkyl, C₁₋₆ fluoroalkyl, OH, O—C₁₋₆ alkyl, O—C₁₋₆ fluoroalkyl, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B}; and
each HetQ is independently a 5- or 6-membered heteroaromatic ring containing from 1 to 4 heteroatoms independently selected from N, O and S, wherein each N is optionally in the form of an oxide, wherein the heteroaromatic ring is optionally substituted with a total of from 1 to 4 substituents each of which is independently halogen, C₁₋₆ alkyl, C₁₋₆ fluoroalkyl, OH, O—C₁₋₆ alkyl, O—C₁₋₆ fluoroalkyl, N(R^{A})R^{B}, (C)ON(R^{A})R^{B}, C(O)R^{A}, CO₂R^{A}, SO₂R^{A}, N(R^{A})C(O)N(R^{A})R^{B}, or N(R^{A})CO₂R^{B}.

3. A compound according to claim 2, or a pharmaceutically acceptable salt thereof, wherein:
X is S(O)₂;
one of R^{K} and R^{L} is H, and the other is:
(1) H,
(2) C₁₋₄ alkyl,
(3) C₁₋₄ fluoroalkyl, which is optionally substituted with O—C₁₋₄ alkyl or CO₂R^{A},
(4) C₁₋₄ alkyl substituted with O—C₁₋₄ alkyl, O—C₁₋₄ fluoroalkyl, C(O)R^{A}, CO₂R^{A}, Or SO₂R^{A}, or
(5) C₁₋₄ alkyl substituted with CycA, AryA, or HetA;
CycA is C₃₋₆ cycloalkyl which is optionally substituted with 1 or 2 substituents each of which is independently C₁₋₄ alkyl, OH, O—C₁₋₄ alkyl, C₁₋₄ fluoroalkyl, or O—C₁₋₄ fluoroalkyl;
AryA is phenyl which is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A};
HetA is a heteroaryl selected from the group consisting of thienyl, furanyl, pyrrolyl, imidazolyl, pyrazolyl, triazolyl, tetrazolyl, oxazolyl, isooxazolyl, thiazolyl, isothiazolyl, oxadiazolyl, thiadiazolyl, pyridinyl, pyrazinyl, pyrimidinyl, pyridazinyl, benzofuranyl, benzothienyl, indolyl, indazolyl, isobenzofuranyl, benzisoxazolyl, benzoxazolyl, benzimidazolyl, benzopiperidinyl, chromenyl, quinolinyl, isoquinolinyl, cinnolinyl, quinoxalinyl, quinazolinyl, and imidazopyridinyl, wherein the heteroaryl is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A};
R² is AryB, HetB, or N(R^{A})-CycB;
CycB is C₃₋₆ cycloalkyl which is optionally substituted with 1 or 2 substituents each of which is independently C₁₋₄ alkyl, OH, O—C₁₋₄ alkyl, C₁₋₄ fluoroalkyl, or O—C₁₋₄ fluoroalkyl;
AryB is phenyl or naphthyl, wherein the phenyl is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A};
HetB is a saturated heterocyclic ring selected from the group consisting of azetidinyl, pyrrolidinyl, piperidinyl, morpholinyl, thiomorpholinyl, piperazinyl, thiazinanyl, thiazepanyl and azepanyl, wherein the ring is attached to the rest of the compound via a ring nitrogen atom, and wherein the ring is optionally substituted with from 1 to 3 substituents each of which is independently C₁₋₄ alkyl or oxo;
R³ is C₁₋₄ alkyl;
R⁴ is H, C₁₋₄ alkyl, or C₁₋₄ alkyl substituted with AryD;
AryD is phenyl which is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A};
R⁵ is C₁₋₄ alkyl substituted with AryE, O-AryE, or HetE;
AryE is phenyl which is optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A};
HetE is independently:
(i) a heteroaryl selected from the group consisting of thienyl, furanyl, pyrrolyl, imidazolyl, pyrazolyl, triazolyl, tetrazolyl, oxazolyl, isooxazolyl, thiazolyl, isothiazolyl, oxadiazolyl, thiadiazolyl, pyridinyl, pyrazinyl, pyrimidinyl, pyridazinyl, benzofuranyl, benzothienyl, indolyl, indazolyl, isobenzofuranyl, benzisoxazolyl, benzoxazolyl, benzimidazolyl, benzopiperidinyl, chromenyl, quinolinyl, isoquinolinyl, cinnolinyl, quinoxalinyl, quinazolinyl, and imidazopyridinyl, wherein the heteroaryl is (a) optionally substituted with from 1 to 3 substituents each of which is independently C₁₋₄ alkyl, CF₃, O—C₁₋₄ alkyl, OCF₃, OH, halogen, CN, NO₂, N(R^{A})R^{B}, C(O)N(R^{A})R^{B}, C(O)R^{A}, C(O)CF₃, CO₂R^{A}, or SO₂R^{A}, and (b) additionally and optionally substituted with phenyl, or
(ii) a saturated heterocyclic ring selected from the group consisting of azetidinyl, pyrrolidinyl, piperidinyl, morpholinyl, thiomorpholinyl, piperazinyl, thiazinanyl, thiazepanyl and azepanyl, wherein the ring is attached to the rest of the compound via a ring carbon atom, and wherein the ring is optionally substituted with 1 to 3 substituents each of which is independently C₁₋₄ alkyl or oxo;
alternatively R⁴ and R⁵ together with the nitrogen atom to which they are both attached form a heterocyclic ring optionally having a benzo or thieno ring fused thereto, which is selected from the group consisting of 1-azetidinyl 1-pyrrolidinyl, 1-piperidinyl, 1-piperazinyl, 1-azepanyl, 4-morpholinyl, 4-thiomorpholinyl, 3,4-dihydroisoquinolin-2(1H)-yl, 1,3,4,5-tetrahydro-2H-2-benzazepin-2-yl and 4,6,7,8-tetrahydro-5H-thieno[3,2-c}azepin-5-yl; wherein the optionally fused heterocyclic ring is optionally substituted with 1 or 2 substituents each of which is independently C₁₋₄ alkyl, OH, oxo, halogen, O—C₁₋₄ alkyl, or SO₂—C₁₋₄ alkyl; and
R^{A} and R^{B} are each independently H or C₁₋₄ alkyl.

4. A compound according to claim 3, or a pharmaceutically acceptable salt thereof, wherein:
R^{K} is H;
R^{L} is H, C₁₋₄ alkyl, CH₂CF₃, CH₂CH₂CF₃, CH₂CF₂CF₃, CH(CO₂CH₃)CH₂CF₃, (CH₂)₂₋₃OCH₃, CH₂-AryA, or CH₂-HetA;
AryA is phenyl which is optionally substituted with from 1 to 3 substituents each of which is independently CH₃, CF₃, OCH₃, OCF₃, OH, Cl, Br, F, CN, NO₂, NH₂, N(H)CH₃, N(CH₃)₂, C(O)NH₂, C(O)N(H)CH₃, C(O)N(CH₃)₂, C(O)CH₃, C(O)CF₃, CO₂CH₃, or SO₂CH₃;
HetA is a heteroaryl selected from the group consisting of pyridinyl, pyrrolyl, thienyl, furanyl, imidazolyl, pyrazolyl, triazolyl, tetrazolyl, oxazolyl, isooxazolyl, oxadiazolyl, thiazolyl, isothiazolyl, thiadiazolyl, benzofuranyl, benzothienyl, indolyl, indazolyl, isobenzofuranyl, benzoxazolyl, benzimidazolyl, quinolinyl, isoquinolinyl, wherein the heteroaryl is optionally substituted with from 1 to 3 substituents each of which is independently CH₃, CF₃, OCH₃, OCF₃, OH, Cl, Br, F, CN, C(O)NH₂, C(O)N(H)CH₃, C(O)N(CH₃)₂, C(O)CH₃, C(O)CF₃, CO₂CH₃, or SO₂CH₃;
R² is AryB or HetB;
CycB is C₃₋₆ cycloalkyl;
AryB is phenyl or naphthyl, wherein the phenyl is optionally substituted with from 1 to 3 substituents each of which is independently CH₃, CF₃, OCH₃, OCF₃, OH, Cl, Br, F, CN, NO₂, NH₂, N(H)CH₃, N(CH₃)₂, C(O)NH₂, C(O)N(H)CH₃, C(O)N(CH₃)₂, C(O)CH₃, C(O)CF₃, CO₂CH₃, or SO₂CH₃;
HetB is a saturated heterocyclic ring selected from the group consisting of: 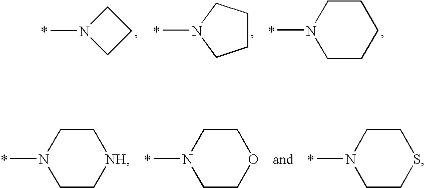
wherein the asterisk * denotes the point of attachment to the rest of the compound, and wherein the ring is optionally substituted with 1 or 2 substituents each of which is CH₃ or oxo;
R³ is CH₃, CH₂CH₃, CH₂CH₂CH₃, CH(CH₃)₂, or CH₂CH₂CH₂CH₃;
R⁴ is H, CH₃, CH₂CH₃, or benzyl;
R⁵ is CH₂-AryE, CH₂CH₂-AryE, CH(CH₃)-AryE, CH₂O-AryE, CH₂CH₂O-AryE, CH₂-HetE, or CH₂CH₂-HetE;
AryE is phenyl which is optionally substituted with from 1 to 3 substituents each of which is independently CH₃, CF₃, OCH₃, OCF₃, OH, Cl, Br, F, CN, NO₂, NH₂, N(H)CH₃, N(CH₃)₂, C(O)NH₂, C(O)N(H)CH₃, C(O)N(CH₃)₂, C(O)CH₃, C(O)CF₃, CO₂CH₃, or SO₂CH₃;
HetE is independently:
(i) a heteroaryl selected from the group consisting of thienyl, furanyl, pyrrolyl, imidazolyl, pyrazolyl, triazolyl, tetrazolyl, oxazolyl, isooxazolyl, thiazolyl, isothiazolyl, oxadiazolyl, thiadiazolyl, pyridinyl, pyrazinyl, pyrimidinyl, pyridazinyl, benzofuranyl, benzothienyl, indolyl, indazolyl, isobenzofuranyl, benzisoxazolyl, benzoxazolyl, benzimidazolyl, benzopiperidinyl, chromenyl, quinolinyl, isoquinolinyl, cinnolinyl, quinoxalinyl, quinazolinyl, and imidazopyridinyl, wherein the heteroaryl is (a) optionally substituted with from 1 to 3 substituents each of which is independently CH₃, CF₃, OCH₃, OCF₃, OH, Cl, Br, F, CN, NO₂, NH₂, N(H)CH₃, N(CH₃)₂, C(O)NH₂, C(O)N(H)CH₃, C(O)N(CH₃)₂, C(O)CH₃, C(O)CF₃, CO₂CH₃, or SO₂CH₃, and (b) additionally and optionally substituted with phenyl, or
(ii) a saturated heterocyclic ring selected from the group consisting of azetidinyl, pyrrolidinyl, piperidinyl, morpholinyl, thiomorpholinyl, piperazinyl, thiazinanyl, thiazepanyl and azepanyl, wherein the ring is attached to the rest of the compound via a ring carbon atom, and wherein the ring is optionally substituted with 1 or 2 substituents each of which is CH₃ or oxo;
alternatively R⁴ and R⁵ together with the nitrogen atom to which they are both attached form a heterocyclic ring optionally having a benzo or thieno ring fused thereto, which is selected from the group consisting of 1-azetidinyl, 1-pyrrolidinyl, 1-piperidinyl, 1-piperazinyl, 1-azepanyl, 4-morpholinyl, 4-thiomorpholinyl, 3,4-dihydroisoquinolin-2(1H)-yl, 1,3,4,5-tetrahydro-2H-2-benzazepin-2-yl and 4,6,7,8-tetrahydro-5H-thieno[3,2-c]azepin-5-yl; wherein the optionally fused heterocyclic ring is optionally substituted with 1 or 2 substituents each of which is independently CH₃, OH, oxo, Cl, Br. F, OCH₃, or SO₂CH₃.

5. A compound according to claim 1 selected from the group consisting of:
N-(2,4-dichlorobenzyl)-N,3-dimethyl-4-(1-phenylsulfonyl)-1H-pyrrole-2,5-dicarboxamide;
N-(2,4-dichlorobenzyl)-4-[(3,5-dichlorophenyl)sulfonyl]-N,3-dimethyl-1H-pyrrole-2,5-dicarboxamide;
N-benzyl-N,3-dimethyl-4-(3,5-dimethylphenylsulfonyl)-1H-pyrrole-2,5-dicarboxamide;
N-(2-chloro-4-fluorobenzyl)-N,3-dimethyl-4-(phenylsulfonyl)-1H-pyrrole-2,5-dicarboxamide;
N-(2-chlorobenzyl)-N,3-dimethyl-4-(1-naphthylsulfonyl)-1H-pyrrole-2,5-dicarboxamide;
N-(2-chlorobenzyl)-N,3-dimethyl-4-(2-naphthylsulfonyl)-1H-pyrrole-2,5-dicarboxamide;
N-(2-fluorobenzyl)-N,3-dimethyl-4-(phenylsulfonyl)-1H-pyrrole-2,5-dicarboxamide;
N-(2-chlorobenzyl)-N,3-dimethyl-4-(phenylsulfonyl)-1H-pyrrole-2,5-dicarboxamide;
N-(2-bromobenzyl)-N,3-dimethyl-4-(phenylsulfonyl)-1H-pyrrole-2,5-dicarboxamide;
N-(3-chlorobenzyl)-N,3-dimethyl-4-(phenylsulfonyl)-1H-pyrrole-2,5-dicarboxamide;
N-(4-bromobenzyl)-N,3-dimethyl-4-(phenylsulfonyl)-1H-pyrrole-2,5-dicarboxamide;
N-(4-chloro-2-fluorobenzyl)-N,3-dimethyl-4-(phenylsulfonyl)-1H-pyrrole-2,5-dicarboxamide;
N-(2,3-dichlorobenzyl)-N,3-dimethyl-4-(phenylsulfonyl)-1H-pyrrole-2,5-dicarboxamide;
N-(3,4-dichlorobenzyl)-N,3-dimethyl-4-(phenylsulfonyl)-1H-pyrrole-2,5-dicarboxamide;
N-(2-chloro-4-methylsulfonylbenzyl)-N,3-dimethyl-4-(phenylsulfonyl)-1H-pyrrole-2,5-dicarboxamide;
N-(2-fluorobenzyl)-N,3-dimethyl-4-(3-fluorophenylsulfonyl)-1H-pyrrole-2,5-dicarboxamide;
N-(2-chlorobenzyl)-N,3-dimethyl-4-(3-fluorophenylsulfonyl)-1H-pyrrole-2,5-dicarboxamide;
N-(4-chloro-2-fluorobenzyl)-N,3-dimethyl-4-(3-fluorophenylsulfonyl)-1H-pyrrole-2,5-dicarboxamide;
N-(2,4-dichlorobenzyl)-N,3-dimethyl-4-(3-fluorophenylsulfonyl)-1H-pyrrole-2,5-dicarboxamide;
N-(2-chlorobenzyl)-N,3-dimethyl-4-(3-chlorophenylsulfonyl)-1H-pyrrole-2,5-dicarboxamide;
N-(2,4-dichlorobenzyl)-N,3-dimethyl-4-(3-chlorophenylsulfonyl)-1H-pyrrole-2,5-dicarboxamide;
N-benzyl-N,3-dimethyl-4-(3-trifluoromethylphenylsulfonyl)-1H-pyrrole-2,5-dicarboxamide;
N-(2,4-dichlorobenzyl)-N,3-dimethyl-4-(3-trifluoromethylphenylsulfonyl)-1H-pyrrole-2,5-dicarboxamide;
N-(2-fluorobenzyl)-N,3-dimethyl-4-(3,5-dimethylphenylsulfonyl)-1H-pyrrole-2,5-dicarboxamide;
N-(2-chlorobenzyl)-N,3-dimethyl-4-(3,5-dimethylphenylsulfonyl)-1H-pyrrole-2,5-dicarboxamide;
N-(4-chloro-2-fluorobenzyl)-N,3-dimethyl-4-(3,5-dimethylphenylsulfonyl)-1H-pyrrole-2,5-dicarboxamide;
N-(2,4-dichlorobenzyl)-N,3-dimethyl-4-(3,5-dimethylphenylsulfonyl)-1H-pyrrole-2,5-dicarboxamide;
N-benzyl-N,3-dimethyl-4-(3-chloro-5-fluorophenylsulfonyl)-1H-pyrrole-2,5-dicarboxamide;
N-(2-fluorobenzyl)-N,3-dimethyl-4-(3-chloro-5-fluorophenylsulfonyl)-1H-pyrrole-2,5-dicarboxamide;
N-(2-chlorobenzyl)-N,3-dimethyl-4-(3-chloro-5-fluorophenylsulfonyl)-1H-pyrrole-2,5-dicarboxamide;
N-(4-chloro-2-fluorobenzyl)-N,3-dimethyl-4-(3-chloro-5-fluorophenylsulfonyl)-1H-pyrrole-2,5-dicarboxamide;
N-benzyl-N,3-dimethyl-4-(3,5-dichlorophenylsulfonyl)-1H-pyrrole-2,5-dicarboxamide;
N-(2-fluorobenzyl)-N,3-dimethyl-4-(3,5-dichlorophenylsulfonyl)-1H-pyrrole-2,5-dicarboxamide;
N-(2-chlorobenzyl)-N,3-dimethyl-4-(3,5-dichlorophenylsulfonyl)-1H-pyrrole-2,5-dicarboxamide;
N-(4-chloro-2-fluorobenzyl)-N,3-dimethyl-4-(3,5-dichlorophenylsulfonyl)-1H-pyrrole-2,5-dicarboxamide;
N-(3-methoxyobenzyl)-N,3-dimethyl-4-(phenylsulfonyl)-1H-pyrrole-2,5-dicarboxamide;
N-(2-chlorobenzyl)-N,3-dimethyl-4-(2-cyano-3-methylphenylsulfonyl)-1H-pyrrole-2,5-dicarboxamide;
N,3-dimethyl-4-(phenylsulfonyl)-N-(3-thienylmethyl)-1H-pyrrole-2,5-dicarboxamide;
N-[(3-chloro-4-pyridinyl)methyl]-N,3-dimethyl-4-(phenylsulfonyl)-1H-pyrrole-2,5-dicarboxamide; (trifluoracetic acid salt)
4-[(3,5-dimethylphenyl)sulfonyl]-N,3-dimethyl-N-(6-quinolinylmethyl)-1H-pyrrole-2,5-dicarboxamide;
N-[(7-chloro-6-quinolinyl)methyl]-N,3-dimethyl-4-(phenylsulfonyl)-1H-pyrrole-2,5-dicarboxamide;
N-[(5-chloro-6-quinolinyl)methyl]-N,3-dimethyl-4-(phenylsulfonyl)-1H-pyrrole-2,5-dicarboxamide;
N,3-dimethyl-4-(phenylsulfonyl-N-(6-quinolinylmethyl)-1H-pyrrole-2,5-dicarboxamide;
N,3-dimethyl-4-(3-methylphenylsulfonyl-N-(6-quinolinylmethyl)-1H-pyrrole-2,5-dicarboxamide;
4-(3-fluorophenylsulfonyl-N,3-dimethyl-N-(6-quinolinylmethyl)-1H-pyrrole-2,5-dicarboxamide;
N,3-dimethyl-N-(6-quinolinylmethyl)-4-{[3-(trifluoromethyl)phenyl]sulfonyl}-1H-pyrrole-2,5-dicarboxamide;
N-[(5-chloro-6-quinolinyl)methyl]-4-[(3,5-dimethylphenyl)sulfonyl]-N,3-dimethyl-1H-pyrrole-2,5-dicarboxamide;
N-[(7-chloro-6-quinolinyl)methyl]-4-[(3,5-dimethylphenyl)sulfonyl]-N,3-dimethyl-1H-pyrrole-2,5-dicarboxamide;
N-[(5-chloro-6-quinolinyl)methyl]-4-[(3,5-difluorophenyl)sulfonyl]-N,3-dimethyl-1H-pyrrole-2,5-dicarboxamide;
N-[(7-chloro-6-quinolinyl)methyl]-4-[(3,5-difluorophenyl)sulfonyl]-N,3-dimethyl-1H-pyrrole-2,5-dicarboxamide;
4-[(3-chloro-5-fluorophenyl)sulfonyl]-N,3-dimethyl-N-(6-quinolinylmethyl)-1H-pyrrole-2,5-dicarboxamide;
4-[(3-chloro-5-fluorophenyl)sulfonyl]-N-[(5-chloro-6-quinolinyl)methyl]-N,3-dimethyl-1H-pyrrole-2,5-dicarboxamide;
4-[(3-chloro-5-fluorophenyl)sulfonyl]-N-[(7-chloro-6-quinolinyl)methyl]-N,3-dimethyl-1H-pyrrole-2,5-dicarboxamide;
4-[(3,5-dichlorophenyl)sulfonyl]-N,3-dimethyl-N-(6-quinolinylmethyl)-1H-pyrrole-2,5-dicarboxamide;
N-[(5-chloro-6-quinolinyl)methyl]-4-[(3,5-dichlorophenyl)sulfonyl]-N,3-dimethyl-1H-pyrrole-2,5-dicarboxamide;
N-[(7-chloro-6-quinolinyl)methyl]-4-[(3,5-dichlorophenyl)sulfonyl]-N,3-dimethyl-1H-pyrrole-2,5-dicarboxamide;
4-[(3-chloro-5-cyanophenyl)sulfonyl]-N,3-dimethyl-N-[(6-quinolinylmethyl]-1H-pyrrole-2,5-dicarboxamide;
N-benzyl-3-isopropyl-N-methyl-4-(phenylsulfonyl)-1H-pyrrole-2,5-dicarboxamide;
N-(2,4-dichlorobenzyl)-3-ethyl-N-methyl-4-(phenylsulfonyl)-1H-pyrrole-2,5-dicarboxamide;
3-ethyl-N-methyl-4-(phenylsulfonyl)-N-(6-quinolinylmethyl)-1H-pyrrole-2,5-dicarboxamide;
N-(2-fluorobenzyl)-3-isopropyl-N-methyl-4-(phenylsulfonyl)-1H-pyrrole-2,5-dicarboxamide;
N-(4-chloro-2-fluorobenzyl)-3-isopropyl-N-methyl-4-(phenylsulfonyl)-1H-pyrrole-2,5-dicarboxamide;
N-(2,4-dichlorobenzyl)-3-isopropyl-N-methyl-4-(phenylsulfonyl)-1H-pyrrole-2,5-dicarboxamide;
3-isopropyl-N-methyl-4-(phenylsulfonyl)-N-(6-quinolinylmethyl)-1H-pyrrole-2,5-dicarboxamide;
N-methyl-4-(phenylsulfonyl)-3-propyl-N-(6-quinolinylmethyl)-1H-pyrrole-2,5-dicarboxamide;
3-butyl-N-methyl-4-(phenylsulfonyl)-N-(6-quinolinylmethyl)-1H-pyrrole-2,5-dicarboxamide;
3-butyl-N-methyl-4-(3-methylphenylsulfonyl)-N-(6-quinolinylmethyl)-1H-pyrrole-2,5-dicarboxamide;
3-butyl-4-[(3-chlorophenyl)sulfonyl]-N-methyl-N-(6-quinolinylmethyl)-1H-pyrrole-2,5-dicarboxamide;
3-butyl-4-[(3,5-dimethylphenyl)sulfonyl]-N-methyl-N-(6-quinolinylmethyl)-1H-pyrrole-2,5-dicarboxamide;
3-butyl-4-[(3,5-dichlorophenyl)sulfonyl]-N-methyl-N-(6-quinolinylmethyl)-1H-pyrrole-2,5-dicarboxamide;
N-(2,4-dichlorobenzyl)-N′-(1H-indazol-3-ylmethyl)-N,3-dimethyl-4-(phenylsulfonyl)-1H-pyrrole-2,5-dicarboxamide;
N-(2,4-dichlorobenzyl)-N,N′,3-trimethyl-4-(phenylsulfonyl)-1H-pyrrole-2,5-dicarboxamide;
N-(2,4-dichlorobenzyl)-N,3-dimethyl-N′-[(3-methyl-4-pyridinyl)methyl]-4-(phenylsulfonyl)-1H-pyrrole-2,5-dicarboxamide;
N-(2,4-dichlorobenzyl)-N,3-dimethyl-4-(phenylsulfonyl)-N′-(2-pyridinylmethyl)-1H-pyrrole-2,5-dicarboxamide;
N-(2,4-dichlorobenzyl)-N,3-dimethyl-4-(phenylsulfonyl)-N′-(3-pyridinylmethyl)-1H-pyrrole-2,5-dicarboxamide;
N-(2,4-dichlorobenzyl)-N,3-dimethyl-4-(phenylsulfonyl)-N′-(1,3-thiazol-2-ylmethyl)-1H-pyrrole-2,5-dicarboxamide;
N′-(2-chloro-6-fluorobenzyl)-N-(2,4-dichlorobenzyl)-N,3-dimethyl-4-(phenylsulfonyl)-1H-pyrrole-2,5-dicarboxamide;
4-methyl-3-(phenylsulfonyl)-5-(1,3,4,5-tetrahydro-2H-2-benzazepine-2-ylcarbonyl)-1H-pyrrole-2-carboxamide;
4-methyl-3-(3,5-dimethylphenylsulfonyl)-5-(1,3,4,5-tetrahydro-2H-2-benzazepine-2-ylcarbonyl)-1H-pyrrole-2-carboxamide;
3-[(3,5-dimethylphenyl)sulfonyl]-4-methyl-5-(4,6,7,8-tetrahydro-5H-thieno[3,2-c]azepin-5-ylcarbonyl)-1H-pyrrole-2-carboxamide;
3-[(3,5-dichlorophenyl)sulfonyl]-4-methyl-5-(1,3,4,5-tetrahydro-2H-2-benzazepin-2-ylcarbonyl)-1H-pyrrole-2-carboxamide;
3-[(3,5-dichlorophenyl)sulfonyl]-4-methyl-5-(4,6,7,8-tetrahydro-5H-thieno[3,2-c]azepin-5-ylcarbonyl)-1H-pyrrole-2-carboxamide;
N-(2,4-dichlorobenzyl)-N,3-dimethyl-4-(1-pyrrolidinylsulfonyl)-1H-pyrrole-2,5-dicarboxamide;
N-(2-chlorobenzyl)-N,3-dimethyl-N′-(2-pyridinylmethyl)-4-(1-pyrrolidinylsulfonyl)-1H-pyrrole-2,5-dicarboxamide;
N-(2,4-dichlorobenzyl)-N,3-dimethyl-4-(1-piperidinylsulfonyl)-1H-pyrrole-2,5-dicarboxamide;
N-(2-chlorobenzyl)-N,3-dimethyl-4-(1-piperidinylsulfonyl)-1H-pyrrole-2,5-dicarboxamide;
and pharmaceutically acceptable salts thereof.

6. A compound according to claim 1 selected from the group consisting of:
3-isopropyl-N-methyl-4-(phenylsulfonyl)-N-[(5-chloroquinolin-6-yl)methyl]-1H-pyrrole-2,5-dicarboxamide (trifluoroacetic acid salt);
3-isopropyl-N-methyl-4-(phenylsulfonyl)-N-[(7-chloroquinolin-6-yl)methyl]-1H-pyrrole-2,5-dicarboxamide (trifluoroacetic acid salt);
3-[(3,5-dichlorophenyl)sulfonyl]-4-methyl-5-(1,3,4,5-tetrahydro-2H-2-benzazepin-2-ylcarbonyl)-1H-pyrrole-2-carboxamide;
3-[(3,5-dichlorophenyl)sulfonyl]-4-methyl-5-(4,6,7,8-tetrahydro-5H-thieno[3,2-c]azepin-5-ylcarbonyl)-1H-pyrrole-2-carboxamide;
3-[(3,5-dichlorophenyl)sulfonyl]-4-methyl-5-(7-methoxy-1,3,4,5-tetrahydro-2H-2-benzazepin-2-ylcarbonyl)-1H-pyrrole-2-carboxamide;
3-[(3,5-dichlorophenyl)sulfonyl]-4-methyl-5-(7-hydroxy-1,3,4,5-tetrahydro-2H-2-benzazepin-2-ylcarbonyl)-1H-pyrrole-2-carboxamide;
3-[(3,5-dimethylphenyl)sulfonyl]-4-methyl-5-(7-methoxy-1,3,4,5-tetrahydro-2H-2-benzazepin-2-ylcarbonyl)-1H-pyrrole-2-carboxamide;
3-[(3,5-dimethylphenyl)sulfonyl]-4-methyl-5-(7-hydroxy-1,3,4,5-tetrahydro-2H-2-benzazepin-2-ylcarbonyl)-1H-pyrrole-2-carboxamide;
3-[(3,5-dimethylphenyl)sulfonyl]-4-methyl-5-(8-methoxy-1,3,4,5-tetrahydro-2H-2-benzazepin-2-ylcarbonyl)-1H-pyrrole-2-carboxamide;
3-[(3,5-dimethylphenyl)sulfonyl]-4-methyl-5-(8-hydroxy-1,3,4,5-tetrahydro-2H-2-benzazepin-2-ylcarbonyl)-1H-pyrrole-2-carboxamide;
3-(phenylsulfonyl)-4-methyl-5-(8-methoxy-1,3,4,5-tetrahydro-2H-2-benzazepin-2-ylcarbonyl)-1H-pyrrole-2-carboxamide;
3-(phenylsulfonyl)-4-methyl-5-(8-hydroxy-1,3,4,5-tetrahydro-2H-2-benzazepin-2-ylcarbonyl)-1H-pyrrole-2-carboxamide;
3-[(3-fluorophenyl)sulfonyl]-4-methyl-5-(8-methoxy-1,3,4,5-tetrahydro-2H-2-benzazepin-2-ylcarbonyl)-1H-pyrrole-2-carboxamide;
3-[(3-fluorophenyl)sulfonyl]-4-methyl-5-(8-hydroxy-1,3,4,5-tetrahydro-2H-2-benzazepin-2-ylcarbonyl)-1H-pyrrole-2-carboxamide;
3-[(3-(1,1,1-trifluoromethyl)phenyl)sulfonyl]-4-methyl-5-(8-methoxy-1,3,4,5-tetrahydro-2H-2-benzazepin-2-ylcarbonyl)-1H-pyrrole-2-carboxamide;
3-[(3,5-Dichlorophenyl)sulfonyl]-4-methyl-5-[(2-chloro-4,6,7,8-tetrahydro-5H-thieno[3,2-c]azepin-5-yl)carbonyl]-1H-pyrrole-2-carboxamide;
3-[(3,5-Dichlorophenyl)sulfonyl]-4-methyl-5-[(2-(methylsulfonyl)-4,6,7,8-tetrahydro-5H-thieno[3,2-c]azepin-5-yl)carbonyl]-1H-pyrrole-2-carboxamide;
3-[(3,5-Dimethylphenyl)sulfonyl]-4-methyl-5-[(2-(methylsulfonyl)-4,6,7,8-tetrahydro-5H-thieno[3,2-c]azepin-5-yl)carbonyl]-1H-pyrrole-2-carboxamide;
N5-[(2-aminopyridin-4-yl)methyl]-3-[(3-fluorophenyl)sulfonyl]-4-isopropyl-N5-methyl-1H-pyrrole-2,5-dicarboxamide;
N5-[(2-aminopyridin-4-yl)methyl]-3-[(3,5-difluorophenyl)sulfonyl]-4-isopropyl-N5-methyl-1H-pyrrole-2,5-dicarboxamide;
N5-[(2-aminopyridin-4-yl)methyl]-3-phenylsulfonyl]-4-isopropyl-N5-methyl-1H-pyrrole-2,5-dicarboxamide;
N5-[(2-aminopyridin-4-yl)methyl]-3-[(3,5-dimethylphenyl)sulfonyl]-4-isopropyl-N5-methyl-1H-pyrrole-2,5-dicarboxamide;
N5-[(2-amino-5-fluoropyridin-4-yl)methyl]-3-[(3,5-dimethylphenyl)sulfonyl]-N5,4-dimethyl-1H-pyrrole-2,5-dicarboxamide;
N2-[(3-N5-[(2-amino-5-fluoropyridin-4-yl)methyl]-3-isopropyl-N2-methyl-4-(phenylsulfonyl)-1H-pyrrole-2,5-dicarboxamide;
N5-[(3-chloropyridin-4-yl)methyl]-3-[(3,5-dimethylphenyl)sulfonyl]-N5,4-dimethyl-1H-pyrrole-2,5-dicarboxamide; (free base)
N2-[(3-chloropyridin-4-yl)methyl]-3-isopropyl-N2-methyl-4-(phenylsulfonyl)-1H-pyrrole-2,5-dicarboxamide;
N5-[(2-chloro-3-fluoropyridin-4-yl)methyl]-3-[(3,5-dimethylphenyl)sulfonyl]-N5,4-dimethyl-1H-pyrrole-2,5-dicarboxamide;
N2-[(2-chloro-3-fluoropyridin-4-yl)methyl]-3-isopropyl-N2-methyl-4-(phenylsulfonyl)-1H-pyrrole-2,5-dicarboxamide;
3-[(3,5-dimethylphenyl)sulfonyl]-N-5-[(3-fluoropyridin-4-yl)methyl]-N5,4-dimethyl-1H-pyrrole-2,5-dicarboxamide;
N2-[(3-fluoropyridin-4-yl)methyl]-3-isopropyl-N2-methyl-4-(phenylsulfonyl)-1H-pyrrole-2,5-dicarboxamide;
N5-[(3,5-difluoropyridin-4-yl)methyl]-3-[(3,5-dimethylphenyl)sulfonyl]-N5,4-dimethyl-1H-pyrrole-2,5-dicarboxamide;
N2-[(3,5-difluoropyridin-4-yl)methyl]-3-isopropyl-N2-methyl-4-(phenylsulfonyl)-1H-pyrrole-2,5-dicarboxamide;
N5-[(2-chloro-5-fluoropyridin-4-yl)methyl]-3-[(3,5-dimethylphenyl)sulfonyl]-N5,4-dimethyl-1H-pyrrole-2,5-dicarboxamide;
N2-[(2-chloro-5-fluoropyridin-4-yl)methyl]-3-isopropyl-N2-methyl-4-(phenylsulfonyl)-1H-pyrrole-2,5-dicarboxamide;
N2-[(2-amino-3-fluoropyridin-4-yl)methyl]-3-isopropyl-N2-methyl-4-(phenylsulfonyl)-1H-pyrrole-2,5-dicarboxamide;
and pharmaceutically acceptable salts thereof.

7. A pharmaceutical composition comprising an effective amount of a compound according to claim 1, or a pharmaceutically acceptable salt thereof, and a pharmaceutically acceptable carrier.

8. A pharmaceutical combination which is (i) a compound according to claim 1, or a pharmaceutically acceptable salt thereof, and (ii) an HIV infection/AIDS antiviral agent selected from the group consisting of HIV protease inhibitors, nucleoside HIV reverse transcriptase inhibitors, and non-nucleoside HIV reverse transcriptase inhibitors; wherein the compound of (i) or its pharmaceutically acceptable salt and the HIV infection/AIDS antiviral agent of (ii) are each employed in an amount that renders the combination effective for the treatment or prophylaxis of HIV infection or the treatment or prophylaxis or delay in the onset or progression of AIDS.

9. A method for the inhibition of HIV reverse transcriptase, the treatment or prophylaxis of HIV infection, or the treatment or prophylaxis or delay in the onset or progression of AIDS, wherein the method comprises administering to a subject in need thereof an effective amount of a compound of Formula I, or a pharmaceutically acceptable salt thereof, as defined in claim 1.

10. (canceled)

11. (canceled)
